(19) United States
(12) Patent Application Publication
(10) Pub. No.: US 2004/0248840 A1
(43) Pub. Date: Dec. 9, 2004

(54) Oligomeric compounds for the modulation of ras expression

(75) Inventors: Bo Hansen, Copenhagen K (DK); Charlotte Albaek Thrue, Copenhagen K (DK); Majken Westergaard, Birkered (DK); Kamille Dumong Petersen, Lejre (DK); Margit Wissenbach, Fredensborg (DK)

Correspondence Address: Peter F. Corless, EDWARDS & ANGELL, LLP, P.O. Box 55874, Boston, MA, 02205, US

(73) Assignee: Santaris Pharma A/S

(21) Appl. No.: 10/776,917

(22) Filed: Feb. 10, 2004

Related U.S. Application Data
(60) Provisional application No. 60/446,363, filed on Feb. 10, 2003.

(30) Foreign Application Priority Data
Oct. 20, 2003 (DK) PA 2003 01539

(51) Int. Cl. A61K 48/00; C07H 21/04

(52) U.S. Cl. 514/44; 536/23.1; 544/244

(57) ABSTRACT

Oligonucleotides directed against the Ha-ras gene are provided for modulating the expression of Ha-ras. The compositions comprise oligonucleotides, particularly antisense oligonucleotides, targeted to nucleic acids encoding the Ha-ras. Methods of using these compounds for modulation of Ha-ras expression and for the treatment of diseases associated with either overexpression of Ha-ras, expression of mutated Ha-ras or both are provided. Examples of diseases are cancer such as lung, breast, colon, prostate, pancreas, lung, liver, thyroid, kidney, brain, testes, stomach, intestine, bowel, spinal cord, sinuses, bladder, urinary tract or ovaries cancers. The oligonucleotides may be composed of deoxyribonucleosides or a nucleic acid analogue such as for example locked nucleic acid or a combination thereof.

### FIELD OF THE INVENTION

The present invention provides compositions and methods for modulating the expression of the ras family of proto-oncogenes, preferably Ha-ras, Ki-ras and N-ras, most preferably Ha-ras. In particular, this invention relates to oligomeric compounds and preferred such compounds are oligonucleotides, which are specifically hybridisable with nucleic acids encoding ras. The oligonucleotide compounds have been shown to modulate the expression of ras and pharmaceutical preparations thereof and their use as treatment of cancer diseases are disclosed.

### BACKGROUND OF THE INVENTION

The ras proto-oncogenes encode a group of plasma membrane-associated G-proteins that bind guanine nucleotides with high affinity and activates several downstream effector proteins including raf-1, PI3-K etc. that are known to activate several distinct signalling cascades that are involved in the regulation of cellular survival, proliferation and differentiation in response to extracellular stimuli such as growth factors or hormones. The “classical” p21 ras family of mammalian proto-oncogenes consisting of Harvey-ras (Ha-ras), Kirsten-ras (Ki-ras) 4a and 4b and Neuroblastoma-ras (N-ras) are the most well known members of the rapidly expanding Ras superfamily of small GTPases. Several in vitro (and in vivo) studies have demonstrated that the Ras family of proto-oncogenes are involved in the induction of malignant transformation, see for example Chin et al., (1999) Nature 400, 468-472. Consequently, the p21 Ras family are regarded as important targets in development of anticancer drugs and it has been found that the Ras proteins are either over-expressed or mutated (often leading to constitutively active Ras proteins) in approximately 25% of all human cancers. Interestingly, the ras gene mutations in most cancer types are frequently limited to only one of the ras genes and are dependent on tumour type and tissue. Ha-ras oncogenic activating mutations have been identified at codon 12, 13 and 61. Activating mutations in the Ha-ras gene are mainly restricted to thyroid, kidney, urinary tract and bladder cancer, while Ha-ras over-expression has been detected primarily in breast and colon cancer. Because of the evidence of ras involvement in cancer development, interruption of the ras pathway has been a major focus for drug development. Efforts have been concentrated on either inhibiting ras maturation and membrane localization or by inhibiting ras protein expression.

As specific inhibition of ras isoforms at the protein level has proven difficult due to amino acid sequence homology, inhibition of protein expression by specific targeting of ras isoforms at the mRNA level has been attempted using ribozymes, antisense encoding vectors and antisense oligonucleotides.

Several studies have been published showing tumour growth inhibition in xenograft mouse models treated with antisense oligonucleotides targeted to Ha-ras. Gray et al. (1993) Cancer Research 53, 577-580 showed inhibition of tumour growth of oncogenic Ha-ras transformed NIH-3T3 cells pretreated with antisense oligonucleotides targeting an intron in the 5′ UTR of the Ha-ras mRNA. Using a similar model, Wickstrom et al. (1997), Oligonucleotides as Therapeutic Agents, Wiley, London, 124-141, showed 80% inhibition of tumour growth of oncogenic Ha-ras transformed NIH-3T3 cells treated by subcutaneous injection of antisense ODN targeting Ha-ras codon 12 mutation.

Schwab et al. (1994) Proceedings of the National Academy of Science 91, 10640-10464 investigated the effect of phosphorothioate oligonucleotides bound to nano-particles on oncogenic Ha-ras transformed cell lines in vitro and in vivo. Particle-bound antisense oligonucleotides targeting Ha-ras codon 12 mutation showed a 5-fold decrease in tumour growth compared to an inverse sequence control oligonucleotide when administered by intra-tumoral injection.

An antisense phosphorothioate oligo targeted to the AUG start codon of Ha-Ras (ISIS 2503) developed by Isis Pharmaceuticals has shown potent Ha-ras downregulation in vitro and tumour growth inhibition in human tumour xenografts in vivo. This antisense oligo was selected as the most potent inhibitor of ras mRNA assayed by Northern blot and it was shown to have an IC50=45 nM (Bennett et al. (1996) Antisense Therapeutics, Humana Press, Totowa, N.J., 13-47).

Interestingly, the anti-tumour effect of the ISIS 2503 Ha-ras antisense oligo in mouse models was not limited to Ha-ras mutated xenografts, but also showed tumour growth inhibition in Ki-ras mutated tumour xenografts (Cowsert (1997) Anti-Cancer Drug Design 12, 359-371).

Modification of ISIS 2503 with second-generation compounds conferring enhanced affinity and nuclease resistance has been shown to significantly improve the antisense effect. Incorporation of 2′-methoxyethyl (MOE) into ISIS 2503 increased the potency (IC50=14,7 nM) and the duration of antisense effect in vitro (Cowsert (1997) Anti-Cancer Drug Design 12, 359-371). ISIS 2503 is currently in phase I/II clinical trials either alone or in combination with chemotherapeutic agents against a variety of advanced cancers.

Casey-Cunningham et al. (2001) Cancer 92, 1265-1271, reported that in a phase I study of ISIS 2503 in advanced carcinoma, the compound was well tolerated but none of the 23 patients showed either complete or partial response. However, 4 patients had stable disease for 2 months or longer.

The above-mentioned phosphorothioate and MOE antisense compounds, typically between 20 and 25 base pairs, have been described in several patent applications (WO9222651, WO9408003, WO9428720, WO9849349, WO9902732, WO99227723). However, all disclosed compounds are targeted to two sites on Ha-ras, namely the codon 12 mutation or the AUG start codon, which only constitute a very small portion of the whole target. The codon 12 mutation is also targeted by one antisense sequence disclosed in WO98500540, which is tested with different phosphorothioate contents.

U.S. Pat. No. 6,117,848 discloses a few Ki-ras antisense oligonucleotides based on phosphorothidate chemistry or O′-2-methyl and in U.S. Pat. No. 5,872,242 a few N-ras phosphorothioate oligonucleotides were disclosed.

U.S. Pat. No. 5,874,416 discloses a single 26-mer antisense oligonucleotide targeted to a portion of the 5′-UTR region where all cytosine bases in CG dinucleotide pairs are 5-methylcytosine.

Most of the oligonucleotides currently in clinical trials are based on the phosphorothioate chemistry from 1988, which was the first useful antisense chemistry to be developed. However, as it has become clear in recent years this chemistry has serious shortcomings that limit its clinical use. These include low affinity for their target mRNA, which negatively affects potency and puts restrictions on how small active oligonucleotides can be thus complicating manufacture and increasing treatment costs. Also, their low affinity translate into poor accessibility to the target mRNA thus complicating identification of active compounds. Finally, phosphorothioate oligonucleotides suffer from a range of side effects that narrow their therapeutic window.

To deal with these and other problems much effort has been invested in creating novel analogues with improved properties. As depicted in the scheme 1 below, these include wholly artificial analogues such as PNA and Morpholino and more conventional DNA analogues such as boranosphosphates, N3′-P5′phosphoroamidates and several 2′ modified analogues, such as 2′-F, 2′-O-Me, 2′-O-methoxyethyl (MOE) and 2′-O-(3-aminopropyl) (AP). More recently hexitol nucleic acid (HNA), 2′-F-arabino nucleic acid (2′-F-ANA) and D-cyclohexenyl nucleoside (CeNA) have been introduced.

Many of these analogues exhibit improved binding to complementary nucleic acids, improvements in bio-stability or they retain the ability to recruit a cellular enzyme, RNAseH, which is involved in the mode-of-action of many antisense compounds. None of them, however, combine all of these advantages and in many cases improvements in one of the properties compromise one or more of the other properties. Also, in many cases new complications have been noted which seriously limits the commercial value of some of the analogues. These include low solubility, complex oligomerisation chemistries, very low cellular up-take, incompatibility with other chemistries, etc. For example, the MOE chemistry has several limitations. It has only modest affinity, which only manifests when several MOE's are inserted en block into the oligo. MOE belongs to the family of 2′-modifications and it is well known, for this group of compound, that the antisense activity is directly correlated with RNA binding affinity in vitro. A MOE 20 bp gapmer (5MOE/PO-10OPS-5MOE/PO) targeting c-raf has been reported to have an IC₅₀ of about 20 nm in T24 cells and an MOE gapmer targeting PKC-a has been reported to have an IC₅₀ of 25 nm in A549 cells. In comparison, phosphorthioate compounds used in antisense experiments typically exhibit IC₅₀ in the 150 nm range. (Stein, Kreig, Applied Antisense Oligonucleotide Technology, Wiley-Liss, 1988, p 87-90)

It is a principal object of the present invention to provide novel oligomeric compounds, against the Ha-ras mRNA. The compounds of the invention have been found to exhibit an decreased IC₅₀ (thus increased activity), thereby facilitating an effective treatment of a variety of cancer diseases in which the expression of Ha-ras is implied as a causative or related agent. As explained in the following, this objective is best achieved through the utilisation of a super high affinity chemistry termed LNA (Locked Nucleic Acid).

The present invention is directed to oligomeric compounds, particularly LNA antisense oligonucleotides, which are targeted to a nucleic acid encoding Ha-ras and which modulate the expression of the Ha-ras. This modulation was particularly a very potent down regulation Ha-ras mRNA as well as elicitation of apoptotic response. The LNA-containing oligomeric compounds can be as low as an 8-mer and certainly highly active as a 16-mers, which is considerably shorter than the reported antisense compounds targeting Ha-ras. These 16-mer oligomeric compounds have an IC₅₀ in the sub-nanomolar range. The invention enables a considerable shortening of the usual length of an antisense oligomers (from 20-25 mers to, e.g., 8-16 mers) without compromising the affinity required for pharmacological activity. As the intrinsic specificity of an oligo is inversely correlated to its length, such a shortening will significantly increase the specificity of the antisense compound towards its RNA target. Furthermore, it is anticipated that shorter oligomeric compounds have a higher biostability and cell permeability than longer oligomeric compounds. For at least these reasons, the present invention is a considerable contribution to the art.

### SUMMARY OF THE INVENTION

The present invention is directed to oligomeric compounds, particularly LNA antisense oligonucleotides, which are targeted to a nucleic acid encoding the ras family of proto-oncogenes, preferably Ha-ras, Ki-ras and N-ras, most preferably Ha-ras, and which modulate the expression of the ras. Pharmaceutical and other compositions comprising the oligomeric compounds of the invention are also provided.

A central aspect of the invention to provide a compound consisting of from 8-50 nucleosides, wherein said compound comprises a subsequence of at least 8 nucleosides, said subsequence being located within a sequence selected those listed in Table 1 and 4.

Further provided are methods of modulating the expression of ras in cells or tissues comprising contacting said cells or tissues with one or more of the oligomeric compounds or compositions of the invention. Also disclosed are methods of treating an animal or a human, suspected of having or being prone to a disease or condition, associated with expression of ras by administering a therapeutically or prophylactically effective amount of one or more of the oligomeric compounds or compositions of the invention. Further, methods of using oligomeric compounds for the inhibition of expression of ras and for treatment of diseases associated with ras activity are provided. Examples of such diseases are different types of cancer, such as for instance lung, breast, colon, prostate, pancreas, lung, liver, thyroid, kidney, brain, testes, stomach, intestine, bowel, spinal cord, sinuses, bladder, urinary tract or ovaries.

### BRIEF DESCRIPTION OF THE FIGURES

FIG. 1. Illustration of the different designs of the invention: Gapmers, Head- and Tailmers and Mixmers of different composition. For the mixmer, the numbers designate the alternate contiguous stretch of DNA, β-D-oxy-LNA or α-L-LNA. In the drawing, the line is DNA, the gray shadow corresponds to α-L-LNA residues and the rectangle is β-D-oxy-LNA.

FIG. 2 illustrates potency and specificity of LNA oligomeric compounds in an in vitro system. The LNA 16-mers shows effective down regulation, much better than the phosphorthioate 20-mer. The LNA oligomeric compounds also shows good specificity, compared to the compounds containing 6 mismatches. (The 4% given in italic have a 28S background smear. This leads to an overestimate of the 28S signal intensity. Therefore the % mRNA is put in brackets on the left side and not corrected for the RNA loading (i.e. the 285 signal).

FIG. 3 shows tumor growth reduction by the oligomeric compound Cur2524 (LNA-gapmer). It is also shown that the iso-sequential 16-mer phosphorothioate and the mismatch control did not have any effect.

FIG. 4 illustrates that the 16-mer LNA oligomeric compound Cur 2131 is more potent than the benchmark compound, ISIS2503, here called Cur2119, which is a phosphorthioate 20-mer. The in vivo model was 15PC3 tumour growth inhibition in nude mice treated with 1 mg/kg/day of the oligomeric compounds for 14 days administered continuously by Alzet osmotic pumps.

FIG. 5 General scheme of the synthesis of thio LNA

FIG. 6 Upper panel antisense inhibition of Ha-ras with oligomeric compound CUR2713 induese apoptosis tested at 5 and 100 nM in duplicate from two seperat experoments. Lower panel antisense inhibition of Ha-ras with oligomeric compound CUR 2742, CUR2749, CUR2776 and CUR2778 at 100 nM induces apoptosis.

FIG. 7 SEQ ID No 1 GenBank accession number J00277

FIG. 8 shows that the vivo potency of alpha-L-oxy-LNA oligomeric compounds are at least as good as the beta-D-oxy LNA oligomeric compounds in a 15PC3 and a Miapaca tumor nude mice model dosing 1 mg/kg/day and 2.5 mg/kg/day. Numbers refer to internal “Cur” numbers.

FIG. 9 shows that the beta-D-oxy LNA oligomeric compounds 2713 and 2722 are potent inhibitors or tumor growth dosing 5 mg/kg/day in a Miapaca and 15PC3 nude mice model. Numbers refer to internal “Cur” numbers.

FIG. 10 shows that alpha-L-oxy LNA and beta-D-oxy LNA oligomeric compounds targeting Ha-ras show low toxicity in mice. Numbers refer to internal “Cur” numbers”

### DESCRIPTION OF THE INVENTION

The present invention employs oligomeric compounds, particularly antisense oligonucleotides, for use in modulating the function of nucleic acid molecules encoding the ras family of proto-oncogenes, preferably Ha-ras, Ki-ras and N-ras, most preferably Ha-ras. The modulation is ultimately a change in the amount of ras produced. In one embodiment this is accomplished by providing antisense compounds, which specifically hybridise with nucleic acids encoding Ha-ras. The modulation is preferably an inhibition of the expression of Ha-ras, which leads to a decrease in the number of functional proteins produced.

A first aspect of the invention relates to a compound consisting of a total of 8-50 nucleotides and/or nucleotidee analogues, wherein said compound comprises a subsequence of at least 8 nucleotides or nucleotide analogues, said subsequence being located within a sequence selected from the group consisting of SEQ ID NOS: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75.

In the present context, the term “nucleoside” is used in its normal meaning, i.e. it contains a 2-deoxyribose unit or a ribose unit which is bonded through its number one carbon atom to one of the nitrogenous bases adenine (A), cytosine (C), thymine (T), uracil (U) or guanine (G).

In a similar way, the term “nucleotide” means a 2-deoxyribose unit or RNA unit which is bonded through its number one carbon atom to one of the nitrogenous bases adenine (A), cytosine (C), thymine (T) or guanine (G), uracil (U) and which is bonded through its number five carbon atom to an internucleoside phosphate group, or to a terminal group.

When used herein, the term “nucleotide analogue” refers to a non-natural occurring nucleotide wherein either the ribose unit is different from 2-deoxyribose or RNA and/or the nitrogenous base is different from A, C, T and G and/or the internucleoside phosphate linkage group is different. Specific examples of nucleoside analogues are described by e.g. Freier & Altmann; Nucl. Acid Res., 1997, 25, 4429-4443 and Uhlmann; Curr. Opinion in Drug Development, 2000, 3(2), 293-213.

The terms “corresponding nucleoside analogue” and “corresponding nucleoside” are intended to indicate that the nucleobase in the nucleoside analogue and the nucleoside is identical. For example, when the 2-deoxyribose unit of the nucleotide is linked to an adenine, the “corresponding nucleoside analogue” contains a pentose unit (different from 2-deoxyribose) linked to an adenine.

The term “nucleic acid” is defined as a molecule formed by covalent linkage of two or more nucleotides. The terms “nucleic acid” and “polynucleotide” are used interchangeable herein

The term “nucleic acid analogue” refers to a non-natural nucleic acid binding compound.

Nucleotide analogues and nucleic acid analogues are described in e.g. Freier & Altmann (Nucl. Acid Res., 1997, 25, 4429-4443) and Uhlmann (Curr. Opinion in Drug & Development (2000, 3(2): 293-213). Scheme 1 illustrates selected examples of nucleotide analogues suitable for making nucleic acids.

The term “LNA” refers to a nucleotide containing one bicyclic nucleoside analogue, also referred to as a LNA monomer, or an oligonucleotide containing one or more bicyclic nucleoside analogues. LNA monomers are described in WO 9914226 and subsequent applications, WO0056746, WO0056748, WO0066604, WO00125248, WO0228875, WO2002094250 and PCT/DK02/00488. One particular example of a thymidine LNA monomer is the (1S, 3R, 4R, 7S)-7-hydroxy-1-hydroxymethyl-5-methyl-3-(thymin-1-yl)-2,5-dioxa-bicyclo[2:2:1]heptane.

The term “oligonucleotide” refers, in the context of the present invention, to an oligomer (also called oligo) or nucleic acid polymer (e.g. ribonucleic acid (RNA) or deoxyribonucleic acid (DNA)) or nucleic acid analogue of those known in the art, preferably Locked Nucleic Acid (LNA), or a mixture thereof. This term includes oligonucleotides composed of naturally occurring nucleobases, sugars and internucleoside (backbone) linkages as well as oligonucleotides having non-naturally-occurring portions which function similarly or with specific improved functions. A fully or partly modified or substituted oligonucleotides are often preferred over native forms because of several desirable properties of such oligonucleotides such as for instance, the ability to penetrate a cell membrane, good resistance to extra- and intracellular nucleases, high affinity and specificity for the nucleic acid target. The LNA analogue is particularly preferred exhibiting the above-mentioned properties.

By the term “unit” is understood a monomer.

The term “at least one” comprises the integers larger than or equal to 1, such as 1, 2, 3, 4, 5, 6, 7, 8, 9, 10, 11, 12, 13, 14, 15, 16, 17 and so forth.

The term “thio-LNA” comprises a locked nucleotide in which at least one of X or Y in Scheme 2 is selected from S or —CH₂—S—. Thio-LNA can be in both beta-D and alpha-L-configuration.

The term “amino-LNA” comprises a locked nucleotide in which at least one of X or Y in Scheme 2-N(H)—, N(R)—, CH₂—N(H)—, —CH₂—N(R)— where R is selected form hydrogen and C₁₋₄-alkyl. Amino-LNA can be in both beta-D and alpha-L-configuration.

The term “oxy-LNA” comprises a locked nucleotide in which at least one of X or Y in Scheme 2 represents —O— or —CH₂—O—. Oxy-LNA can be in both beta-D and alpha-L-configuration.

The term “ena-LNA” comprises a locked nucleotide in which Y in Scheme 2 is —CH₂—O—.

By the term “alpha-L-LNA” comprises a locked nucleotide represented as shown in Scheme 3 (structure to the right).

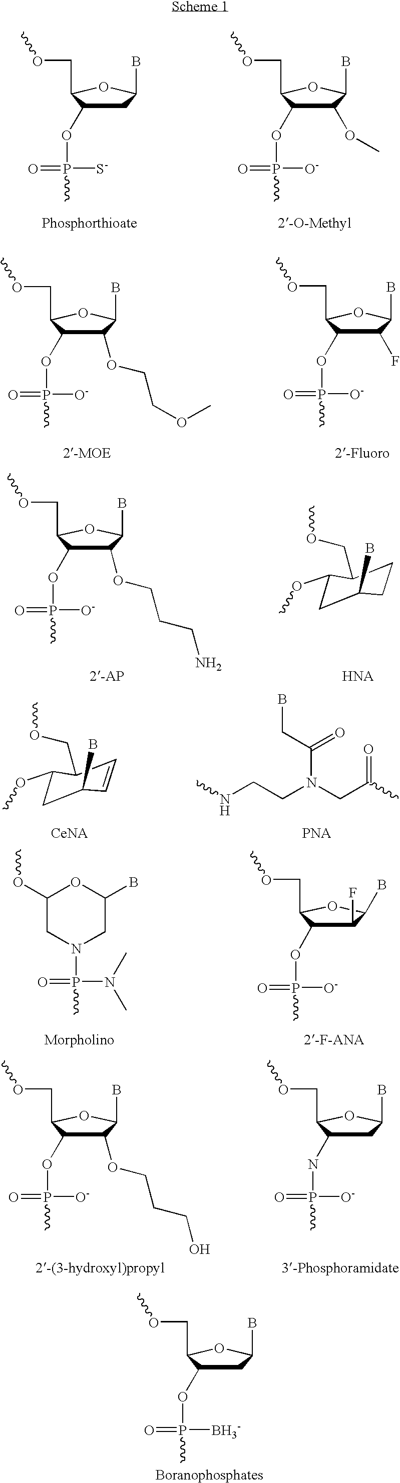

By the term “LNA derivatives” comprises all locked nucleotide in Scheme 2 as well as beta-D-methylene LNA e.g. thio-LNA, amino-LNA, alpha-L-oxy-LNA and ena-LNA.

The term “linkage group” is intended to mean a group capable of covalently coupling together two nucleosides, two nucleoside analogues, a nucleoside and a nucleoside analogue, etc. Specific and preferred examples include phosphate groups and phosphorothioate groups.

In the present context the term “conjugate” is intended to indicate a heterogenous molecule formed by the covalent attachment of a compound as described herein (i.e. a compound comprising a sequence of nucleosides or nucleoside analogues) to one or more non-nucleotide or non-polynucleotide moieties. Examples of non-nucleotide or non-polynucleotide moieties include macromolecular agents such as proteins, fatty acid chains, sugar residues, glycoproteins, polymers, or combinations thereof. Typically proteins may be antibodies for a target protein. Typical polymers may be polyethelene glycol.

The term “carcinoma” is intended to indicate a malignant tumor of epithelial origin. Epithelial tissue covers or lines the body surfaces inside and outside the body. Examples of epithelial tissue are the skin and the mucosa and serosa that line the body cavities and internal organs, such as intestines, urinary bladder, uterus, etc. Epithelial tissue may also extend into deeper tissue layers to from glands, such as mucus-secreting glands.

The term “sarcoma” is intended to indicate a malignant tumor growing from connective tissue, such as cartilage, fat, muscles, tendons and bones.

The term “glioma”, when used herein, is intended to cover a malignant tumor originating from glial cells

The term “a” as used about a nucleoside, a nucleoside analogue, a SEQ ID NO, etc. is intended to mean one or more. In particular, the expression “a component (such as a nucleoside, a nucleoside analogue, a SEQ ID NO or the like) selected from the group consisting of . . . ” is intended to mean that one or more of the cited components may be selected. Thus, expressions like “a component selected from the group consisting of A, B and C” is intended to include all combinations of A, B and C, i.e. A, B, C, A+B, A+C, B+C and A+B+C.

In the present context, the term “C₁₋₄-alkyl” is intended to mean a linear or branched saturated hydrocarbon chain wherein the chain has from one to four carbon atoms, such as methyl, ethyl, n-propyl, isopropyl, n-butyl, isobutyl, sec-butyl and tert-butyl.

As used herein, the terms “target nucleic acid” encompass DNA encoding the survivin, RNA (including pre-mRNA and mRNA) transcribed from such DNA, and also cDNA derived from such RNA.

As used herein, the term “gene” means the gene including exons, introns, non-coding 5′ and 3′ regions and regulatory elements and all currently known variants thereof and any further variants, which may be elucidated.

As used herein, the terms “oligomeric compound” refers to an oligonucleotide which can induce a desired therapeutic effect in humans through for example binding by hydrogen bonding to either a target gene “Chimeraplast” and “TFO”, to the RNA transcript(s) of the target gene “antisense inhibitors”, “siRNA”, “ribozymes” and oligozymes” or to the protein(s) encoding by the target gene “aptamer”, spiegelmer” or “decoy”.

As used herein, the term “mRNA” means the presently known mRNA transcript(s) of a targeted gene, and any further transcripts, which may be identified.

As used herein, the term “modulation” means either an increase (stimulation) or a decrease (inhibition) in the expression of a gene. In the present invention, inhibition is the preferred form of modulation of gene expression and mRNA is a preferred target.

As used herein, the term “targeting” an antisense compound to a particular target nucleic acid means providing the antisense oligonucleotide to the cell, animal or human in such a way that the antisense compound are able to bind to and modulate the function of its intended target.

As used herein, “hybridisation” means hydrogen bonding, which may be Watson-Crick, Holstein, reversed Holstein hydrogen bonding, etc. between complementary nucleoside or nucleotide bases. Watson and Crick showed approximately fifty years ago that deoxyribo nucleic acid (DNA) is composed of two strands which are held together in a helical configuration by hydrogen bonds formed between opposing complementary nucleobases in the two strands. The four nucleobases, commonly found in DNA are guanine (G), adenine (A), thymine (T) and cytosine (C) of which the G nucleobase pairs with C, and the A nucleobase pairs with T. In RNA the nucleobase thymine is replaced by the nucleobase uracil (U), which similarly to the T nucleobase pairs with A. The chemical groups in the nucleobases that participate in standard duplex formation constitute the Watson-Crick face. Hoogsteen showed a couple of years later that the purine nucleobases (G and A) in addition to their Watson-Crick face have a Hoogsteen face that can be recognised from the outside of a duplex, and used to bind pyrimidine oligonucleotides via hydrogen bonding, thereby forming a triple helix structure.

In the context of the present invention “complementary” refers to the capacity for precise pairing between two nucleotides or nucleoside sequences with one another. For example, if a nucleotide at a certain position of an oligonucleotide is capable of hydrogen bonding with a nucleotide at the corresponding position of a DNA or RNA molecule, then the oligonucleotide and the DNA or RNA are considered to be complementary to each other at that position. The DNA or RNA and the oligonucleotide are considered complementary to each other when a sufficient number of nucleotides in the oligonucleotide can form hydrogen bonds with corresponding nucleotides in the target DNA or RNA to enable the formation of a sTable complex. To be stable in vitro or in vivo the sequence of an antisense compound need not be 100% complementary to its target nucleic acid. The terms “complementary” and “specifically hybridisable” thus imply that the antisense compound binds sufficiently strongly and specifically to the target molecule to provide the desired interference with the normal function of the target whilst leaving the function of non-target mRNAs unaffected.

Antisense and other oligomeric compounds of the invention, which modulate expression of the target, are identified through experimentation or though rational design based on sequence information on the target and know-how on how best to design an oligomeric compound against a desired target. The sequences of these compounds are preferred embodiments of the invention. Likewise, the sequence motifs in the target to which these preferred oligomeric compounds are complementary (referred to as “hot spots”) are preferred sites for targeting.

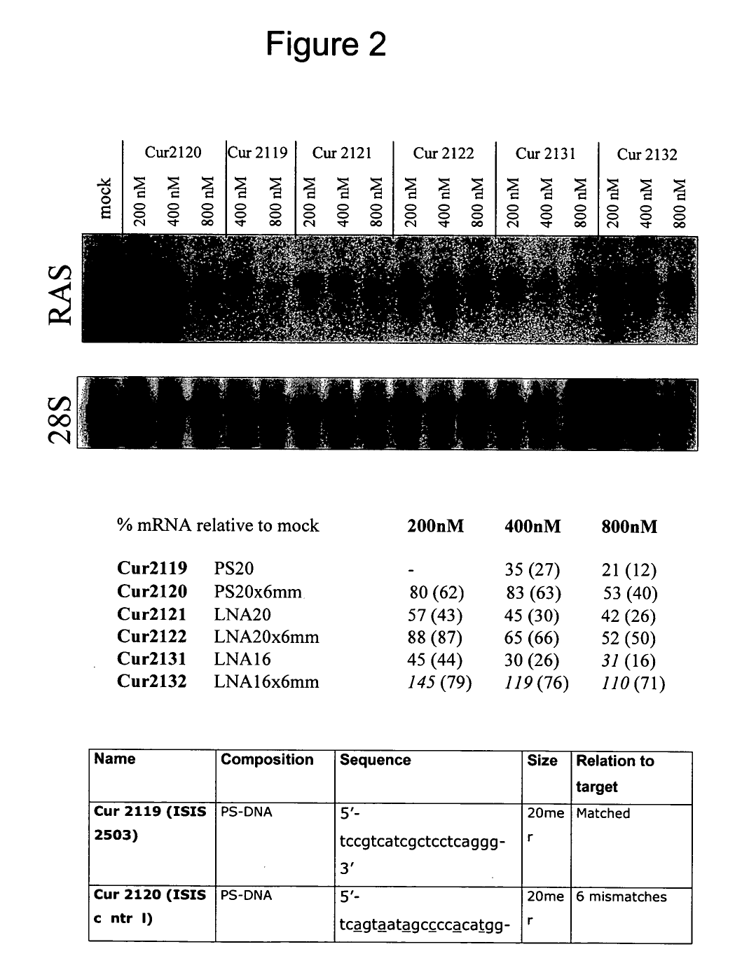

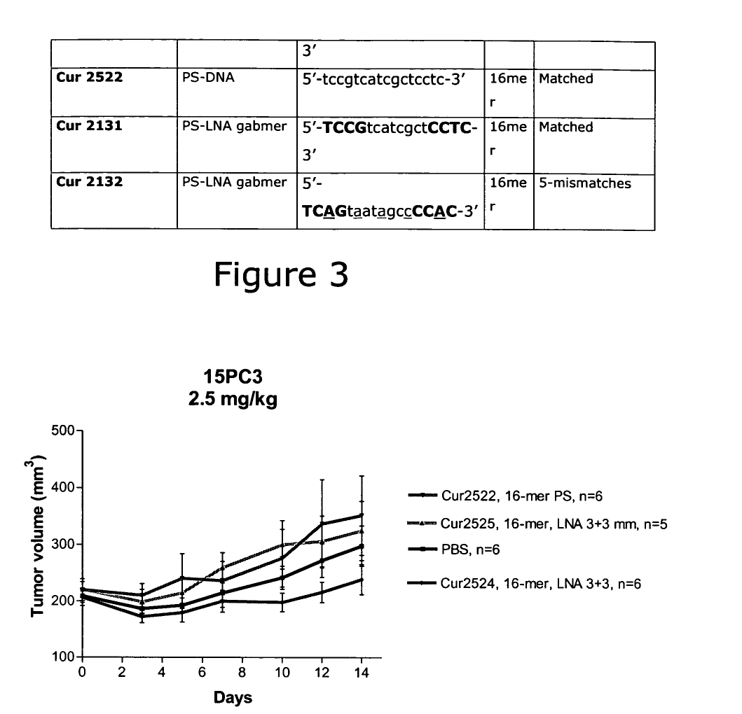

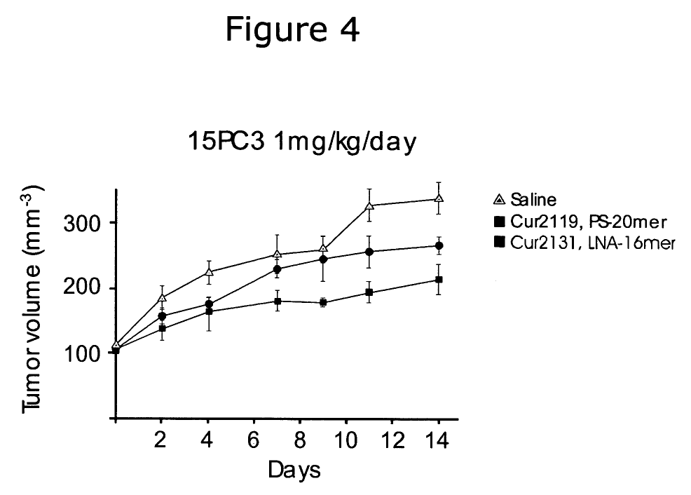

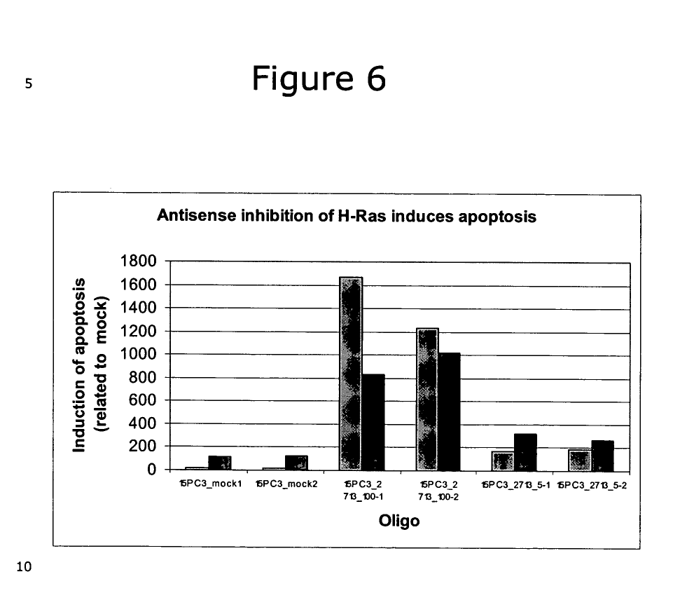

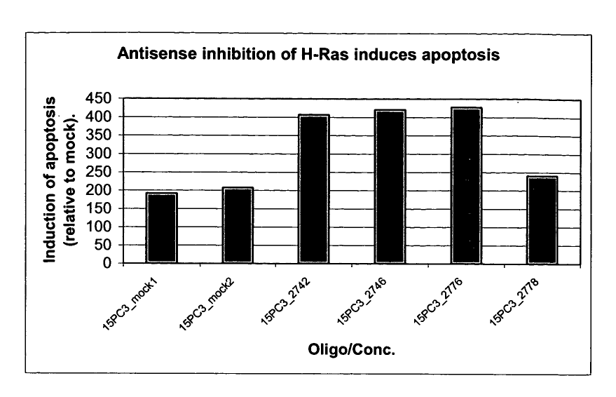

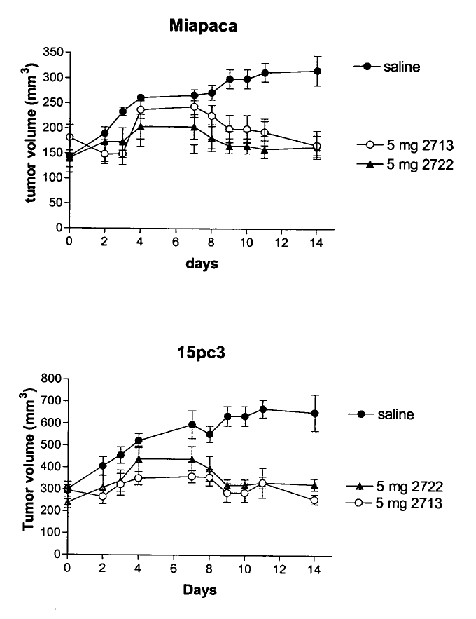

Preferred oligomeric compounds comprises at least a 8-nucleobase portion, said subsequence being selected from SEQ ID NO 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 76, 77 or 79 and their sequences are presented in table 1, 3 and 4. The oligomeric compounds according to the invention are potent modulators of target. For example, in vitro inhibition of target is shown in Table 1 measured by Real time PCR. FIG. 2 shows in vitro potency and specificity of oligomeric compounds according to the invention measured by Northern Blot. Very low IC50 of oligomeric compounds is shown in table 2 (compared to the previously reported IC50, see section “Background of the invention”). The compound of the invention also induces apoptosis (FIG. 6). In vivo specificity and potency of oligomeric compounds are shown in FIG. 3. Furthermore, in vivo superiority of a short oligomeric compound compared to a traditional long antisense compound is shown FIG. 4. FIG. 9 show in vivo potency of 2 compounds of the invention. All the above-mentioned experimental observations show that the compounds according to the invention can constitute the active compound in a pharmaceutical composition.

In one embodiment the nucleobase portion is selected from at least 9, least 10, least 11, least 12, least 13, least 14 and least 15.

Compounds of the invention are shown in table 1, 3, 4 and 5.

Preferred oligomeric compounds according to the invention are SEQ ID NO 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 and 75.

In another embodiment of the invention, said nucleosides are linked to each other by means of a phosphorothioate group. An interesting embodiment of the invention is directed to compounds of SEQ NO 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 and 75 wherein each linkage group within each compound is a phosphorothioate group. Such modifications is denoted by the subscript S. Alternatively stated, one aspect of the invention is directed to compounds of SEQ NO 2_{A}, 3_{A}, 4_{A}, 5_{A}, 6_{A}, 7_{A}, 8_{A}, 9_{A}, 10_{A}, 11_{A}, 12_{A}, 13_{A}, 14_{A}, 15_{A}, 16_{A}, 17_{A}, 18_{A}, 19_{A}, 20_{A}, 21_{A}, 22_{A}, 23_{A}, 24_{A}, 25_{A}, 26_{A}, 27_{A}, 28_{A}, 29_{A}, 30_{A}, 31_{A}, 32_{A}, 33_{A}, 34_{A}, 35_{A}, 36_{A}, 37_{A}, 38_{A}, 39_{A}, 40_{A}, 41_{A}, 42_{A}, 43_{A}, 44_{A}, 45_{A}, 46_{A}, 47_{A}, 48_{A}, 49_{A}, 50_{A}, 51_{A}, 52_{A}, 53_{A}, 54_{A}, 55_{A}, 56_{A}, 57_{A}, 58_{A}, 59_{A}, 60_{A}, 61_{A}, 62_{A}, 63_{A}, 64_{A}, 65_{A}, 66_{A}, 67_{A}, 68_{A}, 69_{A}, 70_{A}, 71_{A}, 72_{A}, 73_{A}, 74_{A}, and 75_{A}.

A further aspect of the invention is directed to compounds of SEQ NO 2_{B}, 3_{B}, 4_{B}, 5_{B}, 6_{S}, 7_{S}, 8_{B}, 9_{B}, 10_{B}, 11_{B}, 12_{B}, 13_{B}, 14_{B}, 15_{B}, 16_{B}, 17_{B}, 18_{B}, 19_{B}, 20_{B}, 21_{B}, 22_{B}, 23_{B}, 24_{B}, 25_{B}, 26_{B}, 27_{B}, 28_{B}, 29_{B}, 30_{B}, 31_{B}, 32_{B}, 33_{B}, 34_{B}, 35_{B}, 36_{B}, 37_{S}, 38_{B}, 39_{B}, 40_{B}, 41_{B}, 42_{B}, 43_{B}, 44_{B}, 45_{B}, 46_{B}, 47_{B}, 48_{B}, 49_{B}, 50_{B}, 51_{B}, 52_{B}, 53_{B}, 54_{B}, 55_{B}, 56_{B}, 57_{B}, 58_{B}, 59_{B}, 60_{B}, 61_{B}, 62_{B}, 63_{B}, 64_{B}, 65_{B}, 66_{B}, 67_{B}, 68_{B}, 69_{B}, 70_{B}, 71_{B}, 72_{B}, 73_{B}, 74_{B}, and 75_{B}.

A further aspect of the invention is directed to compounds of SEQ NO 2_{C}, 3_{C}, 4_{C}, 5_{C}, 6_{S}, 7_{S}, 8_{C}, 9_{C}, 10_{C}, 11_{C}, 12_{C}, 13_{C}, 14_{C}, 15_{C}, 16_{C}, 17_{C}, 18_{C}, 19_{C}, 20_{C}, 21_{C}, 22_{C}, 23_{C}, 24_{C}, 25_{C}, 26_{C}, 27_{C}, 28_{C}, 29_{C}, 30_{C}, 31_{C}, 32_{C}, 33_{C}, 34_{C}, 35_{C}, 36_{C}, 37_{S}, 38_{C}, 39_{C}, 40_{C}, 41_{C}, 42_{C}, 43_{C}, 44_{C}, 45_{C}, 46_{C}, 47_{C}, 48_{C}, 49_{C}, 50_{C}, 51_{C}, 52_{C}, 53_{C}, 54_{C}, 55_{C}, 56_{C}, 57_{C}, 58_{C}, 59_{C}, 60_{C}, 61_{C}, 62_{C}, 63_{C}, 64_{C}, 65_{C}, 66_{C}, 67_{C}, 68_{C}, 69_{C}, 70_{C}, 71_{C}, 72_{C}, 73_{C}, 74_{C}, and 75_{C}.

In one embodiment of the invention the oligomeric compounds are containing at least on unit of chemistry termed LNA (Locked Nucleic Acid).

LNA monomer typically refers to a bicyclic nucleoside analogue, as described in the International Patent Application WO 99/14226 and subsequent applications, WO056746, WO0056748, WO0066604, WO00125248, WO0228875, WO2002094250 and PCT/DK02/00488 all incorporated herein by reference. Preferred LNA monomers structures are exemplified in Scheme 2

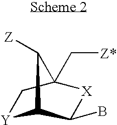

wherein X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— CH₂— or —CH₂—CH— (if part of a double bond), —CH═CH—, where R is selected form hydrogen and C₁₋₄-alkyl. The asymmetric groups may be found in either orientation.

In Scheme 2, the 4 chiral centers are shown in a fixed configuration. However, the configuarations in Scheme 2 are not necessarily fixed. Also comprised in this invention are compounds of the general Scheme 2 in which the chiral centers are found in different configurations, such as those represented in Scheme 3 or 4. Thus, the intention in the illustration of Scheme 2 is not to limit the configuration of the chiral centre. Each chiral center in Scheme 2 can exist in either R or S configuration. The definition of R (rectus) and S (sinister) are described in the IUPAC 1974 Recommendations, Section E, Fundamental Stereochemistry: The rules can be found in Pure Appl. Chem. 45, 13-30, (1976) and in “Nomenclature of organic Chemistry” pergamon, New York, 1979.

Z and Z* are independently absent, selected among an internucleoside linkage, a terminal group or a protecting group

The internucleoside linkage may be —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, —NR^{H}—CO—NR^{H}—, —O—CO—O—, —O—CO—NR^{H}—, —NR^{H}—CO—CH₂—, —O—CH₂—CO—NR^{H}—, —O—CH₂—CH₂—NR^{H}—, —CO—NR^{H}—CH₂—, —CH₂—NR^{H}—CO—, —O—CH₂—CH₂—S—, —S—CH₂—CH₂—O—, —S—CH₂—CH₂—S—, —CH₂—SO₂—CH₂—, —CH₂—CO—NR^{H}—, —O—CH₂—CH₂—NR^{H}—CO—, —CH₂—NCH₃—O—CH₂—, where R^{H} is selected form hydrogen and C₁₋₄-alkyl,

The terminal groups are selected independently among from hydrogen, azido, halogen, cyano, nitro, hydroxy, Prot-O—, Act-O—, mercapto, Prot-S—, Act-S—, C₁₋₆-alkylthio, amino, Prot-N(R^{H})—, Act-N(R^{H})—, mono- or di(C₁₋₆-alkyl)amino, optionally substituted C₁₋₆-alkoxy, optionally substituted C₁₋₆-alkyl, optionally substituted C₂₋₆-alkenyl, optionally substituted C₂₋₆-alkenyloxy, optionally substituted C₂₋₆-alkynyl, optionally substituted C₂₋₆-alkynyloxy, monophosphate, monothiophosphate, diphosphate, dithiophosphate triphosphate, trithiophosphate, DNA intercalators, photochemically active groups, thermochemically active groups, chelating groups, reporter groups, ligands, carboxy, sulphono, hydroxymethyl, Prot-O—CH₂—, Act-O—CH₂—, aminomethyl, Prot-N(R^{H})—CH₂—, Act-N(R^{H})—CH₂—, carboxymethyl, sulphonomethyl, where Prot is a protection group for —OH, —SH, and —NH(R^{H}), respectively, Act is an activation group for —OH, —SH, and —NH(R^{H}), respectively, and R^{H} is selected from hydrogen and C₁₋₆-alkyl;

The protection groups of hydroxy substituents comprises substituted trityl, such as 4,4′-dimethoxytrityloxy (DMT), 4-monomethoxytrityloxy (MMT), and trityloxy, optionally substituted 9-(9-phenyl)xanthenyloxy (pixyl), optionally substituted methoxytetrahydropyranyloxy (mthp), silyloxy such as trimethylsilyloxy (TMS), triisopropylsilyloxy (TIPS), tert-butyldimethylsilyloxy (TBDMS), triethylsilyloxy, and phenyldimethylsilyloxy, tert-butylethers, acetals (including two hydroxy groups), acyloxy such as acetyl or halogen substituted acetyls, e.g. chloroacetyloxy or fluoroacetyloxy, isobutyryloxy, pivaloyloxy, benzoyloxy and substituted benzoyls, methoxymethyloxy (MOM), benzyl ethers or substituted benzyl ethers such as 2,6-dichlorobenzyloxy (2,6-Cl₂Bzl). Alternatively when Z or Z* is hydroxyl they may be protected by attachment to a solid support optionally through a linker.

When Z or Z* is amino groups illustrative examples of the amino protection protections are fluorenylmethoxycarbonylamino (Fmoc), tert-butyloxycarbonylamino (BOC), trifluoroacetylamino, allyloxycarbonylamino (alloc, AOC), Z benzyloxycarbonylamino (Cbz), substituted benzyloxycarbonylaminos such as 2-chloro benzyloxycarbonylamino (2-CIZ), monomethoxytritylamino (MMT), dimethoxytritylamino (DMT), phthaloylamino, and 9-(9-phenyl)xanthenylamino (pixyl).

In the embodiment above, Act designates an activation group for —OH, —SH, and —NH(R^{H}), respectively. Such activation groups are, e.g., selected from optionally substituted O-phosphoramidite, optionally substituted O-phosphortriester, optionally substituted O-phosphordiester, optionally substituted H-phosphonate, and optionally substituted O-phosphonate.

In the present context, the term “phosphoramidite” means a group of the formula —P(OR^{x})—N(R^{y})₂, wherein R^{x} designates an optionally substituted alkyl group, e.g. methyl, 2-cyanoethyl, or benzyl, and each of R^{y} designate optionally substituted alkyl groups, e.g. ethyl or isopropyl, or the group —N(R^{y})₂ forms a morpholino group (—N(CH₂CH₂)₂O). R^{x} preferably designates 2-cyanoethyl and the two R^{y} are preferably identical and designate isopropyl. Thus, an especially relevant phosphoramidite is N,N-diisopropyl-O-(2-cyanoethyl)phosphoramidite.

B constitutes a natural or non-natural nucleobase and selected among adenine, cytosine, 5-methylcytosine, isocytosine, pseudoisocytosine, guanine, thymine, uracil, 5-bromouracil, 5-propynyluracil, 5-propyny-6-fluoroluracil, 5-methylthiazoleuracil, 6-aminopurine, 2-aminopurine, inosine, diaminopurine, 7-propyne-7-deazaadenine, 7-propyne-7-deazaguanine, and 2-chloro-6-aminopurine.

Particularly preferred bicyclic structures are shown in Scheme 3 below:

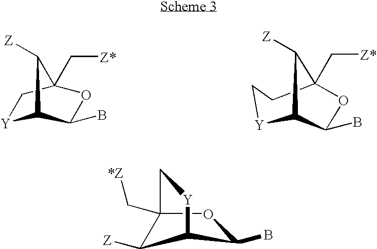

Where Y is —O—, —S—, —NH—, or N(R^{H}); Z and Z* are independently absent, selected among an internucleoside linkage, a terminal group or a protecting group. The internucleotide linkage may be —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

The terminal groups are selected independently among from hydrogen, azido, halogen, cyano, nitro, hydroxy, Prot-O—, Act-O—, mercapto, Prot-S—, Act-S—, C₁₋₆-alkylthio, amino, Prot-N(R^{H})—, Act-N(R^{H})—, mono- or di(C₁₋₆-alkyl)amino, optionally substituted C₁₋₆-alkoxy, optionally substituted C₁₋₆-alkyl, optionally substituted monophosphate, monothiophosphate, diphosphate, dithiophosphate triphosphate, trithiophosphate, where Prot is a protection group for —OH, —SH, and —NH(R^{H}), respectively, Act is an activation group for —OH, —SH, and —NH(R^{H}), respectively, and R^{H} is selected from hydrogen and C₁₋₆-alkyl.

The protection groups of hydroxy substituents comprises substituted trityl, such as 4,4′-dimethoxytrityloxy (DMT), 4-monomethoxytrityloxy (MMT), optionally substituted 9-(9-phenyl)xanthenyloxy (pixyl), optionally substituted methoxytetrahydropyranyloxy (mthp), silyloxy such as trimethylsilyloxy (TMS), triisopropylsilyloxy (TIPS), tert-butyl-dimethylsilyloxy (TBDMS), triethylsilyloxy, and phenyldimethylsilyloxy, tert-butylethers, acetals (including two hydroxy groups), acyloxy such as acetyl Alternatively when Z or Z* is hydroxyl they may be protected by attachment to a solid support optionally through a linker.

When Z or Z* is amino groups illustrative examples of the amino protection protections are fluorenylmethoxycarbonylamino (Fmoc), tert-butyloxycarbonylamino (BOC), trifluoroacetylamino, allyloxycarbonylamino (alloc, AOC), monomethoxytritylamino (MMT), dimethoxytritylamino (DMT), phthaloylamino.

In the embodiment above, Act designates an activation group for —OH, —SH, and —NH(R^{H}), respectively. Such activation groups are, e.g., selected from optionally substituted O-phosphoramidite, optionally substituted O-phosphortriester, optionally substituted O-phosphordiester, optionally substituted H-phosphonate, and optionally substituted O-phosphonate.

In the present context, the term “phosphoramidite” means a group of the formula —P(OR^{x})—N(R^{y})₂, wherein R^{x} designates an optionally substituted alkyl group, e.g. methyl, 2-cyanoethyl, and each of R^{y} designate optionally substituted alkyl groups, R^{x} preferably designates 2-cyanoethyl and the two R^{y} are preferably identical and designate isopropyl. Thus, an especially relevant phosphoramidite is N,N-diisopropyl-O-(2-cyanoethyl)-phosphoramidite.

B constitutes a natural or non-natural nucleobase and selected among adenine, cytosine, 5-methylcytosine, isocytosine, pseudoisocytosine, guanine, thymine, uracil, 5-bromouracil, 5-propynyluracil, 6-aminopurine, 2-aminopurine, inosine, diaminopurine, 2-chloro-6-aminopurine.

Specifically preferred LNA units are shown in scheme 4. B and Z* and Z as previously defined.

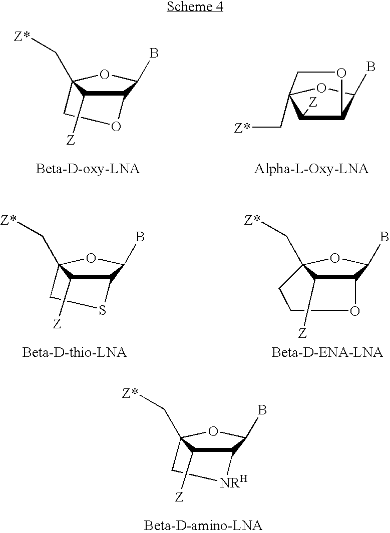

Therapeutic Principle

A person skilled in the art will appreciate that oligomeric compounds containing LNA can be used to combat ras linked diseases by many different principles, which thus falls within the spirit of the present invention.

For instance, LNA oligomeric compounds may be designed as antisense inhibitors, which are single stranded nucleic acids that prevent the production of a disease causing protein, by intervention at the mRNA level. Also, they may be designed as Ribozymes or Oligozymes which are antisense oligonucleotides which in addition to the target binding domain(s) comprise a catalytic activity that degrades the target mRNA (ribozymes) or comprise an external guide sequence (EGS) that recruit an endogenous enzyme (RNase P) which degrades the target mRNA (oligozymes)

Equally well, the LNA oligomeric compounds may be designed as siRNA's which are small double stranded RNA molecules that are used by cells to silence specific endogenous or exogenous genes by an as yet poorly understood “antisense-like” mechanism.

LNA oligomeric compounds may also be designed as Aptamers (and a variation thereof, termed Spiegelmers) which are nucleic acids that through intra-molecular hydrogen bonding adopt three-dimensional structures that enable them to bind to and block their biological targets with high affinity and specificity. Also, LNA oligomeric compounds may be designed as Decoys, which are small double-stranded nucleic acids that prevent cellular transcription factors from transactivating their target genes by selectively blocking their DNA binding site.

Furthermore, LNA oligomeric compounds may be designed as Chimeraplasts, which are small single stranded nucleic acids that are able to specifically pair with and alter a target gene sequence. LNA containing oligomeric compounds exploiting this principle therefore may be particularly useful for treating Ha-ras linked diseases that are caused by a mutation in the Ha-ras gene.

Finally, LNA oligomeric compounds may be designed as TFO's (triplex forming oligonucleotides), which are nucleic acids that bind to double stranded DNA and prevent the production of a disease causing protein, by intervention at the RNA transcription level.

Dictated in part by the therapeutic principle by which the oligonucleotide is intended to operate, the LNA oligomeric compounds in accordance with this invention preferably comprise from about 8 to about 60 nucleobases i.e. from about 8 to about 60 linked nucleosides. Particularly preferred compounds are antisense oligonucleotides comprising from about 12 to about 30 nucleobases and most preferably are antisense compounds comprising about 12-20 nucleobases.

Referring to the above principles by which an LNA oligomeric compound can elicit its therapeutic action the target of the present invention may be the Ha-ras gene, the mRNA or the protein. In the most preferred embodiment the LNA oligomeric compounds is designed as an antisense inhibitor directed against the Ha-ras pre-mRNA or Ha-ras mRNA.

The oligonucleotides may hybridize to any site along the Ha-ras pre-mRNA or mRNA such as sites in the 5′ untranslated leader, exons, introns and 3′ untranslated tail.

In a preferred embodiment, the oligonucleotide hybridizes to a portion of the human Ha-ras pre-mRNA or mRNA that comprises the translation-initiation site. More preferably, the Ha-ras oligonucleotide comprises a CAT sequence, which is complementary to the AUG initiation sequence of the Ha-ras pre-mRNA or RNA. In another embodiment, the Ha-ras oligonucleotide hybridizes to a portion of the splice donor site of the human Ha-ras pre-mRNA. In yet another embodiment, Ha-ras oligonucleotide hybridizes to a portion of the splice acceptor site of the human Ha-ras pre-mRNA. In another embodiment, the Ha-ras oligonucleotide hybridizes to portions of the human Ha-ras pre-mRNA or mRNA involved in polyadenylation, transport or degradation.

The skilled person will appreciate that preferred oligonucleotides are those that hybridize to a portion of the Ha-ras pre-mRNA or mRNA whose sequence does not commonly occur in transcripts from unrelated genes so as to maintain treatment specificity.

The oligomeric compound of the invention are designed to be sufficiently complementary to the target to provide the desired clinical response e.g. the oligomeric compound must bind with sufficient strength and specificity to its target to give the desired effect. In one embodiment, said compound modulating Ha-ras is designed so as to also modulate other specific nucleic acids which do not encode Ha-ras.

It is preferred that the oligomeric compound according to the invention is designed so that intra- and intermolecular oligonucleotide hybridisation is avoided.

In many cases the identification of an LNA oligomeric compound effective in modulating ras activity in vivo or clinically is based on sequence information on the target gene. However, one of ordinary skill in the art will appreciate that such oligomeric compounds can also be identified by empirical testing. As such Ha-ras oligomeric compounds having, for example, less sequence homology, greater or fewer modified nucleotides, or longer or shorter lengths, compared to those of the preferred embodiments, but which nevertheless demonstrate responses in clinical treatments, are also within the scope of the invention.

Antisense Drugs

In one embodiment of the invention the oligomeric compounds are suitable antisense drugs. The design of a potent and safe antisense drug requires the fine-tuning of diverse parameters such as affinity/specificity, stability in biological fluids, cellular uptake, mode of action, pharmacokinetic properties and toxicity.

Affinity & specificity: LNA with an oxymethylene 2′-O, 4′-C linkage (β-D-oxy-LNA), exhibits unprecedented binding properties towards DNA and RNA target sequences. Likewise LNA derivatives, such as amino-, thio- and α-L-oxy-LNA display unprecedented affinities towards complementary RNA and DNA and in the case of thio-LNA the affinity towards RNA is even better than with the β-D-oxy-LNA.

In addition to these remarkable hybridization properties, LNA monomers can be mixed and act cooperatively with DNA and RNA monomers, and with other nucleic acid analogues, such as 2′-O-alkyl modified RNA monomers. As such, the oligonucleotides of the present invention can be composed entirely of β-D-oxy-LNA monomers or it may be composed of β-D-oxy-LNA in any combination with DNA, RNA or contemporary nucleic acid analogues which includes LNA derivatives such as for instance amino-, thio- and α-L-oxy-LNA . The unprecedented binding affinity of LNA towards DNA or RNA target sequences and its ability to mix freely with DNA, RNA and a range of contemporary nucleic acid analogues has a range of important consequences according to the invention for the development of effective and safe antisense compounds.

Firstly, in one embodiment of the invention it enables a considerable shortening of the usual length of an antisense oligo (from 20-25 mers to, e.g., 12-15 mers) without compromising the affinity required for pharmacological activity. As the intrinsic specificity of an oligo is inversely correlated to its length, such a shortening will significantly increase the specificity of the antisense compound towards its RNA target. One embodiment of the invention is to, due to the sequence of the humane genome is available and the annotation of its genes rapidly progressing, identify the shortest possible, unique sequences in the target mRNA.

In another embodiment, the use of LNA to reduce the size of oligos significantly eases the process and prize of manufacture thus providing the basis for antisense therapy to become a commercially competitive treatment offer for a diversity of diseases.

In another embodiment, the unprecedented affinity of LNA can be used to substantially enhance the ability of an antisense oligo to hybridize to its target mRNA in-vivo thus significantly reducing the time and effort required for identifying an active compound as compared to the situation with other chemistries.

In another embodiment, the unprecedented affinity of LNA is used to enhance the potency of antisense oligonucleotides thus enabling the development of compounds with more favorable therapeutic windows than those currently in clinical trials.

When designed as an antisense inhibitor, the oligonucleotides of the invention bind to the target nucleic acid and modulate the expression of its cognate protein. Preferably, such modulation produces an inhibition of expression of at least 10% or 20% compared to the normal expression level, more preferably at least a 30%, 40%, 50%, 60%, 70%, 80%, or 90% inhibition compared to the normal expression level.

Typically, the LNA oligonucleotides of the invention will contain other residues than β-D-oxy-LNA such as native DNA monomers, RNA monomers, N3′-P5′ phosphoroamidates, 2′-F, 2′-O-Me, 2′-O-methoxyethyl (MOE), 2′-O-(3-aminopropyl) (AP), hexitol nucleic acid (HNA), 2′-F-arabino nucleic acid (2′-F-ANA) and D-cyclohexenyl nucleoside (CeNA). Also, the β-D-oxy-LNA-modified oligonucleotide may also contain other LNA units in addition to or in place of an oxy-LNA group. In particular, preferred additional LNA units include thio-LNA or amino-LNA monomers in either the D-β or L-α configurations or combinations thereof or ena-LNA. In general, an LNA-modified oligonucleotide will contain at least about 5, 10, 15 or 20 percent LNA units, based on total nucleotides of the oligonucleotide, more typically at least about 20, 25, 30, 40, 50, 60, 70, 80 or 90 percent LNA units, based on total bases of the oligonucleotide.

Stability in biological fluids: One embodiment of the invention includes the incorporation of LNA monomers into a standard DNA or RNA oligonucleotide to increase the stability of the resulting oligomeric compound in biological fluids e.g. through the increase of resistance towards nucleases (endonucleases and exonucleases). The extent of stability will depend on the number of LNA monomers used, their position in the oligonucleotide and the type of LNA monomer used. Compared to DNA and phosphorothioates the following order of ability to stabilize an oligonucleotide against nucleolytic degradation can be established: DNA<<phosphorothioates˜oxy-LNA<α-L-LNA<amino-LNA<thio-LNA.

Given the fact that LNA is compatible with standard DNA synthesis and mixes freely with many contemporary nucleic acid analogues nuclease resistance of LNA-oligomeric compounds can be further enhanced according to the invention by either incorporating other analogues that display increased nuclease stability or by exploiting nuclease-resistant internucleoside linkages e.g. phosphoromonothioate, phosphorodithioate, and methylphosphonate linkages, etc.

Mode of action: Antisense compounds according to the invention may elicit their therapeutic action via a variety of mechanisms and may be able to combine several of these in the same compound. In one scenario, binding of the oligonucleotide to its target (pre-mRNA or mRNA) acts to prevent binding of other factors (proteins, other nucleic acids, etc.) needed for the proper function of the target i.e. operate by steric hindrance. For instance, the antisense oligonucleotide may bind to sequence motifs in either the pre-mRNA or mRNA that are important for recognition and binding of transacting factors involved in splicing, poly-adenylation, cellular transport, post-transcriptional modifications of nucleosides in the RNA, capping of the 5′-end, translation, etc. In the case of pre-mRNA splicing, the outcome of the interaction between the oligonucleotide and its target may be either suppression of expression of an undesired protein, generation of alternative spliced mRNA encoding a desired protein or both.

In another embodiment, binding of the oligonucleotide to its target disables the translation process by creating a physical block to the ribosomal machinery, i.e. tranlational arrest.

In yet another embodiment, binding of the oligonucleotide to its target interferes with the RNAs ability to adopt secondary and higher order structures that are important for its proper function, i.e. structural interference. For instance, the oligonucleotide may interfere with the formation of stem-loop structures that play crucial roles in different functions, such as providing additional stability to the RNA or adopting essential recognition motifs for different proteins.

In still another embodiment, binding of the oligonucleotide inactivates the target toward further cellular metabolic processes by recruiting cellular enzymes that degrades the mRNA. For instance, the oligonucleotide may comprise a segment of nucleosides that have the ability to recruit ribonuclease H (RNaseH) that degrades the RNA part of a DNA/RNA duplex. Likewise, the oligonucleotide may comprise a segment which recruits double stranded RNAses, such as for instance RNAseIII or it may comprise an external guide sequence (EGS) that recruit an endogenous enzyme (RNase P) which degrades the target mRNA Also, the oligonucleotide may comprise a sequence motif which exhibit RNAse catalytic activity or moieties may be attached to the oligonucleotides which when brought into proximity with the target by the hybridization event disables the target from further metabolic activities.

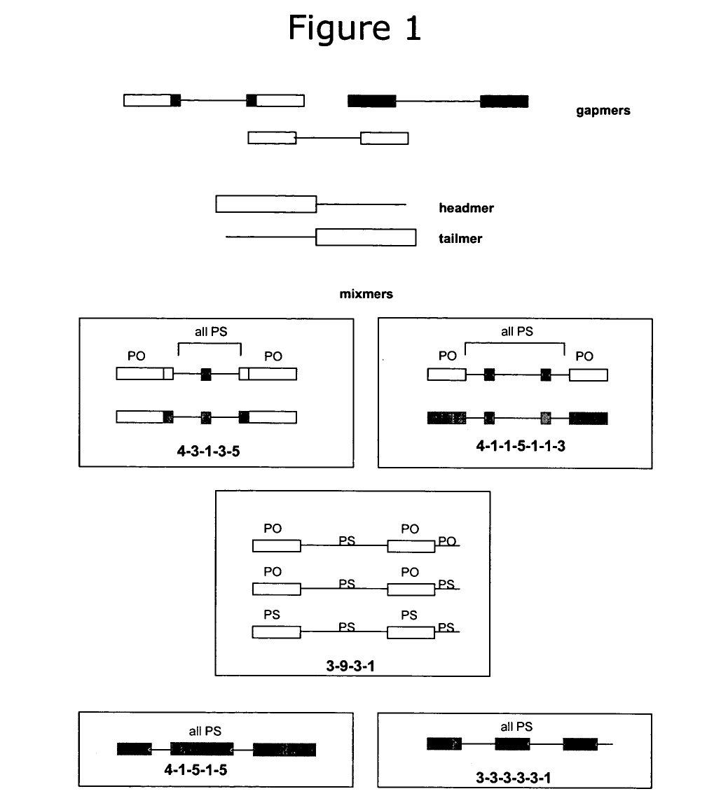

It has been shown that β-D-oxy-LNA does not support RNaseH activity. However, this can be changed according to the invention by creating chimeric oligonucleotides composed of β-D-oxy-LNA and DNA, called gapmers. A gapmer is based on a central stretch of 4-12 nt DNA or modified monomers recognizable and cleavable by the RNaseH (the gap) typically flanked by 1 to 6 residues of β-D-oxy-LNA (the flanks). The flanks can also be constructed with LNA derivatives. There are other chimeric constructs according to the invention that are able to act via an RNaseH mediated mechanism. A headmer is defined by a contiguous stretch of β-D-oxy-LNA or LNA derivatives at the 5′-end followed by a contiguous stretch of DNA or modified monomers recognizable and cleavable by the RNaseH towards the 3′-end, and a tailmer is defined by a contiguous stretch of DNA or modified monomers recognizable and cleavable by the RNaseH at the 5′-end followed by a contiguous stretch of β-D-oxy-LNA or LNA derivatives towards the 3′-end. Other chimeras according to the invention, called mixmers consisting of an alternate composition of DNA or modified monomers recognizable and cleavable by RNaseH and β-D-oxy-LNA and/or LNA derivatives might also be able to mediate RNaseH binding and cleavage. Since α-L-LNA recruits RNaseH activity to a certain extent, smaller gaps of DNA or modified monomers recognizable and cleavable by the RNaseH for the gapmer construct might be required, and more flexibility in the mixmer construction might be introduced. FIG. 1 shows an outline of different designs according to the invention.

The clinical effectiveness of antisense oligonucleotides depends to a significant extent on their pharmacokinetics e.g. absorption, distribution, cellular uptake, metabolism and excretion. In turn these parameters are guided significantly by the underlying chemistry and the size and three-dimensional structure of the oligonucleotide.

As mentioned earlier LNA according to the invention is not a single, but several related chemistries, which although molecularly different all exhibit stunning affinity towards complementary DNA and RNA, Thus, the LNA family of chemistries are uniquely suited of development oligos according to the invention with tailored pharmacokinetic properties exploiting either the high affinity of LNA to modulate the size of the active compounds or exploiting different LNA chemistries to modulate the exact molecular composition of the active compounds. In the latter case, the use of for instance amino-LNA rather than oxy-LNA will change the overall charge of the oligo and affect uptake and distribution behavior. Likewise the use of thio-LNA instead of oxy-LNA will increase the lipophilicity of the oligonucleotide and thus influence its ability to pass through lipophilic barriers such as for instance the cell membrane.

Modulating the pharmacokinetic properties of an LNA oligonucleotide according to the invention may further be achieved through attachment of a variety of different moieties. For instance, the ability of oligonucleotides to pass the cell membrane may be enhanced by attaching for instance lipid moieties such as a cholesterol moiety, a thioether, an aliphatic chain, a phospholipid or a polyamine to the oligonucleotide. Likewise, uptake of LNA oligonucleotides into cells may be enhanced by conjugating moieties to the oligonucleotide that interacts with molecules in the membrane, which mediates transport into the cytoplasm.

The pharmacodynamic properties can according to the invention be enhanced with groups that improve oligomer uptake, enhance biostability such as enhance oligomer resistance to degradation, and/or increase the specificity and affinity of oligonucleotides hybridisation characteristics with target sequence e.g. a mRNA sequence.

There are basically two types of toxicity associated with antisense oligos: sequence-dependant toxicity, involving the base sequence, and sequence-independent, class-related toxicity. With the exception of the issues related to immunostimulation by native CpG sequence motifs, the toxicities that have been the most prominent in the development of antisense oligonucleotides are independent of the sequence, e.g. related to the chemistry of the oligonucleotide and dose, mode, frequency and duration of administration. The phosphorothioates class of oligonucleotides have been particularly well characterized and found to elicit a number of adverse effects such as complement activation, prolonged PTT (partial thromboplastin time), thrombocytopenia, hepatotoxicity (elevation of liver enzymes), cardiotoxicity, splenomegaly and hyperplasia of reticuloendothelial cells.

As mentioned earlier, the LNA family of chemistries provide unprecedented affinity, very high bio-stablity and the ability to modulate the exact molecular composition of the oligonucleotide. In one embodiment of the invention, LNA containing compounds enables the development of oligonucleotides which combine high potency with little—if any—phosphorothioate linkages and which are therefore likely to display better efficacy and safety than contemporary antisense compounds.

Oligo- and polynucleotides of the invention may be produced using the polymerisation techniques of nucleic acid chemistry well known to a person of ordinary skill in the art of organic chemistry. Generally, standard oligomerisation cycles of the phosphoramidite approach (S. L. Beaucage and R. P. Iyer, Tetrahedron, 1993, 49, 6123; S. L. Beaucage and R. P. Iyer, Tetrahedron, 1992, 48, 2223) is used, but e.g. H-phosphonate chemistry, phosphortriester chemistry can also be used.

For some monomers of the invention longer coupling time, and/or repeated couplings with fresh reagents, and/or use of more concentrated coupling reagents were used.

The phosphoramidites employed coupled with satisfactory >95% step-wise coupling yields. Thiolation of the phosphate is performed by exchanging the normal, e.g. iodine/pyridine/H₂O, oxidation used for synthesis of phosphordiester oligomers with an oxidation using Beaucage's reagent (commercially available) other sulfurisation reagents are also comprised. The phosphorthioate LNA oligomers were efficiently synthesised with stepwise coupling yields >=98%.

The β-D-amino-LNA, β-D-thio-LNA oligonucleotides, α-L-LNA and β-D-methylamino-LNA oligonucleotides were also efficiently synthesised with step-wise coupling yields ≧98% using the phosphoramidite procedures.

Purification of LNA oligomeric compounds was done using disposable reversed phase purification cartridges and/or reversed phase HPLC and/or precipitation from ethanol or butanol. Capillary gel electrophoresis, reversed phase HPLC, MALDI-MS, and ESI-MS was used to verify the purity of the synthesized oligonucleotides. Furthermore, solid support materials having immobilised thereto an optionally nucleobase protected and optionally 5′-OH protected LNA are especially interesting as material for the synthesis of LNA containing oligomeric compounds where an LNA monomer is included in at the 3′ end. In this instance, the solid support material is preferable CPG, e.g. a readily (commercially) available CPG material or polystyrene onto which a 3′-functionalised, optionally nucleobase protected and optionally 5′-OH protected LNA is linked using the conditions stated by the supplier for that particular material.

Ha-ras is involved in a number of basic biological mechanisms including red blood cell proliferation, cellular proliferation, ion metabolism, glucose and energy metabolism, pH regulation and matrix metabolism. For example Ha-ras has been shown to be frequently mutated in bladder, thyroid, kidney carcinoma (Bos (1989), Cancer Research 49: 4682-4689). Over-expression of Ha-ras has been shown in breast and colon carcinoma (P. Horan Hand et al. (1987) Journal of the National Cancer Institute 79: 59-65) The methods of the invention is preferably employed for treatment or prophylaxis against diseases caused by cancer, particularly for treatment of cancer as may occur in tissue such as lung, breast, colon, prostate, pancreas, liver, brain, testes, stomach, intestine, bowel, spinal cord, sinuses, urinary tract or ovaries cancer.

The invention described herein encompasses a method of preventing or treating cancer comprising a therapeutically effective amount of a Ha-ras modulating oligomeric compound, including but not limited to high doses of the oligomer, to a human in need of such therapy. The invention further encompasses the use of a short period of administration of a Ha-ras modulating oligomeric compound. Normal, non-cancerous cells divide at a frequency characteristic for the particular cell type. When a cell has been transformed into a cancerous state, uncontrolled cell proliferation and reduced cell death results, and therefore, promiscuous cell division or cell growth is a hallmark of a cancerous cell type. Examples of types of cancer, include, but are not limited to, non-Hodgkin's lymphoma, Hodgkin's lymphoma, leukemia (e.g., acute leukemia such as acute lymphocytic leukemia, acute myelocytic leukemia, chronic myeloid leukemia, chronic lymphocytic leukemia, multiple myeloma), colon carcinoma, rectal carcinoma, pancreatic cancer, breast cancer, ovarian cancer, prostate cancer, renal cell carcinoma, hepatoma, bile duct carcinoma, choriocarcinoma, cervical cancer, testicular cancer, lung carcinoma, bladder carcinoma, melanoma, head and neck cancer, brain cancer, cancers of unknown primary site, neoplasms, cancers of the peripheral nervous system, cancers of the central nervous system, tumors (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, seminoma, embryonal carcinoma, Wilms' tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, and retinoblastoma), heavy chain disease, metastases, or any disease or disorder characterized by uncontrolled or abnormal cell growth.

It should be understood that the invention also relates to a pharmaceutical composition, which comprises a least one antisense oligonucleotide construct of the invention as an active ingredient. It should be understood that the pharmaceutical composition according to the invention optionally comprises a pharmaceutical carrier, and that the pharmaceutical composition optionally comprises further antisense compounds, chemotherapeutic compounds, anti-inflammatory compounds, antiviral compounds and/or immuno-modulating compounds.

The oligomeric compound comprised in this invention can be employed in a variety of pharmaceutically acceptable salts. As used herein, the term refers to salts that retain the desired biological activity of the herein identified compounds and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine; or (c) combinations of (a) and (b); e.g., a zinc tannate salt or the like.

In one embodiment of the invention the oligomeric compound may be in the form of a pro-drug. Oligonucleotides are by virtue negatively charged ions. Due to the lipophilic nature of cell membranes the cellular uptake of oligonucleotides are reduced compared to neutral or lipophilic equivalents. This polarity “hindrance” can be avoided by using the pro-drug approach (see e.g. Crooke, R. M. (1998) in Crooke, S. T. Antisense research and Application. Springer-Verlag, Berlin, Germany, vol. 131, pp. 103-140). In this approach the oligonucleotides are prepared in a protected manner so that the oligo is neutral when it is administered. These protection groups are designed in such a way that so they can be removed then the oligo is taken up be the cells. Examples of such protection groups are S-acetylthioethyl (SATE) or S-pivaloylthioethyl (t-butyl-SATE). These protection groups are nuclease resistant and are selectively removed intracellulary.

In one embodiment of the invention the oligomeric compound is linked to ligands/conjugates. It is way to increase the cellular uptake of antisense oligonucleotides. This conjugation can take place at the terminal positions 5′/3′-OH but the ligands may also take place at the sugars and/or the bases. In particular, the growth factor to which the antisense oligonucleotide may be conjugated, may comprise transferrin or folate. Transferrin-polylysine-oligonucleotide complexes or folate-polylysine-oligonucleotide complexes may be prepared for uptake by cells expressing high levels of transferrin or folate receptor. Other examples of conjugates/lingands are cholesterol moieties, duplex intercalators such as acridine, poly-L-lysine, “end-capping” with one or more nuclease-resistant linkage groups such as phosphoromonothioate, and the like.

The preparation of transferrin complexes as carriers of oligonucleotide uptake into cells is described by Wagner et al., Proc. Natl. Acad. Sci. USA 87, 3410-3414 (1990). Cellular delivery of folate-macromolecule conjugates via folate receptor endocytosis, including delivery of an antisense oligonucleotide, is described by Low et al., U.S. Pat. No. 5,108,921. Also see, Leamon et al., Proc. Natl. Acad. Sci. 88, 5572 (1991).

The invention also includes the formulation of one or more oligonucleotide compound as disclosed herein. Pharmaceutically acceptable binding agents and adjuvants may comprise part of the formulated drug. Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The oligonucleotide formulations may also be emulsions of the active pharmaceutical ingredients and a lipid forming a micellular emulsion.

An oligonucleotide of the invention may be mixed with any material that do not impair the desired action, or with material that supplement the desired action. These could include other drugs including other nucleoside compounds.

For parenteral, subcutaneous, intradermal or topical administration the formulation may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

Preferably, an oligomeric compound is included in a unit formulation such as in a pharmaceutically acceptable carrier or diluent in an amount sufficient to deliver to a patient a therapeutically effective amount without causing serious side effects in the treated patient.

The pharmaceutical compositions of the present invention may be administered in a number of ways depending upon whether local or systemic treatment is desired and upon the area to be treated. Administration may be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal or intraventricular, administration. In one embodiment the active oligo is administered IV, IP, orally, topically or as a bolus injection or administered directly in to the target organ.

Pharmaceutical compositions and formulations for topical administration may include transdermal patches, ointments, lotions, creams, gels, drops, sprays, suppositories, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Compositions and formulations for oral administration include but is not restricted to powders or granules, microparticulates, nanoparticulates, suspensions or solutions in water or non-aqueous media, capsules, gel capsules, sachets, tablets or minitablets. Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to tumour tissue may be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacol 2002; 54(1):3-27).

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels and suppositories. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Oligonucleotides of the invention may also be conjugated to active drug substances, for example, aspirin, ibuprofen, a sulfa drug, an antidiabetic, an antibacterial or an antibiotic.

LNA containing oligomeric compound are useful for a number of therapeutic applications as indicated above. In general, therapeutic methods of the invention include administration of a therapeutically effective amount of an LNA-modified oligonucleotide to a mammal, particularly a human.

In a certain embodiment, the present invention provides pharmaceutical compositions containing (a) one or more antisense compounds and (b) one or more other chemotherapeutic agents which function by a non-antisense mechanism. When used with the compounds of the invention, such chemotherapeutic agents may be used individually (e.g. mithramycin and oligonucleotide), sequentially (e.g. mithramycin and oligonucleotide for a period of time followed by another agent and oligonucleotide), or in combination with one or more other such chemotherapeutic agents or in combination with radiotherapy. All chemotherapeutic agents known to a person skilled in the art are here incorporated as combination treatments with compound according to the invention.

Anti-inflammatory drugs, including but not limited to nonsteroidal anti-inflammatory drugs and corticosteroids, antiviral drugs, and immuno-modulating drugs may also be combined in compositions of the invention. Two or more combined compounds may be used together or sequentially.

In another embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

The dosage is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient.

Optimum dosages may vary depending on the relative potency of individual oligonucleotides. Generally it can be estimated based on EC50s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

The LNA containing oligomeric compounds of the present invention can be utilized for as research reagents for diagnostics, therapeutics and prophylaxis . In research, the antisense oligonucleotides may be used to specifically inhibit the synthesis of ras genes in cells and experimental animals thereby facilitating functional analysis of the target or an appraisal of its usefulness as a target for therapeutic intervention. In diagnostics the antisense oligonucleotides may be used to detect and quantitate ras expression in cell and tissues by Northern blotting, in-situ hybridisation or similar techniques. For therapeutics, an animal or a human, suspected of having a disease or disorder, which can be treated by modulating the expression of ras is treated by administering antisense compounds in accordance with this invention. Further provided are methods of treating an animal particular mouse and rat and treating a human, suspected of having or being prone to a disease or condition, associated with expression of ras by administering a therapeutically or prophylactically effective amount of one or more of the antisense compounds or compositions of the invention.

### EXAMPLES

### Example 1

### Monomer Synthesis

The LNA monomer building blocks and derivatives thereof were prepared following published procedures and references cited therein, see:

WO 03/095467 A

D. S. Pedersen, C. Rosenbohm, T. Koch (2002) Preparation of LNA Phosphoramidites, Synthesis 6, 802-808.

M. D. Søensen, L. Kvaernø, T. Bryld, A. E. H\dot{a}kansson, B. Verbeure, G. Gaubert, P. Herdewijn, J. Wengel (2002) α-L-ribo-configured Locked Nucleic Acid (α-l-LNA): Synthesis and Properties, J. Am. Chem. Soc., 124, 2164-2176.

S. K. Singh, R. Kumar, J. Wengel (1998) Synthesis of Novel Bicyclo[2.2.1] Ribonucleosides: 2′-Amino- and 2′-Thio-LNA Monomeric Nucleosides, J. Org. Chem. 1998, 63, 6078-6079.

C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663.

Synthesis of the 2′-thio-LNA ribothymidine phosphoramidite. Reagents and conditions: i) Pd/C, H₂, acetone, MeOH; ii) BzCl, pyridine, DMF; iii) 0.25 M H₂SO₄ (aq), DMF, 80° C. (79% from 4; 3 steps); iv) Tf₂O, DMAP, CH₂Cl₂, 0° C.; v) Na₂S, DMF (72% from 7; 2 steps); vi) NaOBz, DMF, 100° C. (81%); vii) NH₃, MeOH (76%); viii) DMT-Cl, pyridine (88%); ix) P(OCH₂CH₂CN)(N(ⁱPr)₂)₂, 4,5-dicyanoimidazole, CH₂Cl₂ (99%). DMT=4,4′-dimethoxytrityl, PN₂=2-cyanoethoxy(diisopropylamino)phosphinoyl.

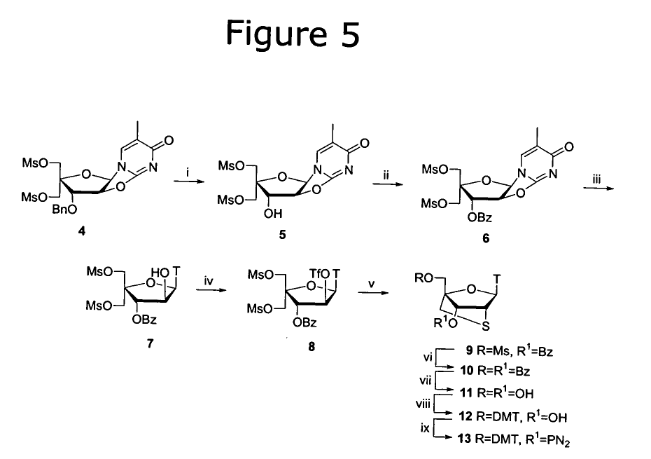

### 1-(3-O-Benzoyl-5-O-methanesulfonyl-4-C-methanesulfonyloxymethyl-β-D-threo-pentofuranosyl)thymine (7, FIG. 5)

Anhydro-nucleoside 4 (C. Rosenbohm, S. M. Christensen, M. D. Sørensen, D. S. Pedersen, L. E. Larsen, J. Wengel, T. Koch (2003) Synthesis of 2′-amino-LNA: a new strategy, Org. Biomol. Chem. 1, 655-663) (30.0 g, 58.1 mmol) was heated to 70° C. in a mixture of methanol (1000 cm³) and acetone (1000 cm³) until a clear solution was obtained and the solution was allowed to reach room temperature. The reaction flask was flushed with argon and Pd/C (10 wt. % Pd on carbon, 6.2 g, 5.8 mmol) was added. The mixture was stirred vigorously under an atmosphere of hydrogen gas (balloon). After 23 h the slurry was filtered through a pad of celite. The catalyst was recovered from the celite and refluxed in DMF (1000 cm³) for 1 h. The hot DMF slurry was filtered through a pad of celite and the organic layers combined and evaporated in vacuo to give nucleoside 5 as a yellow powder. Residual solvents were removed on a high vacuum pump overnight.

The crude nucleoside 5 (23 g) was heated to 70° C. in DMF (300 cm³) to give a clear yellow solution that was allowed to cool to room temperature. Benzoyl chloride (81.7 g, 581 mmol, 67.4 cm³) was added followed by pyridine (70 cm³). After 18 h the reaction was quenched with methanol (200 cm³) and excess methanol was removed in vacuo.

To the dark brown solution of nucleoside 6 aqueous H₂SO₄ (0.25 M, 400 cm³) was added. The solution was heated to 80° C. on an oil bath (At approx 50° C. precipitation occurs. The solution becomes clear again at 80° C.). After 22 h at 80° C. the solution was allowed to cool to room temperature. The reaction mixture was transferred to a separatory funnel with ethyl acetate (1000 cm³). The organic layer was washed with sat. aq NaHCO₃ (2×1000 cm³). The combined aqueous layers were extracted with ethyl acetate (1000+500 cm³). The organic layers were combined and washed with sat. aq NaHCO₃ (1000 cm³), dried (Na₂SO₄), filtered and evaporated in vacuo to give a yellow liquid. Residual solvents were removed on a high vacuum pump overnight to give a yellow syrup. The product was purified by Dry Column Vacuum Chromatography (id 10 cm; 100 cm³ fractions; 50-100% EtOAc in n-heptane (v/v)—10% increments; 2-24% MeOH in EtOAc (v/v)—2% increments). Fractions containing the product were combined and evaporated in vacuo giving nucleoside 7 (25.1 g, 79%) as a white foam.

R_{f}=0.54 (5% MeOH in EtOAc, v/v); ESI-MS m/z found 549.0 ([MH]⁺, calcd 549.1); ¹H NMR (DMSO-d₆) δ 11.39 (br s, 1H, NH), 8.10-8.08 (m, 2H, Ph), 7.74-7.70 (m, 1H, Ph), 7.60-7.56 (m, 2H, Ph), 7.51 (d, J=1.1 Hz, 1H, H6), 6.35 (d, J=4.9 Hz, 1H, h1′), 6.32 (d, J=5.3 Hz, 1H, 2′-OH), 5.61 (d, J=4.0 Hz, 1H, H3′), 4.69 (d, J=10.8 Hz, 1H), 4.59 (m, 1H, H2′), 4.55 (d, J=10.8 Hz, 1H), 4.52 (d, J=10.8 Hz, 1H), 4.46 (d, J=10.6 Hz, 1H) (H5′ and H1″), 3.28 (s, 3H, Ms), 3.23 (s, 3H, Ms), 1.81 (s, 3H, CH₃); ¹³C NMR (DMSO-d₆) δ 164.5, 163.6 (C4, PhC(O)), 150.3 (C2), 137.7 (C6), 133.8, 129.6, 128.7, 128.6 (Ph), 108.1 (C5), 84.8 (C1′), 81.1 (C4′), 78.0 (C3′), 73.2 (C2′), 68.0, 67.1 (C5′, C1″), 36.7, 36.6 (2×Ms), 11.9 (CH₃); Elemental anal. calcd for C₂₀H₂₄N₂O₁₂S₂. 0.33 H₂O (%): C, 44.34; H, 4.65; N, 4.85. Found: C, 44.32; H, 4.58; N, 4.77.

### (1R,3R,4R,7R)-7-Benzoyloxy-1-methansulfonyloxymethyl-3-(thymin-1-yl)-2-oxa-5-thiabicyclo[2:2:1]heptane (9)

1-(3-O-Benzoyl-5-O-methanesulfonyl-4-C-methanesulfonyloxymethyl-β-D-threopentofuranosyl)thymine (7) (10.00 g, 18.23 mmol) was dissolved in dichloromethane (500 cm³) and cooled to 0° C. Pyridine (15 cm³) and DMAP (8.91 g, 72.9 mmol) was added followed by dropwise addition of trifluoromethanesulfonic anhydride (10.30 g, 36.5 mmol, 6.0 cm³). After 1 h the reaction was quenched with sat. aq NaHCO₃ (500 cm³) and transferred to a separatory funnel. The organic layer was washed with 1.0 M aq HCl (500 cm³), sat. aq NaHCO₃ (500 cm³) and brine (500 cm³). The organic layer was evaporated in vacuo with toluene (100 cm³) to give 1-(3-O-benzoyl-5-O-methanesulfonyl-4-C-methanesulfonyloxymethyl-2-O-trifluoromethanesulfonyl-β-D-threopentofuranosyl)thymine (8) as a yellow powder.

The crude nucleoside 8 was dissolved in DMF (250 cm³) and Na₂S (1.57 g, 20.1 mmol) was added to give a dark green slurry. After 3 h the reaction was quenched with half sat. aq NaHCO₃ (500 cm³) and extracted with dichloromethane (500+2×250 cm³). The combined organic layers were washed with brine (500 cm³), dried (Na₂SO₄), filtered and concentrated in vacuo to give a yellow liquid. Residual solvent was removed overnight on a high vacuum pump to give a yellow gum that was purified by Dry Column Vacuum Chromatography (id 6 cm: 50 cm³ fractions; 50-100% EtOAc in n-heptane (v/v)—10% increments; 2-20% MeOH in EtOAc (v/v)—2% increments) to give nucleoside 9 (6.15 g, 72%) as a yellow foam.

R_{f}=0.27 (20% n-heptane in EtOAc, v/v); ESI-MS m/z found 469.0 ([MH]⁺, calcd 469.1); ¹H NMR (CDCl₃) δ 8.70 (br s, 1H, NH), 8.01-7.99 (m, 2H, Ph), 7.67 (d, J=1.1 Hz, 1H, H6), 7.65-7.61 (m, 1H, Ph), 7.50-7.46 (m, 2H, Ph), 5.98 (s, 1H, H1′), 5.34 (d, J=2.4 Hz, 1H, H3′), 4.66 (d, J=11.7 Hz, 1H, H5′a), 4.53 (d, J=11.5 Hz, 1H, H5′b), 4.12 (m (overlapping with residual EtOAc), 1H, H2′), 3.15-3.13 (m, 4H, H1″a and Ms), 3.06 (d, J=10.6 Hz, 1H, H1−b), 1.98 (d, J=1.1 Hz, 3H, CH₃); ¹³C NMR (CDCl₃) δ 165.2, 163.5 (C4, PhC(O)), 149.9 (C2), 134.1, 133.9, 129.8, 128.7, 128.3 (C6, Ph), 110.7 (C5), 91.1 (C1′), 86.8 (C4′), 72.6 (C3′), 65.8 (C5′), 50.5 (C2′), 37.9 (Ms), 35.1 (Cl″), 12.5 (CH₃); Elemental anal. calcd for C₁₉H₂₀N₂O₈S₂.0.33 EtOAc (%): C, 49.21; H, 4.72; N, 5.47. Found: C, 49.25; H, 4.64; N, 5.48.

### (1R,3R,4R,7R)-7-Benzoyloxy-1-benzoyloxymethyl-3-(thymin-1-yl)-2-oxa-5-thiabicyclo[2:2:1]heptane (10)

Nucleoside 9 (1.92 g, 4.1 mmol) was dissolved in DMF (110 cm³). Sodium benzoate (1.2 g, 8.2 mmol) was added and the mixture was heated to 100° C. for 24 h. The reaction mixture was transferred to a separatory funnel with half sat. brine (200 cm³) and extracted with ethyl acetate (3×100 cm³). The combined organic layers were dried (Na₂SO₄), filtered and evaporated in vacuo to give a brown liquid. The product was put on a high vacuum pump to remove residual solvent. The resulting brown gum was purified by Dry Column Vacuum Chromatography (id 4 cm; 50 cm³ fractions; 0-100% EtOAc in n-heptane (v/v)—10% increments; 2-10% MeOH in EtOAc (v/v)—2% increments) to give nucleoside 10 (1.64 g, 81%) as a slightly yellow foam.

R_{f}=0.57 (20% n-heptane in EtOAc, v/v); ESI-MS m/z found 495.1 ([MH]⁺, calcd 495.1); ¹H NMR (CDCl₃) δ 9.02 (br s, 1H, NH), 8.07-7.99 (m, 4H, Ph), 7.62-7.58 (m, 2H, Ph), 7.47-7.42 (m, 5H, Ph and H6), 5.95 (s, 1H, H1′), 5.46 (d, J=2.2 Hz, 1H, H3′), 4.93 (d, J=12.8 Hz, 1H, H5′a), 4.60 (d, J=12.8 Hz, 1H, H5′b), 4.17 (d, J=2.2 Hz, 1H, H2′), 3.27 (d, J=10.6 Hz, 1H, H1″a), 3.16 (d, J=10.6 Hz, 1H, H1″b), 1.55 (d, J=1.1 Hz, 3H, CH₃);

¹³C NMR (CDCl₃) δ 165.8, 165.1, 163.7 (C4, 2×PhC(O)), 150.0 (C2), 133.9, 133.7, 133.6, 129.8, 129.6, 129.0, 128.8, 128.6, 128.5 (C6, 2×Ph), 110.3 (C5), 91.3 (C1′), 87.5 (C4′), 72.9 (C3′), 61.3 (C5′), 50.6 (C2′), 35.6 (C1″), 12.3 (CH₃);

Elemental anal. calcd for C₂₅H₂₂N₂O₇S (%): C, 60.72; H, 4.48; N, 5.66. Found: C, 60.34; H, 4.49; N, 5.35.

### (1R,3R,4R,7R)-7-Hydroxy-1-hydroxymethyl-3-(thymin-1-yl)-2-oxa-5-thiabicyclo[2:2:1]heptane (11)

Nucleoside 10 (1.50 g, 3.0 mmol) was dissolved in methanol saturated with ammonia (50 cm³). The reaction flask was sealed and stirred at ambient temperature for 20 h. The reaction mixture was concentrated in vacuo to give a yellow gum that was purified by Dry Column Vacuum Chromatography (id 4 cm; 50 cm³ fractions; 0-16% MeOH in EtOAc (v/v)—1% increments) giving nucleoside 11 (0.65 g, 76%) as clear needles.

R_{f}=0.31 (10% MeOH in EtOAc, v/v); ESI-MS m/z found 287.1 ([MH]⁺, calcd 287.1); ¹H NMR (DMSO-d₆) δ 11.32 (br s, 1H, NH), 7.96 (d, J=1.1 Hz, 1H, H6), 5.95 (s, 1H, H6), 5.70 (d, J=4.2 Hz, 1H, 3′-OH), 5.62 (s, 1H, H1′), 4.49 (t, J=5.3 Hz, 1H, 5′-OH), 4.20 (dd, J=4.1 and 2.1 Hz, 1H, H3′), 3.77-3.67 (m, 2H, H5═), 3.42 (d, J=2.0 H, 1H, H2′), 2.83 (d, J=10.1 Hz, 1H, H1″a), 2.64 (d, J=10.1 Hz, 1H, H1″b), 1.75 (d, J=1.1 Hz, 3H, CH₃); ¹³C NMR (DMSO-d₆) δ 163.8 (C4), 150.0 (C2), 135.3 (C6), 107.5 (C5), 90.2, 89.6 (1′ and C4′), 69.4 (C3′), 58.0 (C5′), 52.1 (C2′), 34.6 (C1″), 12.4 (CH₃); Elemental anal. calcd for C₁₁H₁₄N₂O₅S (%): C, 46.15; H, 4.93; N, 9.78. Found: C, 46.35; H, 4.91; N, 9.54.

### (1R,3R,4R,7R)-1-(4,4′-Dimethoxytrityloxymethyl )-7-hydroxy-5-methyl-3-(thymin-1-yl)-2-oxa-5-thiabicyclo[2:2:1]heptane (12)

Nucleoside 11 (0.60 g, 2.1 mmol) was dissolved in pyridine (10 cm³). 4,4′-Dimethoxytrityl chloride (0.88 g, 2.6 mmol) was added and the reaction was stirred at ambient temperature for 3 h. The reaction mixture was transferred to a separatory funnel with water (100 cm³) and extracted with ethyl acetate (100+2×50 cm³). The combined organic layers were washed with sat. aq NaHCO₃ (100 cm³), brine (100 cm³) and evaporated to dryness in vacuo to give a viscous yellow liquid. The product was redissolved in toluene (50 cm³) and concentrated in vacuo to give a yellow foam. The foam was dried on a high vacuum pump overnight and purified by Dry Column Vacuum Chromatography (id 4 cm; 50 cm³ fractions; 10-100% EtOAc in n-heptane (v/v)—10% increments) giving nucleoside 12 (1.08 g, 88%) as a white foam.

R_{f}=0.24 (20% n-heptane in EtOAc, v/v);

ESI-MS m/z found 587.1 ([M−H]⁺, calcd 587.2); ¹H NMR (CDCl₃) δ8.96 (br s, 1H, NH), 7.74 (d, J=1.1 Hz, 1H, H6), 7.46-7.44 (m, 2H, Ph), 7.35-7.22 (m, 9H, Ph), 7.19-7.15 (m, 2H, Ph), 6.86-6.80 (m, 2H, Ph), 5.82 (s, 1H, H1′), 4.55 (dd, J=9.3 and 2.1 Hz, 1H, H3′), 3.79 (s, 6H, OCH₃), 3.71 (d, J=2.0 Hz, 1H, H2′), 3.50 (s, 2H, H5′), 2.81 (d, J=10.8 Hz, 1H, H1″a), 2.77 (d, J=10.8 Hz, 1H, H1″b), 2.69 (d, J=9.2 Hz, 1H, 3′-OH), 1.42 (s, 3H, CH₃);

¹³C NMR (CDCl₃) δ 158.7 (C4), 150.1 (C2), 144.1, 135.2, 135.1, 130.1, 129.1, 128.1, 128.0, 127.1, 127.0, 113.3 (C6, 3×Ph), 110.0 (C5), 90.2 (C(Ph)₃), 89.6 (C1′), 87.0 (C4′), 71.7 (C3′), 60.9 (C5′), 55.2 (C2′), 34.7 (C1″), 12.2 (CH₃); Elemental anal. calcd for C₃₂H₃₂N₂O₇S.0.5 H₂O (%): C, 64.31; H, 5.57; N, 4.69. Found: C, 64.22; H, 5.67; N, 4.47.

### (1R,3R,4R,7R)-7-(2-Cyanoethoxy(diisopropylamino)phosphinoxy)-1-(4,4′-dimethoxytrityloxymethyl)-3-(thymin-1-yl)-2-oxa-5-thiabicyclo[2.2.1]heptane (13)

According to the published method (D. S. Pedersen, C. Rosenbohm, T. Koch (2002) Preparation of LNA Phosphoramidites, Synthesis, 6, 802-808) nucleoside 12 (0.78 g, 1.33 mmol) was dissolved in dichloromethane (5 cm³) and a 1.0 M solution of 4,5-dicyanoimidazole in acetonitrile (0.93 cm³, 0.93 mmol) was added followed by dropwise addition of 2-cyanoethyl-N,N,N′N′-tetraisopropylphosphorodiamidite (0.44 cm³, 1.33 mmol). After 2 h the reaction was transferred to a separatory funnel with dichloromethane (40 cm³) and washed with sat. aq NaHCO₃ (2×25 cm³) and brine (25 cm³). The organic layer was dried (Na₂SO₄), filtered and evaporated in vacuo to give nucleoside 13 (1.04 g, 99%) as a white foam. R_{f}=0.29 and 0.37—two diastereoisomers (20% n-heptane in EtOAc, v/v); ESI-MS m/z found 789.3 ([MH]⁺, calcd 789.3); ³¹P NMR (DMSO-d₆) δ 150.39, 150.26.

### Example 2

### Oligonucleotide Synthesis

Oligonucleotides were synthesized using the phosphoramidite approach on an Expedite 8900/MOSS synthesizer (Multiple Oligonucleotide Synthesis System) at 1 or at 15 μmol. At the end of the synthesis (DMT-on) the oligonucleotides were cleaved from the solid support using aqueous ammonia for 1 h at room temperature, and further deprotected for 3 h at 65° C. The oligonucleotides were purified by reverse phase HPLC (RP-HPLC). After the removal of the DMT-group, the oligonucleotides were characterized by IE-HPLC or RP-HPLC. The identity of the oligonucleotides is confirmed by ESI-MS. See below for more details.

Preparation of the LNA Succinyl Hemiester

5′-O-Dmt-3′-hydroxy-LNA monomer (500 mg), succinic anhydride (1.2 eq.) and DMAP (1.2 eq.) were dissolved in DCM (35 mL). The reaction was stirred at room temperature overnight. After extractions with NaH₂PO₄ 0.1 M pH 5.5 (2×) and brine (1×), the organic layer was further dried with anhydrous Na₂SO₄ filtered and evaporated. The hemiester derivative was obtained in 95% yield and was used without any further purification.

Preparation of the LNA-support

The above prepared hemiester derivative (90 μmol) was dissolved in a minimum amount of DMF, DIEA and pyBOP (90 μmol) were added and mixed together for 1 min. This preactivated mixture was combined with LCM-CPG (500 Å, 80-120 mesh size, 300 mg) in a manual synthesizer and stirred. After 1.5 h at room temperature, the support was filtered off and washed with DMF, DCM and MeOH. After drying the loading was determined to be 57 μmol/g (see Tom Brown, Dorcas J. S. Brown, “Modern machine-aided methods of oligodeoxyribonucleotide synthesis”, in: F. Eckstein, editor. Oligonucleotides and Analogues A Practical Approach. Oxford: IRL Press, 1991: 13-14).

Elongation of the Oligonucleotide

The coupling of phosphoramidites (A(bz), G(ibu), 5-methyl-C(bz)) or T-β-cyanoethyl-phosphoramidite) is performed by using a solution of 0.1 M of the 5′-O-DMT-protected amidite in acetonitrile and DCI (4,5-dicyanoimidazole) in acetonitrile (0.25 M) as activator. The thiolation is carried out by using xanthane chloride (0.01 M in acetonitrile:pyridine 10%). The rest of the reagents are the ones typically used for oligonucleotide synthesis. Purification by RP-HPLC:

| | |
|---|---|
| Column: | XTerra, RP18, 5 μm, 7.8 × 50 mm column. |
| Eluent: | Eluent A: 0.1 M NH₄OAc, pH: 10. |
| | Eluent B: Acetonitrile |
| Flow: | 5 ml/min. |

Gradient:

| Time (min.) | Eluent A | Eluent B |
|---|---|---|
| 0.05 min. | 95% | 5% |
| 5 min. | 95% | 5% |
| 12 min. | 65% | 35% |
| 16 min. | 0% | 100% |
| 19 min. | 0% | 100% |
| 21 min | 100% | 0% |

Analysis by IE-HPLC:

| | |
|---|---|
| Column: | Dionex, DNAPac PA-100, 2 × 250 mm column. |
| Eluent: | Eluent A: 20 mM Tris-HCl, pH 7.6; |
| | 1 mM EDTA; 10 mM NaClO₄. |
| | Eluent B: 20 mM Tris-HCl, pH 7.6; |
| | 1 mM EDTA; 1 M NaClO₄. |
| Flow: | 0.25 ml/min. |

Gradient:

| Time (min.) | Eluent A | Eluent B |
|---|---|---|
| 1 min. | 95% | 5% |
| 10 min. | 65% | 35% |
| 11 min. | 0% | 100% |
| 15 min. | 0% | 100% |
| 16 min | 95% | 5% |
| 21 min. | 95% | 5% |

Abbreviations

DMT: Dimethoxytrityl

DCI: 4,5-Dicyanoimidazole

DMAP: 4-Dimethylaminopyridine

DCM: Dichloromethane

DMF: Dimethylformamide

THF: Tetrahydrofurane

DIEA: N,N-diisopropylethylamine

PyBOP: Benzotriazole-1-yl-oxy-tris-pyrrolidino-phosphonium hexafluorophosphate

Bz: Benzoyl

Ibu: Isobutyryl

### Example 3

### Test of Design of the Oligomeric Compound

It was of our interest to evaluate the antisense activity of oligonucleotides with different designs, in order to prove the importance of choosing the best design for an oligonucleotide targeting Ha-Ras. For this purpose, we set up an in vitro assay that would allow us to screen many different oligonucleotide designs by measuring the activity of the firefly (Photinus pyralis) luciferase after down-regulation by antisense oligonucleotides. FIG. 1 contains an illustration of the designs mentioned in the text.

In a first screen, designs containing β-D-oxy-LNA, which were all targeting the same motif within the mRNA were evaluated. Designs consisting of gapmers with a different gap-size, a different load of phosphorothioate internucleoside linkages, and a different load of LNA were tested. Headmers and tailmers with a different load of β-D-oxy-LNA, a different load of phosphorothioate internucleoside linkages and a different load of DNA were prepared. Mixmers of various compositions, which means that bear an alternate number of units of β-D-oxy-LNA, α-L-LNA and DNA, were also analysed in the in vitro assay. Moreover, LNA derivatives were also included in different designs, and their antisense activity was assessed. The importance of a good design is reflected by the data that can be obtained in a luciferase assay. The luciferase expression levels are measured in %, and give an indication of the antisense activity of the different designs containing β-D-oxy-LNA and LNA derivatives. We can easily see that some designs are potent antisense oligonucleotides, while others give moderate to low down-regulation levels. Therefore, a close correlation between good antisense activity and optimal design of an oligonucleotide is very evident. We appreciated good levels of down-regulation with various designs. Gapmers with gaps of 7-10 nt DNA and thiolation all over the backbone or with thiolation exclusively in the gap and PO in the flanks showed good results. These designs contain β-D-oxy-LNA or LNA derivatives. Headmers of 6 nt and 8 nt β-D-oxy-LNA also presented good levels of down-regulation, when the phosphorothioate internucleoside linkages are all over the backbone or only in the DNA-segment. Different mixmers gave good antisense activity in the luciferase assay. The alternate number of units of each α-L-oxy-LNA, β-D-oxy-LNA or DNA composition defines the mixmers, see FIG. 1. A mixmer 3-9-3-1, which has a deoxynucleoside residue at the 3′-end showed significant levels of down-regulation. In a mixmer 4-1-1-5-1-1-3, we placed two α-L-oxy-LNA residues interrupting the gap, being the flanks β-D-oxy-LNA. Furthermore, we interrupted the gap with two α-L-oxy-LNA residues, and substituted both flanks with α-L-oxy-LNA. Both designs presented significant levels of down-regulation. The presence of α-L-oxy-LNA might introduce a flexible transition between the North-locked flanks (oxy-LNA) and the α-L-oxy-LNA residue by spiking in deoxynucleotide residues. It is also interesting to study design 4-3-1-3-5 where a α-L-oxy-LNA residue interrupts the DNA stretch. In addition to the α-L-oxy-LNA in the gap, we also substituted two oxy-LNA residues at the edges of the flanks with two α-L-oxy-LNA residues. The presence of just one β-D-oxy-LNA residue (design 4-3-1-3-5) interrupting the stretch of DNAs in the gap results in a dramatic loss of down-regulation. Just by using α-L-oxy-LNA instead, the design shows significant down-regulation at 50 nM oligonucleotide concentration. The placement of α-L-oxy-LNA in the junctions and one α-L-oxy-LNA in the middle of the gap also showed down-regulation. α-L-oxy-LNA reveals to be a potent tool enabling the construction of different mixmers, which are able to present high levels of antisense activity. Other mixmers such as 4-1-5-1-5 and 3-3-3-3-3-1 can also be prepared. We can easily see that some designs are potent antisense oligonucleotides, while others give moderate to low down-regulation levels. Therefore, again a close correlation between good antisense activity and optimal design of an oligonucleotide is very evident. Other preferred designs are (1-3-8-3-1) where DNA residues are located in the flanks with 3 β-D-oxy-LNA monomers at each side of the gap. A further preferred design is (4-9-3-1) with D-oxy-LNA flanks and a 9 gap with a DNA at the 3′-end.

Assay

X1/5 Hela cell line (ECACC Ref. No: 95051229), which was stably transfected with a “tet-off” luciferase system, was used. In the absence of tetracycline the luciferase gene is expressed constitutively. The expression can be measured as light in a luminometer, when the luciferase substrate, luciferin is added. The X1/5 Hela cell line was grown in Minimun Essential Medium Eagle (Sigma M2279) supplemented with 1× Non Essential Amino Acid (Sigma M7145), 1× Glutamax I (Invitrogen 35050-038), 10% FBS calf serum, 25 μg/ml Gentamicin (Sigma G1397), 500 μg/ml G418 (Invitrogen 10131-027) and 300 μg/ml Hygromycin B (Invitrogen 10687-010). The X1/5 Hela cells were seeded at a density of 8000 cells per well in a white 96 well plate (Nunc 136101) the day before the transfection. Before the transfection, the cells were washed one time with OptiMEM (Invitrogen) followed by addition of 40 μl OptiMEM with 2 μg/ml of Lipofectamine2000 (Invitrogen). The cells were incubated for 7 minutes before addition of the oligonucleotides. 10 μl of oligonucleotide solutions were added and the cells were incubated for 4 h at 37° C. and 5% CO₂. After the 4 h incubation, the cells were washed once in OptiMEM and growth medium was added (100 μl). The luciferase expression was measure the next day. Luciferase expression was measured with the Steady-Glo luciferase assay system from Promega. 100 μl of the Steady-Glo reagent was added to each well and the plate was shaken for 30 s at 700 rpm. The plate was read in Luminoskan Ascent instrument from ThermoLabsystems after 8 min of incubation to complete total lysis of the cells. The luciferase expression is measured as Relative Light Units per seconds (RLU/s). The data was processed in the Ascent software (v2.6) and graphs were drawn in SigmaPlot2001.

### Example 4

### In Vitro Model: Cell Culture

The effect of antisense compounds on target nucleic acid expression can be tested in any of a variety of cell types provided that the target nucleic acid is present at measurable levels. Target can be expressed endogenously or by transient or stable transfection of a nucleic acid encoding said nucleic acid. The expression level of target nucleic acid can be routinely determined using, for example, Northern blot analysis, Real-Time PCR, Ribonuclease protection assays. The following cell types are provided for illustrative purposes, but other cell types can be routinely used, provided that the target is expressed in the cell type chosen.

Cells were cultured in the appropriate medium as described below and maintained at 37° C. at 95-98% humidity and 5% CO₂. Cells were routinely passaged 2-3 times weekly.

15PC3: The human prostate cancer cell line 15PC3 was kindly donated by Dr. F. Baas, Neurozintuigen Laboratory, AMC, The Netherlands and was cultured in DMEM (Sigma)+10% fetal bovine serum (FBS)+Glutamax I+gentamicin

A549: The human non-small cell lung cancer cell line A549 was purchased from ATCC, Manassas and was cultured in DMEM (Sigma)+10% FBS+Glutamax I+gentamicin

MCF7: The human breast cancer cell line MCF7 was purchased from ATCC and was cultured in Eagle MEM (Sigma)+10% FBS+Glutamax I+gentamicin

SW480: The human colon cancer cell line SW480 was purchased from ATCC and was cultured in L-15 Leibovitz (Sigma)+10% FBS+Glutamax I+gentamicin

SW620: The human colon cancer cell line SW620 was purchased from ATCC and was cultured in L-15 Leibovitz (Sigma)+10% FBS+Glutamax I+gentamicin

HT29: The human prostate cancer cell line HT29 was purchased from ATCC and was cultured in McCoy's 5a MM (Sigma)+10% FBS+Glutamax I+gentamicin

NCI H23: The human non-small-cell lung cancer cell line was purchased from ATCC and was cultured in RPMI 1640 with Glutamax I (Gibco)+10% FBS+HEPES+gentamicin

HCT-116: The human colon cancer cell line HCT-116 was purchased from ATCC and was cultured in McCoy's 5a MM+10% FBS+Glutamax I+gentamicin

MDA-MB-231: The human breast cancer cell line MDA-MB-231 was purchased from ATCC and was cultured in L-15 Leibovitz+10% FBS+Glutamax I+gentamicin

MDA-MB-435s: The human breast cancer cell line MDA-MB-435s was purchased from ATCC and was cultured in L-15 Leibovitz+10% FBS+Glutamax I+gentamicin

DMS273: The human small-cell lung cancer cell line DMS273 was purchased from ATCC and was cultured in Waymouth with glutamine (Gibco)+10% FBS+gentamicin

PC3: The human prostate cancer cell line PC3 was purchased from ATCC and was cultured in F12 Coon's with glutamine (Gibco)+10% FBS+gentamicin

U373: The human glioblastoma astrocytoma cancer cell line U373 was purchased from ECACC and was cultured in EMEM+10% FBS+glutamax+NEAA+sodiumpyrovate+gentamicin.

### HUVEC-C Human Umbilical Vein End Thelial Cells were Purchased from ATCC and Propagated According to the Manufacturers Instructions

HMVEC-d (DMVEC's—dermal human microvascular endothelial cells) were purchased from Clonetics and cultured as described by manufacturer.

HMVEC human microvascular endothelial cells were purchased from Clonetics and cultured as stated by manufacturer

Human embryonic lung fibroblasts were purchased from ATCC and cultured as described by manufacturer

HMEC-1 Human mammary epithelial cells were purchased from Clonetics and maintained as recommended by the manufacturer

### Example 5

### In Vitro Model: Treatment with Antisense Oligonucleotide

The cells were treated with oligonucleotide using the cationic liposome formulation LipofectAMINE 2000 (Gibco) as transfection vehicle. Cells were seeded in 12-well cell culture plates (NUNC) and treated when 80-90% confluent. Oligo concentrations used ranged from 125 nM to 0,2 nM final concentration. Formulation of oligo-lipid complexes were carried out essentially as described in Dean et al. (Journal of Biological Chemistry 1994, 269, 16416-16424) using serum-free OptiMEM (Gibco) and a final lipid concentration of 10 μg/ml LipofectAMINE 2000 in 500 μl total volume. Cells were incubated at 37° C. for 4 hours and treatment was stopped by removal of oligo-containing culture medium. Cells were washed and serum-containing media was added. After oligo treatment cells were allowed to recover for 18 hours before they were harvested for RNA or protein analysis.

### Example 6

### In Vitro Model: Extraction of RNA and cDNA Synthesis

Total RNA Isolation

Total RNA was isolated either using RNeasy mini kit (Qiagen cat. no. 74104) or using the Trizol reagent (Life technologies cat. no. 15596). For RNA isolation from cell lines, RNeasy is the preferred method and for tissue samples Trizol is the preferred method. Total RNA was isolated from cell lines using the Qiagen RNA OPF Robot—BIO Robot 3000 according to the protocol provided by the manufacturer. Tissue samples were homogenised using an Ultra Turrax T8 homogeniser (IKA Analysen technik) and total RNA was isolated using the Trizol reagent protocol provided by the manufacturer.

First Strand Synthesis

First strand synthesis was performed using OmniScript Reverse Transcriptase kit (cat# 205113, Qiagen) according to the manufacturers instructions.

For each sample 0.5 μg total RNA was adjusted to 12 μl each with RNase free H₂O and mixed with 2 μl poly (dT)₁₂₋₁₈ (2.5 μg/ml) (Life Technologies, GibcoBRL, Roskilde, DK), 2 μl dNTP mix (5 mM each dNTP), 2 μl 10× Buffer RT, 1 μl RNAguard™Rnase INHIBITOR (33.3 U/ml), (cat# 27-0816-01, Amersham Pharmacia Biotech, Hørsholm, DK) and 1 μl OmniScript Reverse Transcriptase (4 U/μl) followed by incubation at 37° C. for 60 minutes and heat inactivation of the enzyme at 93° C. for 5 minutes.

### Example 7

### In Vitro Model: Analysis of Oligonucleotide Inhibition of Ha-Ras Expression by Real-Time PCR

Antisense modulation of Ha-ras expression can be assayed in a variety of ways known in the art. For example, Ha-ras mRNA levels can be quantitated by, e.g., Northern blot analysis, competitive polymerase chain reaction (PCR), or real-time PCR. Real-time quantitative PCR is presently preferred. RNA analysis can be performed on total cellular RNA or mRNA.

Methods of RNA isolation and RNA analysis such as Northern blot analysis is routine in the art and is taught in, for example, Current Protocols in Molecular Biology, John Wiley and Sons.

Real-time quantitative (PCR) can be conveniently accomplished using the commercially available iQ Multi-Color Real Time PCR Detection System, available from BioRAD.

Real-time Quantitative PCR Analysis of Ha-ras mRNA Levels

Quantitation of mRNA levels was determined by real-time quantitative PCR using the iQ Multi-Color Real Time PCR Detection System (BioRAD) according to the manufacturers instructions.

Real-time Quantitative PCR is a technique well known in the art and is taught in for example Heid et al. Real time quantitative PCR, Genome Research (1996), 6: 986-994.

Platinum Quantitative PCR SuperMix UDG 2× PCR master mix was obtained from Invitrogen cat# 11730. Primers and TaqMan® probes were obtained from MWG-Biotech AG, Ebersberg, Germany

Probes and primers to human Ha-ras were designed to hybridise to a human Ha-ras sequence, using published sequence information (GenBank accession number J00277, incorporated herein as SEQ ID NO:1).

For human Ha-ras the PCR primers were:

forward primer: 5′ gccggatgcaggaaggag 3′ (final concentration in the assay; 0.3 μM reverse primer: 5′ gctccagcagcccttcctt 3′ (final concentration in the assay; 0.3 μM)(SEQ ID NO: 81) and the PCR probe was: 5′ FAM-cgtccttccttcctcctccttccgtctg-TAMRA 3′ (final concentration in the assay; 0.1 μM)

Glyceraldehyde-3-phosphate dehydrogenase (GAPDH) mRNA quantity was used as an endogenous control for normalizing any variance in sample preparation.

The sample content of human GAPDH mRNA was quantified using the human GAPDH ABI Prism Pre-Developed TaqMan Assay Reagent (Applied Biosystems cat. no. 4310884E) according to the manufacturers instructions.

For quantification of mouse GAPDH mRNA the following primers and probes were designed: Sense primer 5′aaggctgtgggcaaggtcatc 3′ (0.3 μM final concentration), antisense primer 5′ gtcagatccacgacggacacatt (0.6 μM final concentration), TaqMan probe 5′ FAM-gaagctcactggcatggcatggccttccgtgttc-TAMRA 3′ (0.2 μM final concentration).

Real Time PCR

The cDNA from the first strand synthesis performed as described in example 8 was diluted 2-20 times, and analyzed by real time quantitative PCR. The primers and probe were mixed with 2× Platinum Quantitative PCR SuperMix UDG (cat. # 11730, Invitrogen) and added to 3.3 μl cDNA to a final volume of 25 μl. Each sample was analysed in triplicates. Assaying 2 fold dilutions of a cDNA that had been prepared on material purified from a cell line expressing the RNA of interest generated standard curves for the assays. Sterile H₂O was used instead of cDNA for the no template control. PCR program: 50° C. for 2 minutes, 95° C. for 10 minutes followed by 40 cycles of 95° C., 15 seconds, 60° C., 1 minutes. Relative quantities of target mRNA sequence were determined from the calculated Threshold cycle using the iCycler iQ Real-time Detection System software.

### Example 8

### In Vitro Analysis: Northern Blot Analysis of Ha-Ras mRNA Levels

Northern blot analysis was carried out by procedures well known in the art essentially as described in Current Protocols in Molecular Biology, John Wiley & Sons.

The hybridisation probe was obtained by PCR-amplification of a 381 bp fragment from 15PC3 cDNA obtained by reverse transcription PCR as described in example 8. The reaction was carried out using primers 5′ aatctcggcaggctcaggac 3′ (forward) and 5′ gggatgttcaagacagtctgtgc 3′ (reverse) at 0,5 μM final concentration each, 200 nM each dNTP, 1,5 mM MgCl₂ and Platinum Taq DNA polymerase (Invitrogen cat. no. 10966-018). The DNA was amplified for 40 cycles on a Perkin Elmer 9700 thermocycler using the following program: 94° C. for 2 min. then 40 cycles of 94° C. for 30 sec. and 72° C. for 30 sec. with a decrease of 0.5° C. per cycle followed by 72° C. for 7 min.

The amplified PCR product was purified using S-400 MicroSpin columns (Amersham Pharmacia Biotech cat. no. 27-5140-01) according to the manufacturers instructions and quantified by spectrophotometry.

The hybridisation probe was labelled using Redivue™ [α-³²P]dCTP 3000 Ci/mmol (Amersham Pharmacia Biotech cat. no. M 0005) and Prime-It RmT labeling kit (Stratagene cat. no. 300392) according to the manufacturers instructions and the radioactively labeled probe was purified using S-300 MicroSpin columns (Amersham Pharmacia Biotech cat. no. 27-5130-01). Before use, the probe was denatured at 96° C. and immediately put on ice.

Samples of 1-5 μg of total RNA purified as described in example 7 were denatured and size separated on a 2.2 M formaldehyde/MOPS agarose gel. RNA was transferred to positively charged nylon membrane by downward capillary transfer using the TurboBlotter (Schleicher & Schuell) and the RNA was immobilised to the membrane by UV crosslinking using a Stratagene crosslinker. The membrane was prehybridised in ExpressHyb Hybridization Solution (Clontech cat. No. 8015-1) at 60° C. and the probe was subsequently added for hybridisation. Hybridisation was carried out at 60° C. and the blot was washed with low stringency wash buffer (2×SSC, 0.1% SDS) at room temperature and with high stringency wash buffer (0.1×SSC, 0.1% SDS) at 50° C. The blot was exposed to Kodak storage phosphor screens and scanned in a BioRAD FX molecular imager. Ha-ras mRNA levels were quantified by Quantity One software (BioRAD)

Equality of RNA sample loading was assessed by stripping the blot in 0.5% SDS in H₂O at 85° C. and reprobing with a labelled GAPDH (glyceraldehyde-3-phosphate dehydrogenase) probe obtained essentially as described above using the primers 5′ aac gga ttt ggt cgt att 3′ (forward) and 5′ taa gca gtt ggt ggt gca 3′ (reverse).

### Example 9

### In Vitro Analysis: Western Blot Analysis of Ha-Ras Protein Levels

Protein levels of Ha-ras can be quantitated in a variety of ways well known in the art, such as immunoprecipitation, Western blot analysis (immunoblotting), ELISA, RIA (Radio Immuno Assay) or fluorescence-activated cell sorting (FACS). Antibodies directed to Ha-ras can be identified and obtained from a variety of sources, such as Upstate Biotechnologies (Lake Placid, USA), Novus Biologicals (Littleton, Colo.), Santa Cruz Biotechnology (Santa Cruz, Calif.) or can be prepared via conventional antibody generation methods.

Western Blotting:

To measure the effect of treatment with antisense oligonucleotides against Ha-ras, protein levels of Ha-ras in treated and untreated cells were determined using western blotting. After treatment with oligonucleotide as described in example 5, cells were harvested in ice-cold lysis buffer (50 mM Tris, pH 6.8, 10 mM NaF, 10% glycerol, 2.5% SDS, 0.1 mM natrium-orthovanadate, 10 mM β-glycerol phosphate, 10 mM dithiothreitol (DTT), Complete protein inhibitor cocktail (Boehringer Mannheim)). The lysate was stored at −80° C. until further use. Protein concentration of the protein lysate was determined using the BCA Protein Assay Kit (Pierce) as described by the manufacturer.

SDS Gel Electrophoresis:

Protein samples prepared as described above were thawed on ice and denatured at 96° C. for 3 min. Samples were loaded on 1.0 mm 4-20% NuPage Tris-glycine gel (Invitrogen) and gels were run in TGS running buffer (BioRAD) in an Xcell II Mini-cell electrophoresis module (Invitrogen).

Semi-Dry Blotting:

After electrophoresis, the separated proteins were transferred to a polyvinyliden difluoride (PVDF) membrane by semi-dry blotting. The blotting procedure was carried out in a Semi-Dry transfer cell (CBS Scientific Co.) according to the manufacturers instructions. The membrane was stained with amidoblack to visualise transferred protein and was stored at 4° C. until further use.

Immunodetection:

To detect the desired protein, the membrane was incubated with either polyclonal or monoclonal antibodies against the protein.

The membrane was blocked in blocking buffer (5% skim milk powder dissolved in PBST-buffer (PBS+0.1% Tween-20)), washed briefly in PBS-buffer and incubated with primary antibody in blocking buffer at room temperature. The following primary and secondary antibodies and concentrations/dilutions were used:

Polyclonal rabbit anti-human H-ras antibody (cat. # sc-520, Santa Cruz) 1:200

Monoclonal mouse anti-human tubulin Ab-4 (cat.# MS-719-P1, NeoMarkers) 1:500

Peroxidase-conjugated Swine Anti-Rabbit Immunoglobulins (code no. P0399, DAKO) 1:3000

Peroxidase-conjugated Goat Anti-Mouse Immunoglobulins (code no. P0447, DAKO) 1:1000

After incubation with the primary antibody the membrane was washed briefly in PBS followed by 3 additional 10 minutes washes in PBST with agitation at room temperature and incubated with a peroxidase conjugated secondary antibody in blocking buffer at room temperature. The membrane was then washed in PBS followed by 3 additional 10 minutes washes in PBST with agitation at room temperature. After the last wash the membrane was incubated with ECL⁺ Plus reagent (Amersham) and chemiluminescens was detected using VersaDoc chemiluminescens detection system (BioRAD) or X-omat film (Kodak). The membrane was stripped in ddH₂O by incubation for 1 minute at 96° C. After stripping, the membrane was put in PBS and stored at 4° C.

### Example 10

### In Vitro Analysis: Antisense Inhibition of Human Ha-Ras Expression by Oligomeric Compound

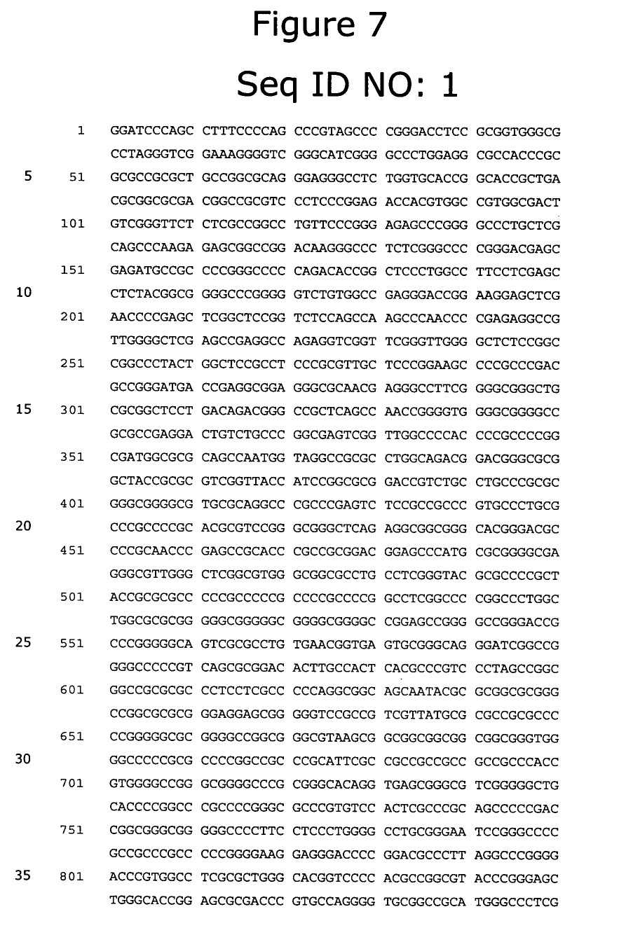

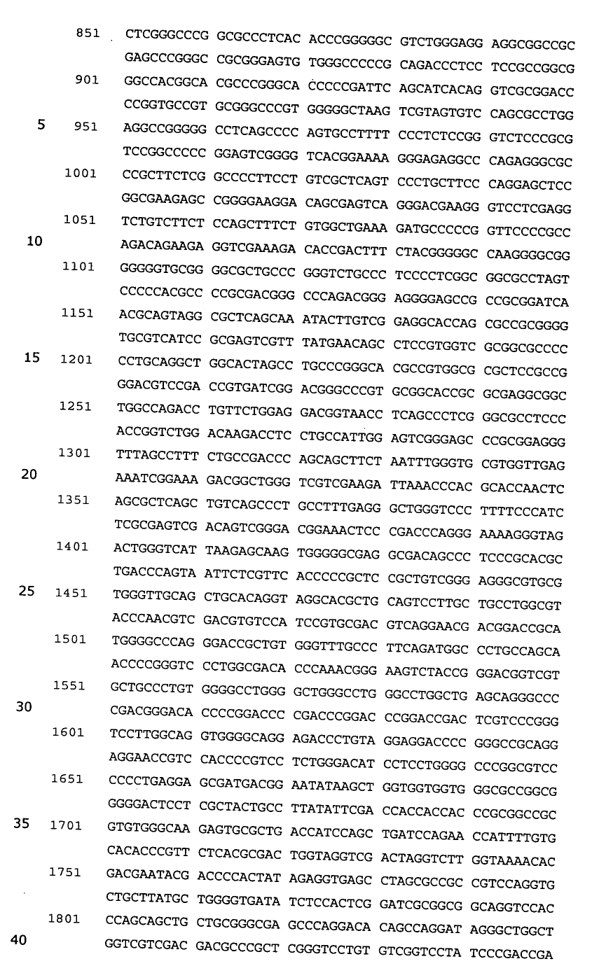

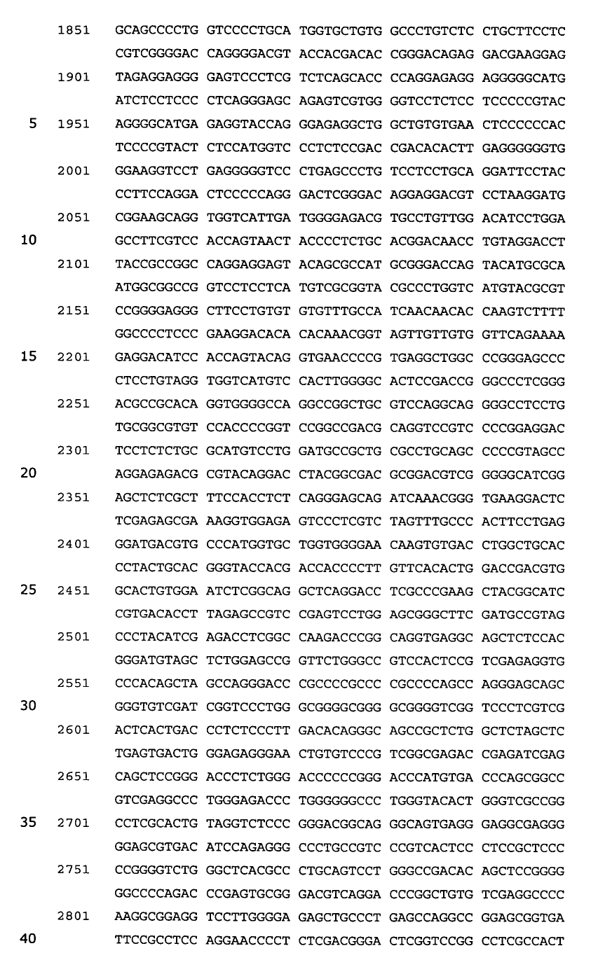

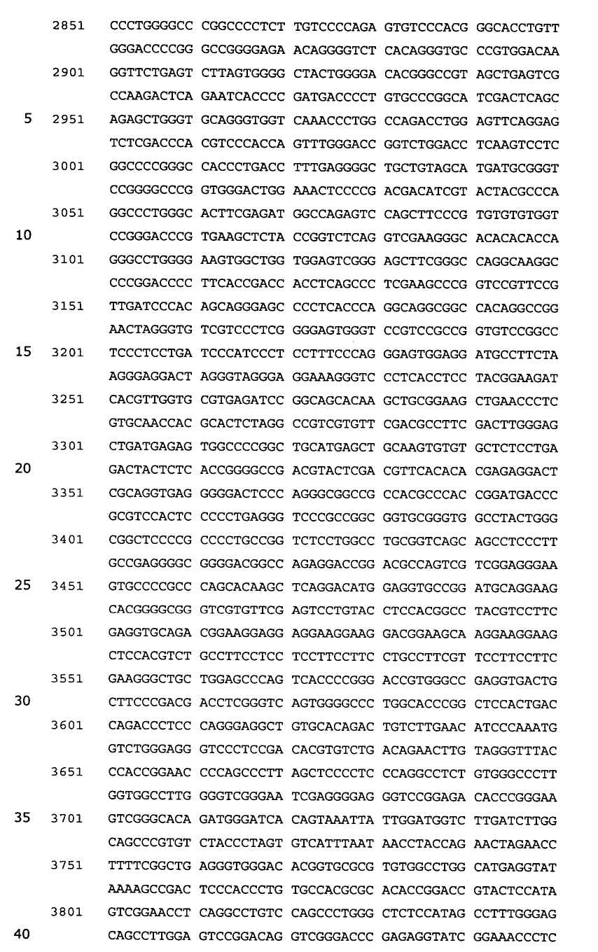

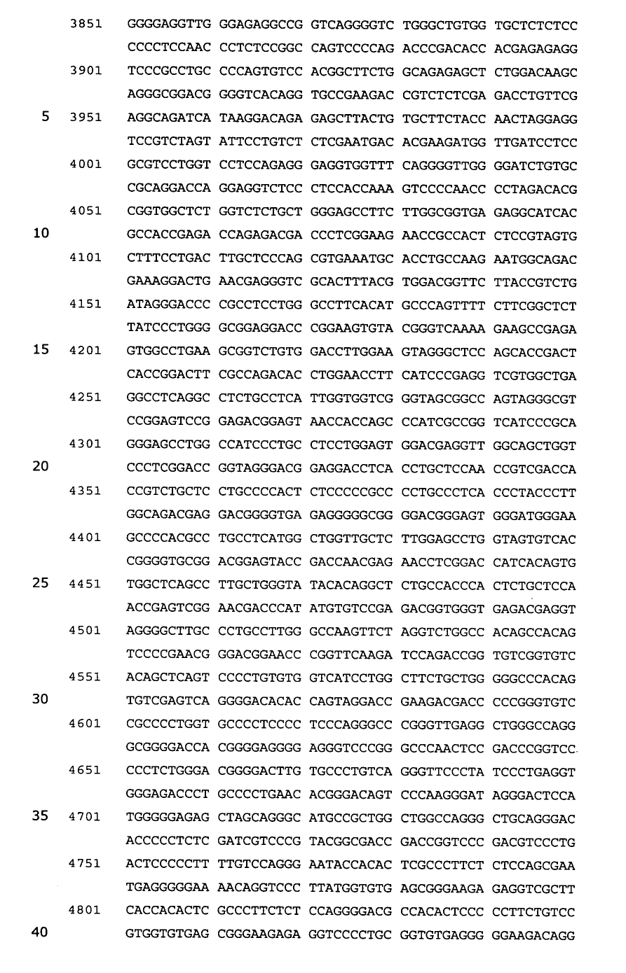

In accordance with the present invention, a series of oligonucleotides were designed to target different regions of the human Ha-ras RNA, using published sequences (GenBank accession number J00277, incorporated herein as SEQ ID NO: 1, FIG. 7). The oligomeric compounds with 16 nucleotides in length are shown in Table 1 having SEQ ID NO and number and specific designs A, B and C. Some of the compounds do also have a internal “CUR” number. “Target site” indicates the first nucleotide number on the particular target sequence to which the oligonucleotide binds. Table 2 shows low IC50 of four compounds.

Table 1 Oligomeric Compounds of the Invention

Oligomeric compounds were evaluated for their potential to knockdown Ha-ras mRNA in 15PC3 cells. The data are presented as percentage downregulation relative to mock transfected cells. Transcript steady state was monitored by Real-time PCR and normalised to the GAPDH transcript steady state. Note that all LNA C are 5′-Methyl-Cytosine.

| | | | Internal | Specific design of Oligomeric | |
|---|---|---|---|---|---|
| | | | NO | compound | |
| | | | & | Capital letters bold β-D-oxy-LNA | |
| | | Oligomeric compound | ID NO + | S = phosphorthioate | |
| | Target | Sequence | Design | O = —O—P(O)₂—O— | % Inhibition |
| SEQ ID NO | site | 5′-3′ | NO | Small letters DNA sugar | at 25 nM oligo |

| | | | | | |
|---|---|---|---|---|---|
| 2 | 1742 | ATTCGTCCACAAAATG | CUR2709 | A_{S}T_{S}T_{S}C_{S}g_{S}t_{S}c_{S}c_{S}a_{S}c_{S}a_{S}a_{S}A_{S}A_{S}T_{S}G | 29 |
| | (260 K- | | 2A | | |
| | ras) | | | | |
| | | | 2B | A_{S}T_{S}T_{S}C_{S}g_{S}t_{S}c_{S}c_{S}a_{S}c_{S}a_{S}a_{S}A_{S}A_{S}T_{S}g | |
| | | | 2C | A_{O}T_{O}T_{O}C_{O}g_{S}t_{S}c_{S}c_{S}a_{S}c_{S}a_{S}a_{S}A_{O}A_{O}T_{O}G | |
| 3 | 1733 | CAAAATGGTTCTGGAT | CUR2710 | C_{S}A_{S}A_{S}A_{S}a_{S}t_{S}g_{S}g_{S}t_{S}t_{S}c_{S}t_{S}G_{S}G_{S}A_{S}T | 60 |
| | (323 N- | | 3A | | |
| | ras) | | | | |
| | | | 3B | C_{S}A_{S}A_{S}A_{S}a_{S}t_{S}g_{S}g_{S}t_{S}t_{S}c_{S}t_{S}G_{S}G_{S}A_{S}t | |
| | | | 3C | C_{O}A_{O}A_{O}A_{O}a_{S}t_{S}g_{S}g_{S}t_{S}t_{S}c_{S}t_{S}G_{O}G_{O}A_{O}T | |
| 4 | 1745 | CGTATTCGTCCACAAA | CUR2711 | C_{S}G_{S}T_{S}A_{S}t_{S}t_{S}c_{S}g_{S}t_{S}c_{S}c_{S}a_{S}C_{S}A_{S}A_{S}A | 67 |
| | (263 K- | | 4A | | |
| | ras) | | | | |
| | | | 4B | C_{S}G_{S}T_{S}A_{S}t_{S}t_{S}c_{S}g_{S}t_{S}c_{S}c_{S}a_{S}C_{S}A_{S}A_{S}a | |
| | | | 4C | C_{O}G_{O}T_{O}A_{O}t_{S}t_{S}c_{S}g_{S}t_{S}c_{S}c_{S}a_{S}C_{O}A_{O}A_{O}A | |
| 5 | 2158 | CACACACAGGAAGCCC | CUR2712 | C_{S}A_{S}C_{S}A_{S}c_{S}a_{S}c_{S}a_{S}g_{S}g_{S}c_{S}c_{S}G_{S}C_{S}C_{S}C | 62 |
| | | | 5A | | |
| | | | 5B | C_{S}A_{S}C_{S}A_{S}c_{S}a_{S}c_{S}a_{S}g_{S}g_{S}a_{S}a_{S}G_{S}C_{S}C_{S}c | |
| | | | 5C | C_{O}A_{O}C_{O}A_{O}c_{S}a_{S}c_{S}a_{S}g_{S}g_{S}a_{S}a_{S}G_{O}C_{O}C_{O}C | |
| 6 | 3701 | CCCATCTGTGCCCGAC | CUR2713 | C_{S}C_{S}C_{S}A_{S}t_{S}c_{S}t_{S}g_{S}t_{S}g_{S}c_{S}c_{S}C_{S}G_{S}A_{S}C | 90 |
| | | | 6A | | |
| | | | 6B | C_{S}C_{S}C_{S}A_{S}t_{S}c_{S}t_{S}g_{S}t_{S}g_{S}c_{S}c_{S}C_{S}G_{S}A_{S}v | |
| | | | 6C | C_{O}C_{O}C_{O}A_{O}t_{S}c_{S}t_{S}g_{S}t_{S}g_{S}c_{S}c_{S}C_{O}G_{O}A_{O}C | |
| 7 | 2168 | TGATGGCAAACACACA | CUR2714 | T_{S}G_{S}A_{S}T_{S}g_{S}g_{S}c_{S}a_{S}a_{S}a_{S}c_{S}a_{S}C_{S}A_{S}C_{S}A | 63 |
| | (491 N- | | 7A | | |
| | ras) | | | | |
| | | | 7B | T_{S}G_{S}A_{S}T_{S}g_{S}g_{S}c_{S}a_{S}a_{S}a_{S}c_{S}a_{S}C_{S}A_{S}C_{S}A | |
| | | | 7C | T_{O}G_{O}A_{O}T_{O}g_{S}g_{S}c_{S}a_{S}a_{S}a_{S}c_{S}a_{S}C_{O}A_{O}C_{O}A | |
| 8 | 2182 | AGACTTGGTGTTGTTG | CUR2715 | A_{S}G_{S}A_{S}C_{S}t_{S}t_{S}g_{S}g_{S}t_{S}g_{S}t_{S}t_{S}G_{S}T_{S}T_{S}G | 57 |
| | | | 8A | | |
| | | | 8B | A_{S}G_{S}A_{S}C_{S}t_{S}t_{S}g_{S}g_{S}t_{S}g_{S}t_{S}t_{S}G_{S}T_{S}T_{S}g | |
| | | | 8C | A_{O}G_{O}A_{O}C_{O}t_{S}t_{S}g_{S}g_{S}t_{S}g_{S}t_{S}t_{S}G_{O}T_{O}T_{O}G | |
| 9 | 2383 | GTCCTTCACCCGTTTG | CUR2714 | G_{S}T_{S}C_{S}C_{S}t_{S}t_{S}c_{S}a_{S}c_{S}c_{S}c_{S}g_{S}T_{S}T_{S}T_{S}G | 67 |
| | | | 9A | | |
| | | | 9B | G_{S}T_{S}C_{S}C_{S}t_{S}t_{S}c_{S}a_{S}c_{S}c_{S}c_{S}g_{S}T_{S}T_{S}T_{S}G | |
| | | | 9C | G_{O}T_{O}C_{O}C_{O}t_{S}t_{S}c_{S}a_{S}c_{S}c_{S}c_{S}g_{S}T_{O}T_{O}T_{O}g | |
| 10 | 2393 | CGTCATCCGAGTCCTT | CUR2717 | C_{S}G_{S}T_{S}C_{S}a_{S}t_{S}c_{S}c_{S}g_{S}a_{S}g_{S}t_{S}C_{S}C_{S}T_{S}T | 66 |
| | | | 10A | | |
| | | | 10B | C_{S}G_{S}T_{S}C_{S}a_{S}t_{S}c_{S}c_{S}g_{S}a_{S}g_{S}t_{S}C_{S}C_{S}T_{S}t | |
| | | | 10C | C_{O}G_{O}T_{O}C_{O}a_{S}t_{S}c_{S}c_{S}g_{S}a_{S}g_{S}t_{S}C_{O}C_{O}T_{O}T | |
| 11 | 2431 | AGCCAGGTCACACTTG | CUR2718 | A_{S}G_{S}C_{S}C_{S}a_{S}g_{S}g_{S}t_{S}c_{S}a_{S}c_{S}a_{S}C_{S}T_{S}T_{S}G | 49 |
| | | | 11A | | |
| | | | 11B | A_{S}G_{S}C_{S}C_{S}a_{S}g_{S}g_{S}t_{S}c_{S}a_{S}c_{S}a_{S}C_{S}T_{S}T_{S}g | |
| | | | 11C | A_{O}G_{O}C_{O}C_{O}a_{S}g_{S}g_{S}t_{S}c_{S}a_{S}c_{S}a_{S}C_{O}T_{O}T_{O}G | |
| 12 | 2453 | GCCGAGATTCCACAGT | CUR2719 | G_{S}C_{S}C_{S}G_{S}a_{S}g_{S}a_{S}t_{S}t_{S}c_{S}c_{S}a_{S}C_{S}A_{S}G_{S}T | 77 |
| | | | 12A | | |
| | | | 12B | G_{S}C_{S}C_{S}G_{S}a_{S}g_{S}a_{S}t_{S}t_{S}c_{S}c_{S}a_{S}C_{S}A_{S}G_{S}t | |
| | | | 12C | G_{O}C_{O}C_{O}G_{O}a_{S}g_{S}a_{S}t_{S}t_{S}c_{S}c_{S}a_{S}C_{O}A_{O}G_{O}T | |
| 13 | 3228 | CATCCTCCACTCCCTG | CUR2720 | C_{S}A_{S}T_{S}C_{S}c_{S}t_{S}c_{S}c_{S}a_{S}c_{S}t_{S}c_{S}C_{S}C_{S}T_{S}G | 68 |
| | (629 K- | | 13A | | |
| | ras) | | | | |
| | | | 13B | C_{S}A_{S}T_{S}C_{S}c_{S}t_{S}c_{S}c_{S}a_{S}c_{S}t_{S}c_{S}C_{S}C_{S}T_{S}G | |
| | | | 13C | C_{O}A_{O}T_{O}C_{O}c_{S}t_{S}c_{S}c_{S}a_{S}c_{S}t_{S}c_{S}C_{O}C_{O}T_{O}g | |
| 14 | 3253 | ATCTCACGCACCAACG | CUR2721 | A_{S}T_{S}C_{S}T_{S}c_{S}a_{S}c_{S}g_{S}c_{S}a_{S}c_{S}c_{S}A_{S}A_{S}C_{S}G | 89 |
| | | | 14A | | |
| | | | 14B | A_{S}T_{S}C_{S}T_{S}c_{S}a_{S}c_{S}g_{S}c_{S}a_{S}c_{S}c_{S}A_{S}A_{S}C_{S}g | |
| | | | 14C | A_{O}T_{O}C_{O}T_{O}c_{S}a_{S}c_{S}g_{S}c_{S}a_{S}c_{S}c_{S}A_{O}A_{O}C_{O}G | |
| 15 | 3506 | TCCTCCTTCCGTCTGC | CUR2722 | T_{S}C_{S}C_{S}T_{S}c_{S}c_{S}t_{S}t_{S}c_{S}c_{S}g_{S}t_{S}C_{S}T_{S}G_{S}C | 99 |
| | | | 15A | | |
| | | | 15B | T_{S}C_{S}C_{S}T_{S}c_{S}c_{S}t_{S}t_{S}c_{S}c_{S}g_{S}t_{S}C_{S}T_{S}G_{S}c | |
| | | | 15C | T_{O}C_{O}C_{O}T_{O}c_{S}c_{S}t_{S}t_{S}c_{S}c_{S}g_{S}t_{S}C_{O}T_{O}G_{O}C | |
| 16 | 1610 | GGTCTCCTGCCCCACC | | | |
| 17 | 1626 | CGGGGTCCTCCTACAG | | | |
| 18 | 1642 | TCAGGGGCCTGCGGCC | | | |
| 19 | 1658 | ATTCCGTCATCGCTCC | | | |
| 20 | 1674 | ACCACCACCAGCTTAT | | | |
| 21 | 1690 | CACACCGCCGGCGCCC | | | |
| 22 | 1706 | TCAGCGCACTCTTGCC | | | |
| 23 | 1738 | GTCCACAAAATGGTTC | | | |
| 24 | 1754 | TAGTGGGGTCGTATTC | | | |
| 25 | 2037 | CGGTAGGAATCCTCTA | | | |
| 26 | 2053 | AATGACCACCTGCTTC | | | |
| 27 | 2069 | GGCACGTCTCCCCATC | | | |
| 28 | 2085 | TCCAGGATGTCCAACA | | | |
| 29 | 2101 | CTCCTGGCCGGCGGTA | | | |
| 30 | 2117 | GCATGGCGCTGTACTC | | | |
| 31 | 2133 | CGCATGTACTGGTCCC | | | |
| 32 | 2149 | GAAGCCCTCCCCGGTG | | | |
| 33 | 2165 | TGGCAAACACACACAG | | | |
| 34 | 2181 | GACTTGGTGTTGTTGA | | | |
| 35 | 2197 | GTGGATGTCCTCAAAA | | | |
| 36 | 2213 | TCTGCTCCCTGTACTG | | | |
| | Exon- | | | | |
| | exon | | | | |
| 37 | 2382 | TCCTTCACCCGTTTGA | | | |
| 38 | 2398 | GGGCACGTCATCCGAG | | | |
| 39 | 2414 | TCCCCACCAGCACCAT | | | |
| 40 | 2430 | GCCAGGTCACACTTGT | | | |
| 41 | 2446 | TTCCACAGTGCGTGCA | | | |
| 42 | 2462 | CCTGAGCCTGCCGAGA | | | |
| 43 | 2478 | TAGCTTCGGGCGAGGT | | | |
| 44 | 2494 | GATGTAGGGGATGCCG | | | |
| 45 | 2510 | TCTTGGCCGAGGTCTC | | | |
| 46 | 2526 | TCCACTCCCTGCCGGG | | | |
| | Exon- | | | | |
| | exon | | | | |
| 47 | 3239 | CGTGTAGAAGGCATCC | | | |
| 48 | 3255 | GGATCTCACGCACCAA | | | |
| 49 | 3271 | CGCAGCTTGTGCTGCC | | | |
| 50 | 3287 | AGGAGGGTTCAGCTTC | | | |
| 51 | 3303 | CGGGGCCACTCTCATC | | | |
| 52 | 3319 | TTGCAGCTCATGCAGC | | | |
| 53 | 3335 | TCAGGAGAGCACACAC | | | |
| 54 | 3459 | CTGAGCTTGTGCTGCG | | | |
| 55 | 3475 | CCGGCACCTCCATGTC | | | |
| 56 | 3491 | CACCTCCTTCCTGCAT | | | |
| 57 | 3507 | CTCCTCCTTCCGTCTG | | | |
| 58 | 3523 | CTTCCGTCCTTCCTTC | | | |
| 59 | 3539 | CTTCCTTCCTTCCTTG | | | |
| 60 | 3555 | CTGGGCTCCAGCAGCC | | | |
| 61 | 3571 | CACGGTCCCGGGGTGA | | | |
| 62 | 3587 | TGCAGTCACCTCGGCC | | | |
| 63 | 3603 | CCTCCCTGGGAGGGTC | | | |
| 64 | 3619 | GACAGTCTGTGCACAG | | | |
| 65 | 3635 | CATTTGGGATGTTCAA | | | |
| 66 | 3651 | GCTGGGGTTCCGGTGG | | | |
| 67 | 3667 | GGGAGGGGAGCTAAGG | | | |
| 68 | 3683 | GGGCCCACAGAGGCCT | | | |
| 69 | 3699 | CATCTGTGCCCGACAA | | | |
| 70 | 3715 | TAATTTACTGTGATCC | | | |
| 71 | 3731 | TTTCAAGACCATCCAA | | | |
| 72 | 1722 | TGGATCAGCTGGATGG | | | |
| 73 | 1690 | CACACCGTCGGCGCCC | | | |
| 74 | 2101 | CTCCAGGCCGGCGGTA | | | |

Additional compounds are precented in table 3, 4 and 5 and in FIG. 2.

### Table 2 IC50 (nM) of the LNA (β-D-oxy-LNA) Containing Oligomeric Compounds

Oligomeric compounds were evaluated for their potential to knockdown Ha-ras mRNA in 15PC3 cells. Transcript steady state was monitored by Real-time PCR and normalised to the GAPDH transcript steady state. Note that all LNA C are 5′-Methyl-Cytosine.

| Internal number & | |
|---|---|
| SEQ ID NO | IC50 in 15PC3 |
| CUR2710 (3A) | <0.5 |
| CUR2713 (6A) | <0.5 |
| CUR2721 (14A) | <1 |
| CUR2722 (15A) | <0.5 |
| CUR2524 (76A) | <1 |

In comparison to the very potent molecules in Table 2, it has been reported that a 20-mer phosphorthioate targeting Ha-ras, named ISIS2503, has a IC₅₀ of 45 nM (Bennett et al.(1996) Antisense therapeutics, Humana Press, Totowa, N.J., 13-17).

As showed In table 1 and 2, SEQ ID NO 3, 4, 5, 6, 7, 8, 9, 10, 11, 12, 13, 14, 15 and 77 demonstrated at least 30% inhibition of Ha-ras expression at 25 nM in these experiments and are therefore preferred.

Compounds of particular interest are 3A, 4A, 5A, 6A, 7A, 8A, 9A, 10A, 11A, 12A, 13A, 14A, 15A and 76A.

### Example 11

### Apoptosis Induction by LNA Antisense Oligomeric Compounds Targeting HA-Ras

Measurement of apoptosis using BD™ cytometric bead array (CBA) (cat 557816).

Cells were transfected using lipofectamine 2000 as described (see Example 5). 24 h following transfection, the cells from the supernatant was spun down and the adherent cells were trypsionised and spun down. The cell pellet was resuspended/washed in PBS and counted to bring cell concentration to 2×10⁶ cells/ml lysis buffer containing protease inhibitors. The procedure was proceeded as described by manufacturer with the following modifications. When cells were lysed, they were lysed for 40 min and vortexed with a 10 min interval. 1×10⁵ cells were incubated with Caspase 3 beads, mixed briefly and incubated for 1 h at room temperature, before they were analysed by flow cytometri. The data were analysed using the BD™ CBA software, transferred to Excel where all data were related to mock (which is set to one). (see FIG. 6 upper panel).

Furthermore, an oligo directed against H-Ras or its mismatch control was tested (in two different designs (alfa-L-LNA versus oxy-LNA; Compounds 2776, 2778, 2742 and 2744 see table 5) in an in vitro caspase 3 assay (CBA). The matched and the mismatched oxy LNA induced apoptosis to similar extend (when compared to mock) as the matched alfa-L-LNA, whereas the mismatched alfa-L-LNA oligo did not induce apoptosis noteworthy. The data presented here clearly demonstrate that downregulation of H-Ras by antisense inhibition induced apoptosis (Caspase 3). (see FIG. 6 lower panel)

### Example 12

### Antisense Oligonucleotide Inhibition of Ha-Ras in Proliferating Cancer Cells

Cells were seeded to a density of 12000 cells per well in white 96 well plate (Nunc 136101) in DMEM the day prior to transfection. The next day cells were washed once in prewarmed OptiMEM followed by addition of 72 μl OptiMEM containing 5 μg/ml Lipofectamine2000 (In vitrogen). Cells were incubated for 7 min before adding 18 μl oligonucleotides diluted in OptiMEM. The final oligonucleotide concentration ranged from 5 nM to 100 nM. After 4 h of treatment, cells were washed in OptiMEM and 100 μl serum containing DMEM was added. Following oligo treatment cells were allowed to recover for the period indicated, viable cells were measured by adding 20 μl the tetrazolium compound [3-(4,5-dimethyl-2-yl)-5-(3-carboxymethoxyphenyl)-2-(4-sulfophenyl)-2H-tetrazolium, inner salt; MTS] and an electron coupling reagent (phenazine ethosulfate; PES) (CellTiter 96® AQᵤₑₒᵤₛ One Solution Cell Proliferation Assay, Promega). Viable cells were measured at 490 nm in a Powerwave (Biotek Instruments). Growth rate (ΔOD/h) were plotted against oligo concentration.

### Example 13

### Measurement of Ploidy (Cell Cycle) and DNA Degradation (Apoptosis) of Cells Following Treatment with Oligomeric Compounds Targeting Ha-Ras

The late stage in the apoptotic cascade leads to large numbers of small fragments of DNA which can be analysed by propidium iodide staining of the cells, furthermore, propidium iodide staining can be used to asses ploidy in treated cells. To assess ploidy/apoptosis of cells treated with oligomeric compound directed against Ha-ras, cells were washed in PBA and fixed for 1 h in 70% EtOH at 4° C. After treatment with 50 μg/ml RNAse (Sigma) for 20 min at room temperature cells were washed with PBS and incubated with 40 μg/ml propidium iodide (Sigma or BD) for 30 min. All samples were analysed using fluorescence activated cell sorter (FACSCalibur, Becton Dickinson) and Cell Quest software. In the DNA histogram the hypodiploid or the sub-G1 peak represented the apoptotic cells.

### Example 14

### Measurement of Changes in the Mitochondrial Membrane Potential of Cells Following Treatment with Oligomeric Compounds Targeting Ha-Ras

To measure changes in the mitochondrial membrane potential the MitoSensor™ reagent method (Becton Dickinson, Cat # K2017-1) was used. MitoSensor™ reagent is taken up by healthy cells, in which it forms aggregates that emit red fluorescence. Upon apoptosis the mitochondrial membrane potential changes and does not allow the reagent to aggregate within the mitochondria and therefore it remains in the cytoplasm in its monomeric form where it emits green fluorescence. Cells treated with oligomeric compounds directed against Ha-ras were washed and incubated in MitoSensor Reagent diluted in Incubation buffer as described by manufacturer. Changes in membrane potential following oligo treatment was detected by fluorescence activated cell sorter (FACSCalibur, Becton Dickinson) and by the use of Cell Quest software.

### Example 15

### Inhibition of Capillary Formation of Endothelial Cells Following Antisense Oligo Treatment

Endothelial monolayer cells (e.g. HUVEC) were incubated with antisense oligos directed against Ha-ras. Tube formation was analysed by either of the two following methods. The first method was the BD BioCoat angiogenesis tube formation system. Cells were transfected with oligos as described (example 5). Transfected cells were seeded at 2×10⁴ cells/96 well onto matrigel polymerized BD Biocoat angiogeneis plates. The plates were incubated for the hours/days indicated with or without PMA (5-50 nM), VEGF (20-200 ng/ml), Suramin or vechicle. The plates were stained with Cacein AM as stated by the manufacturer and images were taken. Total tube length was measured using MetaMorph. Althernatively, cells were seeded in rat tail type I collagen (3 mg/ml, Becton Dickinson) in 0.1 volumen of 10×DMEM, neutralised with sterile 1 M NaOH and kept on ice or in matrigel. Cells were added to the collagen suspension at a final concentration of 1×10⁶ cells/ml collagen. The cell-collagen mixture was added to 6-well or 35 mm plates and placed in a humidified incubator at 37° C. When geled 3 ml of culture medium plus an extra 10% FBS were added and cells were allow to form capillary-like vascular tubes over the period indicated in the presence or absence of PMA (16 nM), VEGF (50 ng/ml). Tube formation was quantified following cryostat sectioning of the gels and examination of sections by phase-contrast microscopy.

### Example 16

### In Vivo Model: Tumour Growth Inhibition of Human Tumour Cells Grown In Vivo by Systemic Treatment with Oligomeric Compound

Female NMRI athymic nude mice of 6 weeks old were purchased from M&B, Denmark and allowed to acclimatize for at least one week before entering experiments. Human cancer cells typically 10⁶ cells suspended in 300 μl matrigel (BD Bioscience), were subcutaneously injected into the flanks of 7-8 week old NMRI athymic female nude mice. When the tumour growth was established, typically 7-12 days post tumour cell injection; different antisense oligonucleotides were administrated at 5 mg/kg/day for up to 28 days using ALZET osmotic pumps implanted subcutaneously. Prior to dorsal implantation the pumps were incubated overnight at room temperature in sterile PBS to start the pumps. Control animals received saline alone for the same period. Each experimental group included at least 5 mice. Anti-tumour activities were estimated by the inhibition of tumour volume. Tumour growth was followed regularly by measuring 2 perpendicular diameters. Tumour volumes were calculated according to the formula (π×L×D²/6), where L represents the largest diameter and D the tumour diameter perpendicular to L. At the end of treatment the animals were sacrificed and tumour weights were measured. Mean tumour volume and weights of groups were compared using Mann-Whitney's test. All analysis was made in SPSS version 11.0 for windows.

Optimally, a Western blot analysis may also be performed to measure if the antisense oligonucleotides have an inhibitory effect on protein levels. At the end of treatment period mice were therfore anaesthetised and the tumours were excised and immediately frozen in liquid nitrogen.

The tumours were homogenized in lysis buffer (i.e. 20 mM Tris-Cl [pH 7.5]; 2% Triton X-100; 1/100 vol. Protease Inhibitor Cocktail Set III (Calbiochem); 1/100 vol. Protease Inhibitor Cocktail Set II (Calbiochem)) at 4° C. with the use of a motor-driven homogeniser. 500 μl lysis buffer was applied per 100 mg tumour tissue. Tumour lysates from each group of mice were pooled and centrifuged at 13.000 g for 5 min at 4° C. to remove tissue debris. Protein concentrations of the tumour extracts were determined using the BCA Protein Assay Reagent Kit (Pierce, Rockford).

The protein extracts (50-100 μg) were fractionated on a gradient SDS-PAGE gel spanning from 4-20% and transferred to PVDF membranes and visualized by aminoblack staining. The expression of Ha-ras was detected with anti-human Ha-ras antibody followed by horseradish peroxidase-conjugated anti-goat IgG (DAKO). Immunoreactivity was detected by the ECL Plus (Amersham biotech) and quantitated by a Versadoc 5000 lite system (Bio-Rad).

### Example 17

### In Vivo Model: Tumor Growth Inhibition of Human Tumour Fragments Transplanted in Nude Mice After Intraperetoneal Treatment with LNA Antisense Oligos

Tumour growth inhibiting activity of LNA antisense oligonucleotides was tested in xenotransplanted athymic nude mice, NMRI nu/nu, from Oncotest's (Freiburg, Germany) breeding colony. Human tumour fragments from breast (MDA MB 231), prostate (PC3) or lung tumours (LXFE 397, Oncotest) were obtained from xenografts in serial passage in nude mice. After removal of tumors from donor mice, they were cut into fragments (1-2 mm diameter) and placed in RPMI 1640 culture medium until subcutaneous implantation. Recipient mice were anaesthetized by inhalation of isoflurane. A small incision was made in the skin of the back. The tumor fragments (2 fragments per mouse) were transplanted with tweezers. MDA MB 231 and LXFE 397 tumors were tarnsplanted in female mice, PC3 tumors were transplanted in male mice. When a mean tumour diameter 4-6 mm was reached, animals were randomized and treated with oligonucleotides at 20 mg/kg intraperetoneally once a day for three weeks excluding weekends. A vehicle (saline) and positive control group (Taxol, 20 mg/kg/day) were included in all experiments. All groups consisted of 6 mice. The tumour volume was determined by two-dimensional measurement with a caliper on the day of randomization (Day 0) and then twice weekly. Tumor volumes were calculated according to the formula: (a×b²)×0.5 where a represents the largest and b the perpendicular tumor diameter. Mice were observed daily for 28 days after randomization until tumour volume was doubled. Mice were sacrificed when the tumour diameters exceeded 1.6 cm. For the evaluation of the statistical significance of tumour inhibition, the U-test by Mann-Whitney-Wilcoxon was performed. By convention, p-values <0.05 indicate significance of tumor inhibition.

### Example 18

### Biodistribution of Oligonucleotides in Mice

Female NMRI athymic nude mice of 6 weeks old were purchased from M&B, Denmark and allowed to acclimatize for at least one week before entering experiments. Human cancer cells typically 10⁶ cells suspended in 300 μl matrigel (BD Bioscience) were subcutaneously injected into the flanks of 7-8 week old NMRI athymic female nude mice. When tumour growth was evident, tritium labelled oligonucleotides were administrated at 5 mg/kg/day for 14 days using ALZET osmotic pumps implanted subcutaneously. The oligonucleotides were tritium labeled as described by Graham M J et al. (J Pharmacol Exp Ther 1998; 286(1): 447-458). Oligonucleotides were quantitated by scintillation counting of tissue extracts from all major organs (liver, kidney, spleen, heart, stomach, lungs, small intestine, large intestine, lymph nodes, skin, muscle, fat, bone, bone marrow) and subcutaneous transplanted human tumour tissue.

### Example 19

### In Vitro Superiority of LNA Containing Oligomeric Compounds

Human prostate cancer cell line 15PC3 was maintained as described in example 4. Cells were transfected using the lipid transfection reagent DAC-30 (Eurogentec) as described in Ten Asbroek et al.(2000), Polymorphisms in the large subunit of human RNA polymerase II as target for allele-specific inhibition. Nucleic Acid Research 28: 1133-1138. Oligo concentrations used for transfection were 200 nM, 400 nM and 800 nM final concentration. Expression levels of Ha-ras RNA was determined by Northern blot analysis using a protocol as described in Ten Asbroek et al.(2000), Polymorphisms in the large subunit of human RNA polymerase II as target for allele-specific inhibition (see FIG. 2). Nucleic Acid Research 28: 1133-1138. Hybridisation probes were generated by RT-PCR and subsequent cloning into pGEM-T Easy vector (Promega). The Ha-ras probe consisted of the sequence from position 1657-3485 (exon sequences only) of Seq ID NO. 1 (FIG. 7).

### Example 20

### In Vivo Superiority and Specificity of LNA Oligomeric Compounds Compared to Corresponding Phosphorothioates

Table 3 shows the antisense compound prepared for the In vivo superiority and specificity analysis.

TABLE 3
Oligonucleotides prepared for the In vivo superiority and specificity
analysis

| Seq ID | Cureon | | Sequence (Capital letters is β-D- |
|---|---|---|---|
| No | number/ | Length and design | oxy-LNA, s is phosphorothioate) |
| 75 | 75D | 16-mer fully thiolated | 5′-tₛcₛcₛgₛtₛcₛaₛtₛcₛgₛcₛtₛcₛcₛtₛc-3′ |
| | Cur2522* | | |
| | 75B | 16-mer | 5′-tₛCₛCₛGₛtₛcₛaₛtₛcₛgₛcₛtₛCₛCₛTₛc-3′ |
| | Cur2524 | LNA gapmer 3 + 3, fully | |
| | | thiolated | |
| 76A | 76A | 16-mer, LNA gapmer | 5′-tₛCₛAₛGₛtₛaₛaₛtₛaₛgₛcₛcₛCₛCₛAₛc-3′ |
| | Cur2525 | 3 + 3, fully thiolated, 5 | |
| | | mismatches | |

*The benchmark oligonucleotide: ISIS 2503 n-4 i.e the ISIS 2503 oligonucleotide which is made 4 bp shorter.

Tumor Growth Analysis

Two separate experiments were carried out. Female NMRI nude mice of 7-8 weeks old were obtained from M&B. Mice were kept 5 in each cage and allowed to acclimatize at least one week before entering experiments. Mice were injected subcutaneous with 10⁶ 15PC3 human prostate cancer cells suspended in 300 μl matrigel as previously described by K. Fleüiter. One week after tumor cell injection the anti-HaRas oligonucleotides, the mismatch control oligo and PBS were administrated subcutaneously for 14 days using ALZET osmotic pumps (model 1002). Prior to dorsal implantation the pumps were incubated overnight at room temperature in sterile PBS. Each group included 5-6 mice. Some mice carried two tumors. Tumor volumes were calculated according to the formula (π×L×D²/6), where L represents the largest diameter and D the tumor diameter perpendicular to L. Each tumor was regarded as one experimental unit. The experiments were blinded. After end treatment (14 days) mice were sacrificed and tumors were excised, freezed and kept for protein analysis. Tumors weights were also recorded.

Results

Tumor growth was almost inhibited by the fully thiolated 16-mer LNA gapmer containing 3 LNA's in each flank (Cur2524). This effect was demonstrated at 2.5 mg/kg/day (FIG. 3). The mismatch control oligonucleotide containing 5 bp mismatches (Cur2525) however did not have any anti-tumor effect. This demonstrated in vivo specificity of the LNA-containing antisense oligonucleotide (Cur2524) targeting Ha-ras.

The anti-tumor effect of Cur2524 (LNA-gapmer) was compared with the 16-mer phosphorothioate (Cur2522). Inhibition of tumor growth by Cur2524 (LNA-gapmer) was demonstrated, while the iso-sequential 16-mer phosphorothioate had no effect (FIG. 3).

### Example 21

### In Vivo Superiority of Short LNA Oligomeric Compounds Compared to Longer Phosphorthioate Compound

ISIS 2503 is a well-known antisense oligonucleotide developed by ISIS pharmaceuticals that inhibits expression of Ha-Ras and that compound selected for clinical trials. This oligonucleotide has shown to inhibit tumour growth in several tumour xenograft models e.g. the 15PC3 xenografts (Fluiter et al. Cancer Res. 62, 2024-2028). The goal of this study was to compare the established ISIS 2503 with a LNA gapmer oligomeric compound that targets Ha-Ras in a nude mice model. A further goal was to investigate the potency of short (16-mer) LNA oligomeric compounds compared to a long phosphorothioate (20-mer).

Experimental Design

The following oligonucleotides were synthesized. Cur 2119 is identical to ISIS2503. The oligonucleotides were fully thiolated. It is important to note that the LNA gapmers are 16mers while benchmark oligonucleotides are 20 mers. The compounds were checked using MALDI-TOF analysis (data not shown). The compounds were sufficiently purified for use in the in vivo experiments.

TABLE 4
LNA compounds as 16-mers and benchmark phosphorothioate as 20-mer

| | | Internal | |
|---|---|---|---|
| | | number & | |
| | | seq design | |
| Seq ID No | Sequence (5′-3′) | NO | Length and design |
| 77 | tccgtcatcgctcctcaggg | Cur2119 | PS/DNA 20-mer |
| | | 77D | |
| 75 | TCCGtcatcgctCCTC | Cur2131 | β-D-oxy-LNA (captured letters)/DNA |
| | | 75A | gapmer 16-mer full thiolated |

In Vivo Tumor Growth Inhibition

Eight to ten week old NMRI nu/nu mice (Charles River, the Netherlands) were injected subcutaneously in the flank with 10⁶ MiaPaca II cells or 10⁶ 15PC3 cells in 300 μl Matrigel (Collaborative Biomedical products, Bedford, Mass., USA). The cells were injected within one hour after harvesting by trypsine treatment. Before injection the cells were washed with cold PBS, counted with a haemocytometer and subsequently mixed with the Matrigel on ice. One week after tumor cell injection, when tumor take was positive, an osmotic mini pump (Alzet model 1002, lot. number10017-00, Alzet corp., Palo Alto, Calif., USA) was implanted dorsally according to the instructions of the manufacturer. The osmotic minipumps were incubated in PBS 20 hours at 37° C. prior to implantation to start up the pump. The osmotic minipumps were filled with oligonucleotides (1 mg/kg/day) or 0.9% saline. Tumor growth was monitored daily following the implantation of the osmotic mini pump. Tumor volume was measured and calculated as described previously (Meyer, et al. Int J. Cancer, 43: 851-856, 1989.). All mice were implanted with IPTT-200 temperature transponder chips (BMDS inc., Seaford, Del., USA) to allow temperature measurements and identification of the mice using a DAS 5002 scanner (BMDS inc.) during treatment.

Nude mice were injected s.c. with Miapaca II cells (right flank) and 15PC3 cells (left Flank) one week prior to the start of ODN treatment to allow xenograft growth. The anti Ha-Ras compounds (Cur 2119 and Cur 2131) and controls (Cur 2120 and Cur 2132) were administrated for 14 days using Alzet osmotic minipumps (model 1002) implanted dorsally. Dosages used were 1 mg/kg/day. During treatment the tumor growth was monitored.

It can be concluded that the 16mer LNA containing gapmer is more potent as the 20-mer phosphorthioate oligonucleotide (see FIG. 4).

### Example 22

### In Vivo Potency of Alpha-LNA Oligomeric Compounds are at Least as Good as the Beta-D-Oxy LNA Oligomeric Compounds

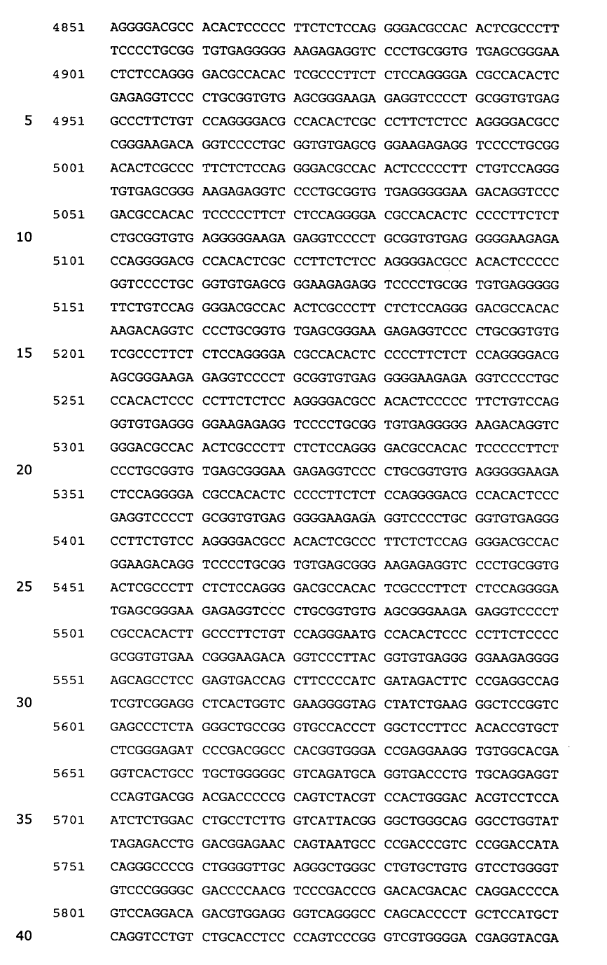

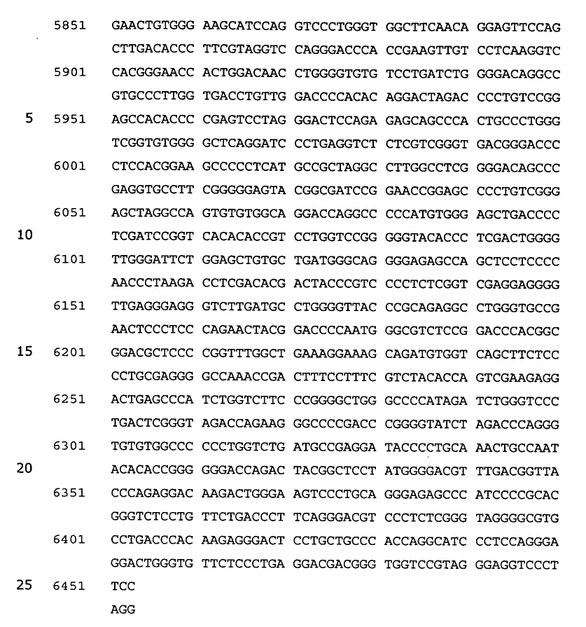

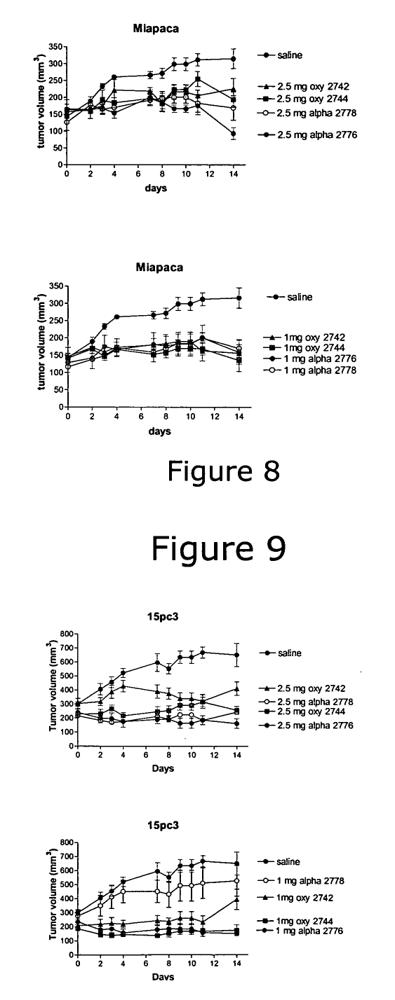

Nude mice were injected s.c. with MiaPaca II cells (right flank) and 15PC3 cells (left flank) one week prior to the start of oligonucleotide treatment to allow xenograft growth. The anti HaRas oligonucleotides (2713, 2722, 2742 and 2776) and control oligonucleotides (2744 and 2778) (see table 5) were administrated for 14 days using Alzet osmotic minipumps (model 1002) implanted dorsally. Three dosages were used: 1, 2.5 and 5 mg/Kg/day for all of them, except for 2722 and 2713, for which a dosage of 5 mg/Kg/day was administered. During treatment the tumor growth was monitored. Tumor growth was almost inhibited completely at 5 mg/Kg/day, 2.5 mg/Kg/day and even at 1 mg/Kg/day dose with 2742 and 2776 in 15PC3 cells, FIG. 8. The specificity with control oligonucleotides (2744 and 2778, containing mismatches) increased as the dose decreased. At 1 mg/Kg/day dose the experiment presented a good specificity, particularly for alpha-L-oxy-LNA oligonucleotides (2742 and 2744). In MiaPacaII xenograft tumors, the effect of the oligonucleotides is in general comparable with those on the 15PC3 xenografts, except for the fact that the specificity seemed to be a bit lower. For 2713 and 2722, a potent inhibition of tumor growth was also observed, see FIG. 9. It can be concluded that the oligonucleotide containing alpha-L-oxy-LNA are as potent, or maybe even better, as the one containing beta-D-oxy-LNA in tumor growth inhibition in the concentration range tested.

TABLE 5

Oligonucleotides containing alpha-L-oxy-LNA(capital letters and ^{a})
and beta-D-oxy-LNA (capital letters) used in the in vivo experiment.
Residue c is methyl-c both for DNA and LNA, except for c DNA in 2713
and 2722.

| | | |
|---|---|---|
| | Internal ref | |
| | & SeqID + | |
| Seq ID NO | design NO | oligonucleotides |

| | | | |
|---|---|---|---|
| 75 | 2776 | T^{a}ₛC^{a}ₛC^{a}ₛgₛtₛcₛaₛtₛcₛgₛcₛtₛC^{a}ₛC^{a}ₛT^{a}ₛc | match |
| | 75F | | |
| 77 | 2778 | T^{a}ₛC^{a}ₛT^{a}ₛgₛtₛaₛaₛtₛaₛgₛcₛcₛC^{a}ₛC^{a}ₛC^{a}ₛc | Mismatch control |
| | 77F | | |
| 75 | 2742 | TₛCₛCₛgₛtₛcₛaₛtₛcₛgₛcₛtₛCₛCₛTₛc | match |
| | 75B | | |
| 77 | 2744 | TₛCₛTₛgₛtₛaₛaₛtₛaₛgₛcₛcₛCₛCₛCₛc | Mismatch control |
| | 77B | | |
| 6 | 2713 | CₛCₛCₛAₛtₛcₛtₛgₛtₛgₛcₛcₛCₛGₛAₛC | Match |
| | 6A | | |
| 15 | 2722 | TₛCₛCₛTₛcₛcₛtₛtₛcₛcₛgₛtₛtₛCₛTₛGₛC | Match |
| | 15A | | |

### Example 23

### Alpha-L-Oxy-LNA and Beta-D-Oxy-LNA Targeting Ha-Ras Show Low Toxicity Levels in Mice

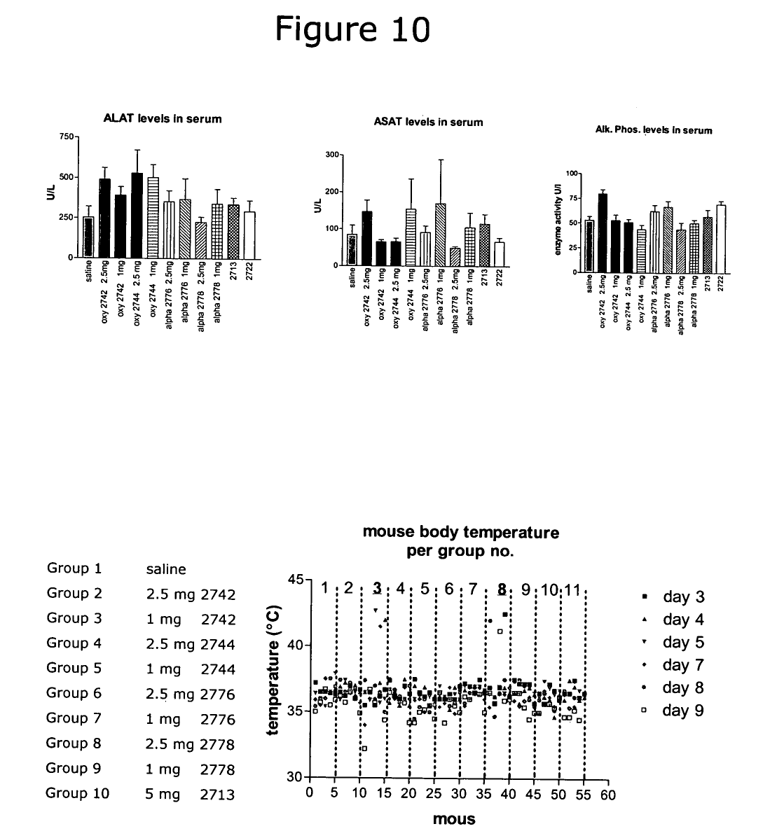

The levels of aspartate aminotransferase (ASAT), alanine aminotransferase (ALAT) and alkaline phosphatase in the serum were determined, in order to study the possible effects of this 14-day treatment in the nude mice. Serum samples were taken from each mouse after the 14-day experiment. ALAT levels in the serum varied between 250-500 U/L. ASAT levels were in the range of 80-150 U/L (see FIG. 10). The mice did not seem externally to be sick, and no big changes in behavior were observed. During treatment the body temperature of the mice was also monitored using IPTT-200 temperature transponders (FIG. 10). The body temperature did not change significantly during the treatment, not even at high dose 5 mg/Kg/day, which is an indication that no major toxicity effects are occurring.

The present invention has been described with specificity in accordance with certain of its preferred embodiments. Therefore, the following examples serve only to illustrate the invention and are not intended to limit the same.

1. A compound consisting of a total of 8-50 nucleotides and/or nucleotide analogues, wherein said compound comprises a subsequence of at least 8 nucleotides or nucleotide analogues, said subsequence being located within a sequence selected from the group consisting of SEQ ID NOS: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75.

2. A compound of claim 1, which modulates the expression of ras selected from Ha-ras, Ki-ras or N-ras.

3. A compound consisting of a total of 8-50 nucleotides and/or nucleotide analogues targeted to a nucleic acid molecule encoding Ha-ras, wherein said compound specifically hybridises with a nucleic acid encoding Ha-ras and inhibits the expression of Ha-ras and wherein said compound comprises a subsequence of at least 8 nucleotides or nucleotide analogues, said subsequence being located within a sequence selected from the group consisting of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75.

4. The compound according to claim 1, which is an antisense oligonucleotide.

5. The compound according to claim 1, comprising at least nucleotide analogue.

6. The compound according to claim 1, comprising at least one Locked Nucleic Acid (LNA) unit.

7. The compound according to claims 6, wherein the Locked Nucleic Acid (LNA) has the structure of the general formula 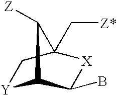
X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected form hydrogen and C₁₋₄-alkyl;
Z and Z* are independently absent, selected among an internucleoside linkage, a terminal group or a protecting group;
B constitutes a natural or non-natural nucleobase;
and the asymmetric groups may be found in either orientation.

8. The compound according to claim 6, wherein at least one nucleotide comprises a Locked Nucleic Acid (LNA) unit according any of the formulas 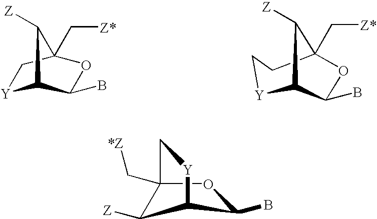
wherein Y is —O—, —S—, —NH—, or N(R^{H}); Z and Z* are independently absent, selected among an internucleoside linkage, a terminal group or a protecting group; and B constitutes a natural or non-natural nucleobase.

9. The compound according to claim 6 or 7, wherein the internucleoside linkage may be selected from the group consisting of —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected form hydrogen and C₁₋₄-alkyl.

10. The compound according to claim 5, 6 or 7, wherein the nucleobases is a modified nucleobases selected from the group consisting of 5-methylcytosine, isocytosine, pseudoisocytosine, 5-bromouracil, 5-propynyluracil, 6-aminopurine, 2-aminopurine, inosine, diaminopurine, 2-chloro-6-aminopurine.

11. The compound according to any of claims 6-8, wherein the LNA is oxy-LNA, thio-LNA, amino-LNA, in either the D-β or L-α configurations or combinations thereof.

12. A compound consisting of a total of 8-50 nucleotides and/or nucleotide analogues, wherein said compound comprises a subsequence of at least 8 nucleotides or nucleotide analogues, said subsequence being located within a sequence selected from the group consisting of SEQ ID NO: 2, 3, 4, 5, 6, 7, 8, 9, 10, 11, 12, 13, 14, 15 and 75.

13. The compound according to claim 1, wherein the antisense oligonucleotide is a design according to any of the designs presented in FIG. 1.

14. The compound according to claim 12, wherein the antisense oligonucleotide is a gapmer.

15. The compound according to claim 1, wherein the antisense oligonucleotide comprises 13, 14, 15, 16, 17, 18, 19, 20 or 21 nucleotides.

16. The compound according to claim 1, comprising 2, 3, 4, 5, 6, 7, 8, 9, 10, 11, 12, 13, 14, 15, 16, 17, 18, 19, 20 or 21 LNA units.

17. The compound according to claim 1, wherein the subsequence is SEQ ID NO: 93.

18. The compound according to claim 1, wherein the subsequence is SEQ ID NO: 96.

19. The compound according to claim 1, wherein the subsequence is SEQ ID NO: 99.

20. The compound according to claim 1, wherein the subsequence is SEQ ID NO: 102.

21. The compound according to claim 1, wherein the subsequence is SEQ ID NO: 105.

22. The compound according to claim 1, wherein the subsequence is SEQ ID NO: 108.

23. The compound according to claim 1, wherein the subsequence is SEQ ID NO: 111.

24. The compound according to claim 1, wherein the subsequence is SEQ ID NO: 114.

25. The compound according to claim 1, wherein the subsequence is SEQ ID NO: 117.

26. The compound according to claim 1, wherein the subsequence is SEQ ID NO: 120.

27. The compound according to claim 1, wherein the subsequence is SEQ ID NO: 123.

28. The compound according to claim 1, wherein the subsequence is SEQ ID NO: 126.

29. The compound according to claim 1, wherein the subsequence is SEQ ID NO: 129.

30. The compound according to claim 1, wherein the subsequence is SEQ ID NO: 132.

31. The compound according to claim 1, wherein the 3′ end LNA is replaced by the corresponding natural nucleoside.

32. A compound consisting of SEQ ID NO: 93.

33. A compound consisting of SEQ ID NO: 96.

34. A compound consisting of SEQ ID NO: 99.

35. A compound consisting of SEQ ID NO: 102.

36. A compound consisting of SEQ ID NO: 105.

37. A compound consisting of SEQ ID NO: 108.

38. A compound consisting of SEQ ID NO: 111.

39. A compound consisting of SEQ ID NO: 114.

40. A compound consisting of SEQ ID NO: 117.

41. A compound consisting of SEQ ID NO: 120.

42. A compound consisting of SEQ ID NO: 123.

43. A compound consisting of SEQ ID NO: 126.

44. A compound consisting of SEQ ID NO: 129.

45. A compound consisting of SEQ ID NO: 132.

50. The compound according to any of claims 34-45, wherein the 3′ end LNA is replaced by the corresponding nucleotide.

51. A conjugate comprising the compound according to claim 1 and at least one non-nucleotide or non-polynucleotide moiety covalently attached to said compound.

52. A pharmaceutical composition comprising a compound as defined in claim 1 or a conjugate as defined in claim 59, and a pharmaceutically acceptable diluent, carrier or adjuvant.

53. The pharmaceutical composition according to claim 51 further comprising at least one chemotherapeutic agent.

54. The pharmaceutical composition according to claim 52, wherein said chemotherapeutic compound is selected from the group consisting of adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine).

55. A pharmaceutical composition comprising the compound of claim 1, which further comprises a pharmaceutically acceptable carrier.

56. A pharmaceutical composition comprising the compound of claim 1, which is employed in a pharmaceutically acceptable salt.

57. A pharmaceutical composition comprising the compound of claim 1, which is constitutes a pro-drug.

58. A pharmaceutical composition comprising the compound of claim 1, which further comprises an antiinflamatory compounds and/or antiviral compounds.

59. Use of a compound as defined in claim 1 or as conjugate as defined in claim 51 for the manufacture of a medicament for the treatment of cancer.

60. Use according to claim 59, wherein said cancer is in the form of a solid tumor.

61. Use according to claim 59 or 60, wherein said cancer is a carcinoma.

62. Use according to claim 61, wherein said carcinoma is selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors.

63. Use according to claim 62 wherein said carcinoma is selected from the group consisting of malignant melanoma, non-small cell lung cancer, breast carcinoma, colon carcinoma and renal cell carcinoma.

64. Use according to claim 63, wherein said malignant melanoma is selected from the group consisting of superficial spreading melanoma, nodular melanoma, lentigo maligna melanoma, acral melagnoma, amelanotic melanoma and desmoplastic melanoma.

65. Use according to claim 60 or 61, wherein said cancer is a sarcoma.

66. Use according to claim 65, wherein said sarcoma is selected from the group consisting of osteosarcoma, Ewing's sarcoma, chondrosarcoma, malignant fibrous histiocytoma, fibrosarcoma and Kaposi's sarcoma.

67. Use according to claim 60 or 61, wherein said cancer is a glioma.

68. A method for treating cancer, said method comprising administering a compound as defined in claim 1, a conjugate as defined in claim 51 or a pharmaceutical composition as defined in any of claims 52-58 to a patient in need thereof.

69. The method according to claim 68, wherein said cancer is in the form of a solid tumor.

70. The method according to claim 68 or 69, wherein said cancer is a carcinoma.

71. The method according to claim 70, wherein said carcinoma is selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors.

72. The method according to claim 71, wherein said carcinoma is selected from the group consisting of malignant melanoma, non-small cell lung cancer, breast carcinoma, colon carcinoma and renal cell carcinoma.

73. The method according to claim 72, wherein said malignant melanoma is selected from the group consisting of superficial spreading melanoma, nodular melanoma, lentigo maligna melanoma, acral melagnoma, amelanotic melanoma and desmoplastic melanoma.

74. The method according to claim 68, wherein said cancer is a sarcoma.

75. The method according to claim 74, wherein said sarcoma is selected from the group consisting of osteosarcoma, Ewing's sarcoma, chondrosarcoma, malignant fibrous histiocytoma, fibrosarcoma, artherosclerosis, psoriasis, diabetic retinopathy, rheumatoid arthritis, asthma, warts, allergic dermatitis and Kaposi's sarcoma.

75. The method according to claim 68, wherein said cancer is a glioma.

76. A method of inhibiting the expression of Ha-ras, in cells or tissues comprising contacting said cells or tissues with the compound according to claim 1 so that expression of Ha-ras is inhibited.

77. A method of modulating expression of a gene involved in a cancer disease comprising contacting the gene or RNA from the gene with an oligomeric compound wherein said compound has a sequence comprising at least an 8 nucleobase portion of SEQ ID NO: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75 whereby gene expression is modulated.

78. A method according to claim 77, wherein the compounds comprises one or more LNA units.

79. The method of claim 77 or 78, wherein the compound hybridizes with messenger RNA of the gene to inhibit expression thereof.

80. A method of treating a mammal suffering from or susceptible from an cancer disease, comprising:
administering to the mammal an therapeutically effective amount of an oligonucleotide targeted to Ha-ras that comprises one or more LNA units.

81. The method according to any of the claims 77-80, wherein the cancer diseases is a lung, breast, colon, prostate, pancreas, lung, liver, thyroid, kidney, brain, testes, stomach, intestine, bowel, spinal cord, sinuses, bladder, urinary tract or ovaries cancer.

82. A method of modulating the red blood cell proliferation, cellular proliferation, ion metabolism, glucose and energy metabolism, pH regulation or matrix metabolism comprising contacting a cell with the antisense compound of claim 1 so that the cell is modulated.

83. A method of inhibiting the proliferation of cells comprising contacting cells in vitro with an effective amount of the antisense compound of claim 1, so that proliferation of the cells is inhibited.

84. The method of claim 83 wherein said cells are cancer cells.

85. A method of inhibiting the expression of Ha-ras in human cells or tissues comprising contacting human cells or tissues with the compound of claim 1 so that expression of Ha-ras is inhibited.

86. A method of treating an animal having a disease or condition associated with Ha-ras comprising administering to an animal having a disease or condition associated with Ha-ras a therapeutically or prophylactically effective amount of the antisense compound of claim 1 so that expression of Ha-ras is inhibited.

87. The method of claim 86 wherein the disease or condition is a hyperproliferative condition.

88. The method of claim 87 wherein the hyperproliferative condition is cancer.

89. A method of treating a human having a disease or condition characterized by a reduction in apoptosis comprising administering to a human having a disease or condition characterized by a reduction in apoptosis a prophylactically or therapeutically effective amount of the antisense compound of claim 11.

90. A method of modulating apoptosis in a cell comprising contacting a cell with the antisense compound of claim 1 so that apoptosis is modulated.

91. A method of modulating cytokinesis in a cell comprising contacting a cell with the antisense compound of claim 1 so that cytokinesis is modulated.

92. A method of modulating the cell cycle in a cell comprising contacting a cell with the antisense compound of claim 1 so that the cell cycle is modulated.

93. A method of inhibiting the proliferation of cells comprising contacting cells with an effective amount of the antisense compound of claim 1, so that proliferation of the cells is inhibited.


SEQUENCE LISTING (201 sequences)

SEQ ID NO: 1 - DNA, 6453 bp, Homo sapiens
ggatcccagcctttccccagcccgtagccccgggacctccgcggtgggcggcgccgcgctgccggcgcagggagggcctctggtgcaccggcaccgctgagtcgggttctctcgccggcctgttcccgggagagcccggggccctgctcggagatgccgccccgggcccccagacaccggctccctggccttcctcgagcaaccccgagctcggctccggtctccagccaagcccaaccccgagaggccgcggccctactggctccgcctcccgcgttgctcccggaagccccgcccgaccgcggctcctgacagacgggccgctcagccaaccggggtggggcggggcccgatggcgcgcagccaatggtaggccgcgcctggcagacggacgggcgcggggcggggcgtgcgcaggcccgcccgagtctccgccgcccgtgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtgagtgcgggcagggatcggccgggccgcgcgccctcctcgcccccaggcggcagcaatacgcgcggcgcgggccgggggcgcggggccggcgggcgtaagcggcggcggcggcggcgggtgggtggggccgggcggggcccgcgggcacaggtgagcgggcgtcgggggctgcggcgggcgggggccccttcctccctggggcctgcgggaatccgggccccacccgtggcctcgcgctgggcacggtccccacgccggcgtacccgggagcctcgggcccggcgccctcacacccgggggcgtctgggaggaggcggccgcggccacggcacgcccgggcacccccgattcagcatcacaggtcgcggaccaggccgggggcctcagccccagtgccttttccctctccgggtctcccgcgccgcttctcggccccttcctgtcgctcagtccctgcttcccaggagctcctctgtcttctccagctttctgtggctgaaagatgcccccggttccccgccgggggtgcggggcgctgcccgggtctgccctcccctcggcggcgcctagtacgcagtaggcgctcagcaaatacttgtcggaggcaccagcgccgcggggcctgcaggctggcactagcctgcccgggcacgccgtggcgcgctccgccgtggccagacctgttctggaggacggtaacctcagccctcgggcgcctccctttagcctttctgccgacccagcagcttctaatttgggtgcgtggttgagagcgctcagctgtcagccctgcctttgagggctgggtcccttttcccatcactgggtcattaagagcaagtgggggcgaggcgacagccctcccgcacgctgggttgcagctgcacaggtaggcacgctgcagtccttgctgcctggcgttggggcccagggaccgctgtgggtttgcccttcagatggccctgccagcagctgccctgtggggcctggggctgggcctgggcctggctgagcagggccctccttggcaggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggtgagcctagcgccgccgtccaggtgccagcagctgctgcgggcgagcccaggacacagccaggatagggctggctgcagcccctggtcccctgcatggtgctgtggccctgtctcctgcttcctctagaggaggggagtccctcgtctcagcaccccaggagaggagggggcatgaggggcatgagaggtaccagggagaggctggctgtgtgaactccccccacggaaggtcctgagggggtccctgagccctgtcctcctgcaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacaggtgaaccccgtgaggctggcccgggagcccacgccgcacaggtggggccaggccggctgcgtccaggcaggggcctcctgtcctctctgcgcatgtcctggatgccgctgcgcctgcagcccccgtagccagctctcgctttccacctctcagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcaggtgaggcagctctccaccccacagctagccagggacccgccccgccccgccccagccagggagcagcactcactgaccctctcccttgacacagggcagccgctctggctctagctccagctccgggaccctctgggaccccccgggacccatgtgacccagcggcccctcgcactgtaggtctcccgggacggcagggcagtgagggaggcgagggccggggtctgggctcacgccctgcagtcctgggccgacacagctccggggaaggcggaggtccttggggagagctgccctgagccaggccggagcggtgaccctggggcccggcccctcttgtccccagagtgtcccacgggcacctgttggttctgagtcttagtggggctactggggacacgggccgtagctgagtcgagagctgggtgcagggtggtcaaaccctggccagacctggagttcaggagggccccgggccaccctgacctttgaggggctgctgtagcatgatgcgggtggccctgggcacttcgagatggccagagtccagcttcccgtgtgtgtggtgggcctggggaagtggctggtggagtcgggagcttcgggccaggcaaggcttgatcccacagcagggagcccctcacccaggcaggcggccacaggccggtccctcctgatcccatccctcctttcccagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttgatcttggttttcggctgagggtgggacacggtgcgcgtgtggcctggcatgaggtatgtcggaacctcaggcctgtccagccctgggctctccatagcctttgggagggggaggttgggagaggccggtcaggggtctgggctgtggtgctctctcctcccgcctgccccagtgtccacggcttctggcagagagctctggacaagcaggcagatcataaggacagagagcttactgtgcttctaccaactaggagggcgtcctggtcctccagagggaggtggtttcaggggttggggatctgtgccggtggctctggtctctgctgggagccttcttggcggtgagaggcatcacctttcctgacttgctcccagcgtgaaatgcacctgccaagaatggcagacatagggaccccgcctcctgggccttcacatgcccagttttcttcggctctgtggcctgaagcggtctgtggaccttggaagtagggctccagcaccgactggcctcaggcctctgcctcattggtggtcgggtagcggccagtagggcgtgggagcctggccatccctgcctcctggagtggacgaggttggcagctggtccgtctgctcctgccccactctcccccgcccctgccctcaccctacccttgccccacgcctgcctcatggctggttgctcttggagcctggtagtgtcactggctcagccttgctgggtatacacaggctctgccacccactctgctccaaggggcttgccctgccttgggccaagttctaggtctggccacagccacagacagctcagtcccctgtgtggtcatcctggcttctgctgggggcccacagcgcccctggtgcccctcccctcccagggcccgggttgaggctgggccaggccctctgggacggggacttgtgccctgtcagggttccctatccctgaggttgggggagagctagcagggcatgccgctggctggccagggctgcagggacactcccccttttgtccagggaataccacactcgcccttctctccagcgaacaccacactcgcccttctctccaggggacgccacactcccccttctgtccaggggacgccacactcccccttctctccaggggacgccacactcgcccttctctccaggggacgccacactcgcccttctctccaggggacgccacactcgcccttctgtccaggggacgccacactcgcccttctctccaggggacgccacactcgcccttctctccaggggacgccacactcccccttctgtccaggggacgccacactcccccttctctccaggggacgccacactcccccttctctccaggggacgccacactcgcccttctctccaggggacgccacactcccccttctgtccaggggacgccacactcgcccttctctccaggggacgccacactcgcccttctctccaggggacgccacactcccccttctctccaggggacgccacactcccccttctctccaggggacgccacactcccccttctgtccaggggacgccacactcgcccttctctccaggggacgccacactcccccttctctccaggggacgccacactcccccttctctccaggggacgccacactcccccttctgtccaggggacgccacactcgcccttctctccaggggacgccacactcgcccttctctccaggggacgccacactcgcccttctctccaggggacgccacacttgcccttctgtccagggaatgccacactcccccttctccccagcagcctccgagtgaccagcttccccatcgatagacttcccgaggccaggagccctctagggctgccgggtgccaccctggctccttccacaccgtgctggtcactgcctgctgggggcgtcagatgcaggtgaccctgtgcaggaggtatctctggacctgcctcttggtcattacggggctgggcagggcctggtatcagggccccgctggggttgcagggctgggcctgtgctgtggtcctggggtgtccaggacagacgtggaggggtcagggcccagcacccctgctccatgctgaactgtgggaagcatccaggtccctgggtggcttcaacaggagttccagcacgggaaccactggacaacctggggtgtgtcctgatctggggacaggccagccacaccccgagtcctagggactccagagagcagcccactgccctgggctccacggaagccccctcatgccgctaggccttggcctcggggacagcccagctaggccagtgtgtggcaggaccaggcccccatgtgggagctgaccccttgggattctggagctgtgctgatgggcaggggagagccagctcctccccttgagggagggtcttgatgcctggggttacccgcagaggcctgggtgccgggacgctccccggtttggctgaaaggaaagcagatgtggtcagcttctccactgagcccatctggtcttcccggggctgggccccatagatctgggtccctgtgtggcccccctggtctgatgccgaggatacccctgcaaactgccaatcccagaggacaagactgggaagtccctgcagggagagcccatccccgcaccctgacccacaagagggactcctgctgcccaccaggcatccctccagggatcc

SEQ ID NO: 2 - DNA, 16 bp, Artificial Sequence
  Synthetic oligonucleotide
attcgtccacaaaatg

SEQ ID NO: 3 - DNA, 16 bp, Artificial Sequence
  Synthetic oligonucleotide
caaaatggttctggat

SEQ ID NO: 4 - DNA, 16 bp, Artificial Sequence
  Synthetic oligonucleotide
cgtattcgtccacaaa

SEQ ID NO: 5 - DNA, 16 bp, Artificial Sequence
  Synthetic oligonucleotide
cacacacaggaagccc

SEQ ID NO: 6 - DNA, 16 bp, Artificial Sequence
  Synthetic oligonucleotide
cccatctgtgcccgac

SEQ ID NO: 7 - DNA, 16 bp, Artificial Sequence
  Synthetic oligonucleotide
tgatggcaaacacaca

SEQ ID NO: 8 - DNA, 16 bp, Artificial Sequence
  Synthetic oligonucleotide
agacttggtgttgttg

SEQ ID NO: 9 - DNA, 16 bp, Artificial Sequence
  Synthetic oligonucleotide
gtccttcacccgtttg

SEQ ID NO: 10 - DNA, 16 bp, Artificial Sequence
  Synthetic oligonucleotide
cgtcatccgagtcctt

SEQ ID NO: 11 - DNA, 16 bp, Artificial Sequence
  Synthetic oligonucleotide
agccaggtcacacttg

SEQ ID NO: 12 - DNA, 16 bp, Artificial Sequence
  Synthetic oligonucleotide
gccgagattccacagt

SEQ ID NO: 13 - DNA, 16 bp, Artificial Sequence
  Synthetic oligonucleotide
catcctccactccctg

SEQ ID NO: 14 - DNA, 16 bp, Artificial Sequence
  Synthetic oligonucleotide
atctcacgcaccaacg

SEQ ID NO: 15 - DNA, 16 bp, Artificial Sequence
  Synthetic oligonucleotide
tcctccttccgtctgc

SEQ ID NO: 16 - DNA, 16 bp, Artificial Sequence
  Synthetic oligonucleotide
ggtctcctgccccacc

SEQ ID NO: 17 - DNA, 16 bp, Artificial Sequence
  Synthetic oligonucleotide
cggggtcctcctacag

SEQ ID NO: 18 - DNA, 16 bp, Artificial Sequence
  Synthetic oligonucleotide
tcaggggcctgcggcc

SEQ ID NO: 19 - DNA, 16 bp, Artificial Sequence
  Synthetic oligonucleotide
attccgtcatcgctcc

SEQ ID NO: 20 - DNA, 16 bp, Artificial Sequence
  Synthetic oligonucleotide
accaccaccagcttat

SEQ ID NO: 21 - DNA, 16 bp, Artificial Sequence
  Synthetic oligonucleotide
cacaccgccggcgccc

SEQ ID NO: 22 - DNA, 16 bp, Artificial Sequence
  Synthetic oligonucleotide
tcagcgcactcttgcc

SEQ ID NO: 23 - DNA, 16 bp, Artificial Sequence
  Synthetic oligonucleotide
gtccacaaaatggttc

SEQ ID NO: 24 - DNA, 16 bp, Artificial Sequence
  Synthetic oligonucleotide
tagtggggtcgtattc

SEQ ID NO: 25 - DNA, 16 bp, Artificial Sequence
  Synthetic oligonucleotide
cggtaggaatcctcta

SEQ ID NO: 26 - DNA, 16 bp, Artificial Sequence
  Synthetic oligonucleotide
aatgaccacctgcttc

SEQ ID NO: 27 - DNA, 16 bp, Artificial Sequence
  Synthetic oligonucleotide
ggcacgtctccccatc

SEQ ID NO: 28 - DNA, 16 bp, Artificial Sequence
  Synthetic oligonucleotide
tccaggatgtccaaca

SEQ ID NO: 29 - DNA, 16 bp, Artificial Sequence
  Synthetic oligonucleotide
ctcctggccggcggta

SEQ ID NO: 30 - DNA, 16 bp, Artificial Sequence
  Synthetic oligonucleotide
gcatggcgctgtactc

SEQ ID NO: 31 - DNA, 16 bp, Artificial Sequence
  Synthetic oligonucleotide
cgcatgtactggtccc

SEQ ID NO: 32 - DNA, 16 bp, Artificial Sequence
  Synthetic oligonucleotide
gaagccctccccggtg

SEQ ID NO: 33 - DNA, 16 bp, Artificial Sequence
  Synthetic oligonucleotide
tggcaaacacacacag

SEQ ID NO: 34 - DNA, 16 bp, Artificial Sequence
  Synthetic oligonucleotide
gacttggtgttgttga

SEQ ID NO: 35 - DNA, 16 bp, Artificial Sequence
  Synthetic oligonucleotide
gtggatgtcctcaaaa

SEQ ID NO: 36 - DNA, 16 bp, Artificial Sequence
  Synthetic oligonucleotide
tctgctccctgtactg

SEQ ID NO: 37 - DNA, 16 bp, Artificial Sequence
  Synthetic oligonucleotide
tccttcacccgtttga

SEQ ID NO: 38 - DNA, 16 bp, Artificial Sequence
  Synthetic oligonucleotide
gggcacgtcatccgag

SEQ ID NO: 39 - DNA, 16 bp, Artificial Sequence
  Synthetic oligonucleotide
tccccaccagcaccat

SEQ ID NO: 40 - DNA, 16 bp, Artificial Sequence
  Synthetic oligonucleotide
gccaggtcacacttgt

SEQ ID NO: 41 - DNA, 16 bp, Artificial Sequence
  Synthetic oligonucleotide
ttccacagtgcgtgca

SEQ ID NO: 42 - DNA, 16 bp, Artificial Sequence
  Synthetic oligonucleotide
cctgagcctgccgaga

SEQ ID NO: 43 - DNA, 16 bp, Artificial Sequence
  Synthetic oligonucleotide
tagcttcgggcgaggt

SEQ ID NO: 44 - DNA, 16 bp, Artificial Sequence
  Synthetic oligonucleotide
gatgtaggggatgccg

SEQ ID NO: 45 - DNA, 16 bp, Artificial Sequence
  Synthetic oligonucleotide
tcttggccgaggtctc

SEQ ID NO: 46 - DNA, 16 bp, Artificial Sequence
  Synthetic oligonucleotide
tccactccctgccggg

SEQ ID NO: 47 - DNA, 16 bp, Artificial Sequence
  Synthetic oligonucleotide
cgtgtagaaggcatcc

SEQ ID NO: 48 - DNA, 16 bp, Artificial Sequence
  Synthetic oligonucleotide
ggatctcacgcaccaa

SEQ ID NO: 49 - DNA, 16 bp, Artificial Sequence
  Synthetic oligonucleotide
cgcagcttgtgctgcc

SEQ ID NO: 50 - DNA, 16 bp, Artificial Sequence
  Synthetic oligonucleotide
aggagggttcagcttc

SEQ ID NO: 51 - DNA, 16 bp, Artificial Sequence
  Synthetic oligonucleotide
cggggccactctcatc

SEQ ID NO: 52 - DNA, 16 bp, Artificial Sequence
  Synthetic oligonucleotide
ttgcagctcatgcagc

SEQ ID NO: 53 - DNA, 16 bp, Artificial Sequence
  Synthetic oligonucleotide
tcaggagagcacacac

SEQ ID NO: 54 - DNA, 16 bp, Artificial Sequence
  Synthetic oligonucleotide
ctgagcttgtgctgcg

SEQ ID NO: 55 - DNA, 16 bp, Artificial Sequence
  Synthetic oligonucleotide
ccggcacctccatgtc

SEQ ID NO: 56 - DNA, 16 bp, Artificial Sequence
  Synthetic oligonucleotide
cacctccttcctgcat

SEQ ID NO: 57 - DNA, 16 bp, Artificial Sequence
  Synthetic oligonucleotide
ctcctccttccgtctg

SEQ ID NO: 58 - DNA, 16 bp, Artificial Sequence
  Synthetic oligonucleotide
cttccgtccttccttc

SEQ ID NO: 59 - DNA, 16 bp, Artificial Sequence
  Synthetic oligonucleotide
cttccttccttccttg

SEQ ID NO: 60 - DNA, 16 bp, Artificial Sequence
  Synthetic oligonucleotide
ctgggctccagcagcc

SEQ ID NO: 61 - DNA, 16 bp, Artificial Sequence
  Synthetic oligonucleotide
cacggtcccggggtga

SEQ ID NO: 62 - DNA, 16 bp, Artificial Sequence
  Synthetic oligonucleotide
tgcagtcacctcggcc

SEQ ID NO: 63 - DNA, 16 bp, Artificial Sequence
  Synthetic oligonucleotide
cctccctgggagggtc

SEQ ID NO: 64 - DNA, 16 bp, Artificial Sequence
  Synthetic oligonucleotide
gacagtctgtgcacag

SEQ ID NO: 65 - DNA, 16 bp, Artificial Sequence
  Synthetic oligonucleotide
catttgggatgttcaa

SEQ ID NO: 66 - DNA, 16 bp, Artificial Sequence
  Synthetic oligonucleotide
gctggggttccggtgg

SEQ ID NO: 67 - DNA, 16 bp, Artificial Sequence
  Synthetic oligonucleotide
gggaggggagctaagg

SEQ ID NO: 68 - DNA, 16 bp, Artificial Sequence
  Synthetic oligonucleotide
gggcccacagaggcct

SEQ ID NO: 69 - DNA, 16 bp, Artificial Sequence
  Synthetic oligonucleotide
catctgtgcccgacaa

SEQ ID NO: 70 - DNA, 16 bp, Artificial Sequence
  Synthetic oligonucleotide
taatttactgtgatcc

SEQ ID NO: 71 - DNA, 16 bp, Artificial Sequence
  Synthetic oligonucleotide
tttcaagaccatccaa

SEQ ID NO: 72 - DNA, 16 bp, Artificial Sequence
  Synthetic oligonucleotide
tggatcagctggatgg

SEQ ID NO: 73 - DNA, 16 bp, Artificial Sequence
  Synthetic oligonucleotide
cacaccgtcggcgccc

SEQ ID NO: 74 - DNA, 16 bp, Artificial Sequence
  Synthetic oligonucleotide
ctccaggccggcggta

SEQ ID NO: 75 - DNA, 16 bp, Artificial Sequence
  Synthetic oligonucleotide
tccgtcatcgctcctc

SEQ ID NO: 76 - DNA, 16 bp, Artificial Sequence
  Synthetic oligonucleotide
tcagtaatagccccac

SEQ ID NO: 77 - DNA, 20 bp, Artificial Sequence
  Synthetic oligonucleotide
tccgtcatcgctcctcaggg

SEQ ID NO: 78 - DNA, 18 bp, Artificial Sequence
  Synthetic oligonucleotide primer
gccggatgcaggaaggag

SEQ ID NO: 79 - DNA, 19 bp, Artificial Sequence
  Synthetic oligonucleotide primer
gctccagcagcccttcctt

SEQ ID NO: 80 - DNA, 28 bp, Artificial Sequence
  Synthetic oligonucleotide probe
cgtccttccttcctcctccttccgtctg

SEQ ID NO: 81 - DNA, 21 bp, Artificial Sequence
  Synthetic oligonucleotide primer
aaggctgtgggcaaggtcatc

SEQ ID NO: 82 - DNA, 23 bp, Artificial Sequence
  Synthetic oligonucleotide primer
gtcagatccacgacggacacatt

SEQ ID NO: 83 - DNA, 34 bp, Artificial Sequence
  Synthetic oligonucleotide probe
gaagctcactggcatggcatggccttccgtgttc

SEQ ID NO: 84 - DNA, 20 bp, Artificial Sequence
  Synthetic oligonucleotide primer
aatctcggcaggctcaggac

SEQ ID NO: 85 - DNA, 23 bp, Artificial Sequence
  Synthetic oligonucleotide primer
gggatgttcaagacagtctgtgc

SEQ ID NO: 86 - DNA, 18 bp, Artificial Sequence
  Synthetic oligonucleotide primer
aacggatttggtcgtatt

SEQ ID NO: 87 - DNA, 18 bp, Artificial Sequence
  Synthetic oligonucleotide primer
taagcagttggtggtgca

SEQ ID NO: 88 - DNA, 20 bp, Artificial Sequence
  Synthetic oligonucleotide
tccgtcatcgctcctcaggg

SEQ ID NO: 89 - DNA, 20 bp, Artificial Sequence
  Synthetic oligonucleotide
tcagtaatagccccacatgg

SEQ ID NO: 90 - DNA, 16 bp, Artificial Sequence
  Synthetic oligonucleotide
tccgtcatcgctcctc

SEQ ID NO: 91 - DNA, 16 bp, Artificial Sequence
  Synthetic oligonucleotide
tccgtcatcgctcctc

SEQ ID NO: 92 - DNA, 16 bp, Artificial Sequence
  Synthetic oligonucleotide
tcagtaatagccccac

SEQ ID NO: 93 - DNA, 16 bp, Artificial Sequence
  Synthetic oligonucleotide
attcgtccacaaaatg

SEQ ID NO: 94 - DNA, 16 bp, Artificial Sequence
  Synthetic oligonucleotide
attcgtccacaaaatg

SEQ ID NO: 95 - DNA, 16 bp, Artificial Sequence
  Synthetic oligonucleotide
attcgtccacaaaatg

SEQ ID NO: 96 - DNA, 16 bp, Artificial Sequence
  Synthetic oligonucleotide
caaaatggttctggat

SEQ ID NO: 97 - DNA, 16 bp, Artificial Sequence
  Synthetic oligonucleotide
caaaatggttctggat

SEQ ID NO: 98 - DNA, 16 bp, Artificial Sequence
  Synthetic oligonucleotide
caaaatggttctggat

SEQ ID NO: 99 - DNA, 16 bp, Artificial Sequence
  Synthetic oligonucleotide
cgtattcgtccacaaa

SEQ ID NO: 100 - DNA, 16 bp, Artificial Sequence
  Synthetic oligonucleotide
cgtattcgtccacaaa

SEQ ID NO: 101 - DNA, 16 bp, Artificial Sequence
  Synthetic oligonucleotide
cgtattcgtccacaaa

SEQ ID NO: 102 - DNA, 16 bp, Artificial Sequence
  Synthetic oligonucleotide
cacacacaggaagccc

SEQ ID NO: 103 - DNA, 16 bp, Artificial Sequence
  Synthetic oligonucleotide
cacacacaggaagccc

SEQ ID NO: 104 - DNA, 16 bp, Artificial Sequence
  Synthetic oligonucleotide
cacacacaggaagccc

SEQ ID NO: 105 - DNA, 16 bp, Artificial Sequence
  Synthetic oligonucleotide
cccatctgtgcccgac

SEQ ID NO: 106 - DNA, 16 bp, Artificial Sequence
  Synthetic oligonucleotide
cccatctgtgcccgac

SEQ ID NO: 107 - DNA, 16 bp, Artificial Sequence
  Synthetic oligonucleotide
cccatctgtgcccgac

SEQ ID NO: 108 - DNA, 16 bp, Artificial Sequence
  Synthetic oligonucleotide
tgatggcaaacacaca

SEQ ID NO: 109 - DNA, 16 bp, Artificial Sequence
  Synthetic oligonucleotide
tgatggcaaacacaca

SEQ ID NO: 110 - DNA, 16 bp, Artificial Sequence
  Synthetic oligonucleotide
tgatggcaaacacaca

SEQ ID NO: 111 - DNA, 16 bp, Artificial Sequence
  Synthetic oligonucleotide
agacttggtgttgttg

SEQ ID NO: 112 - DNA, 16 bp, Artificial Sequence
  Synthetic oligonucleotide
agacttggtgttgttg

SEQ ID NO: 113 - DNA, 16 bp, Artificial Sequence
  Synthetic oligonucleotide
agacttggtgttgttg

SEQ ID NO: 114 - DNA, 16 bp, Artificial Sequence
  Synthetic oligonucleotide
gtccttcacccgtttg

SEQ ID NO: 115 - DNA, 16 bp, Artificial Sequence
  Synthetic oligonucleotide
gtccttcacccgtttg

SEQ ID NO: 116 - DNA, 16 bp, Artificial Sequence
  Synthetic oligonucleotide
gtccttcacccgtttg

SEQ ID NO: 117 - DNA, 16 bp, Artificial Sequence
  Synthetic oligonucleotide
cgtcatccgagtcctt

SEQ ID NO: 118 - DNA, 16 bp, Artificial Sequence
  Synthetic oligonucleotide
cgtcatccgagtcctt

SEQ ID NO: 119 - DNA, 16 bp, Artificial Sequence
  Synthetic oligonucleotide
cgtcatccgagtcctt

SEQ ID NO: 120 - DNA, 16 bp, Artificial Sequence
  Synthetic oligonucleotide
agccaggtcacacttg

SEQ ID NO: 121 - DNA, 16 bp, Artificial Sequence
  Synthetic oligonucleotide
agccaggtcacacttg

SEQ ID NO: 122 - DNA, 16 bp, Artificial Sequence
  Synthetic oligonucleotide
agccaggtcacacttg

SEQ ID NO: 123 - DNA, 16 bp, Artificial Sequence
  Synthetic oligonucleotide
gccgagattccacagt

SEQ ID NO: 124 - DNA, 16 bp, Artificial Sequence
  Synthetic oligonucleotide
gccgagattccacagt

SEQ ID NO: 125 - DNA, 16 bp, Artificial Sequence
  Synthetic oligonucleotide
gccgagattccacagt

SEQ ID NO: 126 - DNA, 16 bp, Artificial Sequence
  Synthetic oligonucleotide
catcctccactccctg

SEQ ID NO: 127 - DNA, 16 bp, Artificial Sequence
  Synthetic oligonucleotide
catcctccactccctg

SEQ ID NO: 128 - DNA, 16 bp, Artificial Sequence
  Synthetic oligonucleotide
catcctccactccctg

SEQ ID NO: 129 - DNA, 16 bp, Artificial Sequence
  Synthetic oligonucleotide
atctcacgcaccaacg

SEQ ID NO: 130 - DNA, 16 bp, Artificial Sequence
  Synthetic oligonucleotide
atctcacgcaccaacg

SEQ ID NO: 131 - DNA, 16 bp, Artificial Sequence
  Synthetic oligonucleotide
atctcacgcaccaacg

SEQ ID NO: 132 - DNA, 16 bp, Artificial Sequence
  Synthetic oligonucleotide
tcctccttccgtctgc

SEQ ID NO: 133 - DNA, 16 bp, Artificial Sequence
  Synthetic oligonucleotide
tcctccttccgtctgc

SEQ ID NO: 134 - DNA, 16 bp, Artificial Sequence
  Synthetic oligonucleotide
tcctccttccgtctgc

SEQ ID NO: 135 - DNA, 16 bp, Artificial Sequence
  Synthetic oligonucleotide
tccgtcatcgctcctc

SEQ ID NO: 136 - DNA, 16 bp, Artificial Sequence
  Synthetic oligonucleotide
tctgtaatagcccccc

SEQ ID NO: 137 - DNA, 16 bp, Artificial Sequence
  Synthetic oligonucleotide
tccgtcatcgctcctc

SEQ ID NO: 138 - DNA, 16 bp, Artificial Sequence
  Synthetic oligonucleotide
tccgtcatcgctcctc

SEQ ID NO: 139 - DNA, 16 bp, Artificial Sequence
  Synthetic oligonucleotide
tctgtaatagcccccc

SEQ ID NO: 140 - DNA, 16 bp, Artificial Sequence
  Synthetic oligonucleotide
tccgtcatcgctcctc

SEQ ID NO: 141 - DNA, 18 bp, Artificial sequence
  poly-T oligonucleotide
tttttttttttttttttt

SEQ ID NO: 142 - DNA, 16 bp, Artificial Sequence
  Synthetic oligonucleotide
ggtctcctgccccacc

SEQ ID NO: 143 - DNA, 16 bp, Artificial Sequence
  Synthetic oligonucleotide
cggggtcctcctacag

SEQ ID NO: 144 - DNA, 16 bp, Artificial Sequence
  Synthetic oligonucleotide
tcaggggcctgcggcc

SEQ ID NO: 145 - DNA, 16 bp, Artificial Sequence
  Synthetic oligonucleotide
attccgtcatcgctcc

SEQ ID NO: 146 - DNA, 16 bp, Artificial Sequence
  Synthetic oligonucleotide
accaccaccagcttat

SEQ ID NO: 147 - DNA, 16 bp, Artificial Sequence
  Synthetic oligonucleotide
cacaccgccggcgccc

SEQ ID NO: 148 - DNA, 16 bp, Artificial Sequence
  Synthetic oligonucleotide
tcagcgcactcttgcc

SEQ ID NO: 149 - DNA, 16 bp, Artificial Sequence
  Synthetic oligonucleotide
gtccacaaaatggttc

SEQ ID NO: 150 - DNA, 16 bp, Artificial Sequence
  Synthetic oligonucleotide
tagtggggtcgtattc

SEQ ID NO: 151 - DNA, 16 bp, Artificial Sequence
  Synthetic oligonucleotide
cggtaggaatcctcta

SEQ ID NO: 152 - DNA, 16 bp, Artificial Sequence
  Synthetic oligonucleotide
aatgaccacctgcttc

SEQ ID NO: 153 - DNA, 16 bp, Artificial Sequence
  Synthetic oligonucleotide
ggcacgtctccccatc

SEQ ID NO: 154 - DNA, 16 bp, Artificial Sequence
  Synthetic oligonucleotide
tccaggatgtccaaca

SEQ ID NO: 155 - DNA, 16 bp, Artificial Sequence
  Synthetic oligonucleotide
ctcctggccggcggta

SEQ ID NO: 156 - DNA, 16 bp, Artificial Sequence
  Synthetic oligonucleotide
gcatggcgctgtactc

SEQ ID NO: 157 - DNA, 16 bp, Artificial Sequence
  Synthetic oligonucleotide
cgcatgtactggtccc

SEQ ID NO: 158 - DNA, 16 bp, Artificial Sequence
  Synthetic oligonucleotide
gaagccctccccggtg

SEQ ID NO: 159 - DNA, 16 bp, Artificial Sequence
  Synthetic oligonucleotide
tggcaaacacacacag

SEQ ID NO: 160 - DNA, 16 bp, Artificial Sequence
  Synthetic oligonucleotide
gacttggtgttgttga

SEQ ID NO: 161 - DNA, 16 bp, Artificial Sequence
  Synthetic oligonucleotide
gtggatgtcctcaaaa

SEQ ID NO: 162 - DNA, 16 bp, Artificial Sequence
  Synthetic oligonucleotide
tctgctccctgtactg

SEQ ID NO: 163 - DNA, 16 bp, Artificial Sequence
  Synthetic oligonucleotide
tccttcacccgtttga

SEQ ID NO: 164 - DNA, 16 bp, Artificial Sequence
  Synthetic oligonucleotide
gggcacgtcatccgag

SEQ ID NO: 165 - DNA, 16 bp, Artificial Sequence
  Synthetic oligonucleotide
tccccaccagcaccat

SEQ ID NO: 166 - DNA, 16 bp, Artificial Sequence
  Synthetic oligonucleotide
gccaggtcacacttgt

SEQ ID NO: 167 - DNA, 16 bp, Artificial Sequence
  Synthetic oligonucleotide
ttccacagtgcgtgca

SEQ ID NO: 168 - DNA, 16 bp, Artificial Sequence
  Synthetic oligonucleotide
cctgagcctgccgaga

SEQ ID NO: 169 - DNA, 16 bp, Artificial Sequence
  Synthetic oligonucleotide
tagcttcgggcgaggt

SEQ ID NO: 170 - DNA, 16 bp, Artificial Sequence
  Synthetic oligonucleotide
gatgtaggggatgccg

SEQ ID NO: 171 - DNA, 16 bp, Artificial Sequence
  Synthetic oligonucleotide
tcttggccgaggtctc

SEQ ID NO: 172 - DNA, 16 bp, Artificial Sequence
  Synthetic oligonucleotide
tccactccctgccggg

SEQ ID NO: 173 - DNA, 16 bp, Artificial Sequence
  Synthetic oligonucleotide
cgtgtagaaggcatcc

SEQ ID NO: 174 - DNA, 16 bp, Artificial Sequence
  Synthetic oligonucleotide
ggatctcacgcaccaa

SEQ ID NO: 175 - DNA, 16 bp, Artificial Sequence
  Synthetic oligonucleotide
cgcagcttgtgctgcc

SEQ ID NO: 176 - DNA, 16 bp, Artificial Sequence
  Synthetic oligonucleotide
aggagggttcagcttc

SEQ ID NO: 177 - DNA, 16 bp, Artificial Sequence
  Synthetic oligonucleotide
cggggccactctcatc

SEQ ID NO: 178 - DNA, 16 bp, Artificial Sequence
  Synthetic oligonucleotide
ttgcagctcatgcagc

SEQ ID NO: 179 - DNA, 16 bp, Artificial Sequence
  Synthetic oligonucleotide
tcaggagagcacacac

SEQ ID NO: 180 - DNA, 16 bp, Artificial Sequence
  Synthetic oligonucleotide
ctgagcttgtgctgcg

SEQ ID NO: 181 - DNA, 16 bp, Artificial Sequence
  Synthetic oligonucleotide
ccggcacctccatgtc

SEQ ID NO: 182 - DNA, 16 bp, Artificial Sequence
  Synthetic oligonucleotide
cacctccttcctgcat

SEQ ID NO: 183 - DNA, 16 bp, Artificial Sequence
  Synthetic oligonucleotide
ctcctccttccgtctg

SEQ ID NO: 184 - DNA, 16 bp, Artificial Sequence
  Synthetic oligonucleotide
cttccgtccttccttc

SEQ ID NO: 185 - DNA, 16 bp, Artificial Sequence
  Synthetic oligonucleotide
cttccttccttccttg

SEQ ID NO: 186 - DNA, 16 bp, Artificial Sequence
  Synthetic oligonucleotide
ctgggctccagcagcc

SEQ ID NO: 187 - DNA, 16 bp, Artificial Sequence
  Synthetic oligonucleotide
cacggtcccggggtga

SEQ ID NO: 188 - DNA, 16 bp, Artificial Sequence
  Synthetic oligonucleotide
tgcagtcacctcggcc

SEQ ID NO: 189 - DNA, 16 bp, Artificial Sequence
  Synthetic oligonucleotide
cctccctgggagggtc

SEQ ID NO: 190 - DNA, 16 bp, Artificial Sequence
  Synthetic oligonucleotide
gacagtctgtgcacag

SEQ ID NO: 191 - DNA, 16 bp, Artificial Sequence
  Synthetic oligonucleotide
catttgggatgttcaa

SEQ ID NO: 192 - DNA, 16 bp, Artificial Sequence
  Synthetic oligonucleotide
gctggggttccggtgg

SEQ ID NO: 193 - DNA, 16 bp, Artificial Sequence
  Synthetic oligonucleotide
gggaggggagctaagg

SEQ ID NO: 194 - DNA, 16 bp, Artificial Sequence
  Synthetic oligonucleotide
gggcccacagaggcct

SEQ ID NO: 195 - DNA, 16 bp, Artificial Sequence
  Synthetic oligonucleotide
catctgtgcccgacaa

SEQ ID NO: 196 - DNA, 16 bp, Artificial Sequence
  Synthetic oligonucleotide
taatttactgtgatcc

SEQ ID NO: 197 - DNA, 16 bp, Artificial Sequence
  Synthetic oligonucleotide
tttcaagaccatccaa

SEQ ID NO: 198 - DNA, 16 bp, Artificial Sequence
  Synthetic oligonucleotide
tggatcagctggatgg

SEQ ID NO: 199 - DNA, 16 bp, Artificial Sequence
  Synthetic oligonucleotide
cacaccgtcggcgccc

SEQ ID NO: 200 - DNA, 16 bp, Artificial Sequence
  Synthetic oligonucleotide
ctccaggccggcggta

SEQ ID NO: 201 - DNA, 16 bp, Artificial Sequence
  Synthetic oligonucleotide
tccgtcatcgctcctc